(19) United States
(12) Patent Application Publication
(10) Pub. No.: US 2006/0068409 A1
(43) Pub. Date: Mar. 30, 2006

(54) Use of restriction enzymes and other chemical methods to decrease non-specific binding in dual bead assays and related bio-discs, methods, and system apparatus for detecting medical targets

(76) Inventors: Brigitte Chau Phan, Irvine, CA (US); Jorma Antero Virtanen, Las Vegas, NV (US); Amethyst Hoang Lam, Irvine, CA (US); KaYuen Yeung, San Francisco, CA (US); James Howard Coombs, Irvine, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 11/069,760

(22) Filed: Feb. 28, 2005

Related U.S. Application Data
(63) Continuation of application No. 10/099,266, filed on Mar. 14, 2002, now abandoned.
Application No. 10/099,266 is a continuation-in-part of application No. 09/997,741, filed on Nov. 27, 2001.
(60) Provisional application No. 60/272,525, filed on Mar. 1, 2001.
(60) Provisional application No. 60/253,958, filed on Nov. 28, 2000.
(60) Provisional application No. 60/253,283, filed on Nov. 27, 2000.

(51) Int. Cl. C12Q 1/68; C12M 1/34

(52) U.S. Cl. 435/6; 435/287.2

(57) ABSTRACT

Methods for decreasing non-specific bindings of beads in dual bead assays and related optical bio-discs and disc drive systems. Methods include identifying whether a target agent is present in a biological sample and mixing capture beads each having at least one transport probe affixed thereto, reporter beads each having at least one signal probe affixed thereto, and a biological sample. Mixing is performed under binding conditions to permit formation of a dual bead complex if the target agent is present in the sample. The reporter bead and capture bead each are bound to the target agent. Denaturing the target agent and keeping it in the denatured form by use of a specialized hybridization buffer is also provided. A denaturing agent is guanidine isothiocynate. Methods further include isolating the dual bead complex from the mixture to obtain an isolate, exposing the isolate to a capture field on a disc, and detecting the presence of the dual bead complex in the disc to indicate that the target agent is present in the sample. The methods may further include selectively breaking up non-specific binding between capture beads and reporter beads employing a digestion agent. Also employed is a method for selectively breaking up non-specific binding between capture beads and reporter beads using a wash buffer containing a chemical agent. The methods are applied to detecting medical targets.

### CROSS-REFERENCE TO RELATED APPLICATIONS

This application is a continuation of U.S. patent application Ser. No. 10/099,266, entitled USE OF RESTRICTION ENZYMES AND OTHER CHEMICAL METHODS TO DECREASE NON—SPECIFIC BINDING IN DUAL BEAD ASSAYS AND RELATED BIO-DISCS, METHODS, AND SYSTEM APPARATUS FOR DETECTING MEDICAL TARGETS, filed Mar. 14, 2002, which is a continuation-in-part of U.S. patent application Ser. No. 09/997,741, entitled DUAL BEAD ASSAYS INCLUDING OPTICAL BIODISCS AND METHODS RELATING THERETO, filed Nov. 27, 2001, which is a non-provisional application which claims priority U.S. Provisional Patent Application No. 60/272,525, filed Mar. 1, 2001, U.S. Provisional Patent Application No. 60/253,958, filed Nov. 28, 2000, and U.S. Provisional Patent Application No. 60/253,283, filed Nov. 27, 2000.

This application also claims priority to U.S. Provisional Patent Application No. 60/352,270, filed Jan. 30, 2002, U.S. Provisional Application Ser. No. 60/314,906 filed Aug. 24, 2001, U.S. Provisional Patent Application No. 60/278,697, filed Mar. 26, 2001, U.S. Provisional Patent Application No. 60/278,110, filed Mar. 23, 2001, U.S. Provisional Patent Application No. 60/278,106, filed Mar. 23, 2001 and U.S. Provisional Patent Application No. 60/275,643, filed Mar. 14, 2001. The foregoing non-provisional and provisional patent applications to which this application claims priority are incorporated herein by reference in their entireties.

### BACKGROUND OF THE INVENTION

1. Field of the Invention

The present invention relates to optical analysis discs, optical bio-discs, medical CDs, and related methods. The invention further relates to methods for decreasing non-specific binding in dual bead assays by use of restriction enzymes and other chemical methods. The current method also relates to the use of denaturing agents to increase sensitivity of a dual bead assay. The present methods are performed by employing optical bio-discs and related system apparatus. Certain aspects of the present invention are directed to the detection of medical targets. The present methods utilizing magnetic or metal beads may be implemented on a magneto-optical bio-disc.

2. Discussion of the Related Art

There is a significant need to make diagnostic assays and forensic assays of all types faster and more local to the end-user. Ideally, clinicians, patients, investigators, the military, other health care personnel, and consumers should be able to test themselves for the presence of certain factors or indicators in their systems, and for the presence of certain biological material at a crime scene or on a battlefield. At present, there are a number of silicon-based chips with nucleic acids and/or proteins attached thereto, which are commercially available or under development. These chips are not for use by the end-user, or for use by persons or entities lacking very specialized expertise and expensive equipment.

### SUMMARY OF THE INVENTION

The present invention relates to performing assays, and particularly to using dual bead or multi-bead structures on a disc. The invention includes methods for preparing assays, methods for performing assays, discs for performing assays, and related detection systems.

In one aspect, the present invention includes methods for determining whether a target agent is present in a biological sample. These methods can include mixing capture beads each having at least one transport probe, reporter beads each having at least one signal probe, and a biological sample. These components are mixed under binding conditions that permit formation of a dual bead complex if the target agent is present in the sample. The dual bead complex thus includes a reporter bead and a capture bead each bound to the target agent. The dual bead complex is isolated from the mixture to obtain an isolate. The isolate is then exposed to a capture field on an optical disc. The capture field has a capture agent that binds specifically to the signal probe or transport probe of the dual bead complex. The dual bead complex in the optical disc is then detected to indicate that the target agent is present in the sample and, if desired, to indicate a concentration.

The capture beads can have a specified size and have a characteristic that makes them “isolatable”. The capture beads are preferably magnetic, in which case the isolating of dual bead complex (and some capture beads not part of a complex) in a mixture includes subjecting the mixture to a magnetic field with a permanent magnet, an electromagnet, or a magnetic array of capture points written on a magneto-optical bio-disc according to certain aspects of the present invention.

The reporter bead should have characteristics that make it identifiable and distinguishable with detection. The reporter beads can be made of one of a number of materials, such as latex, gold, plastic, steel, or titanium, and should have a known and specified size. The reporter beads can be fluorescent and can be yellow, green, red, or blue, for example.

The dual bead complex can be formed on the disc itself, or outside the disc and added to the disc. To form the dual bead complex off disc, methods referred to here as “single-step” or “two-step” can be employed. In the two-step method, the mixture initially includes capture beads and the sample. The capture beads are then isolated to wash away unbound sample and leave bound and unbound capture beads in a first isolate. Reporter beads are then added to the first isolate to produce dual bead complex structures and the isolation process is repeated. The resulting isolate leaves dual bead complex with reporters, but also includes unbound capture beads without reporters. The reporters make the dual bead complex detectable.

In the “single-step” method, the capture beads, reporter beads, and sample are mixed together from the start and then the isolation process isolates dual bead complex along with unbound capture beads.

These methods for producing and isolating dual bead complex structures can be performed on the disc. The sample and beads can be added to the disc together, or the beads can be pre-loaded on the disc so that only a sample needs to be added. The sample and beads can be added in a mixing chamber on the disc, and the disc can be rotated in one direction or in both to assist the mixing. An isolate can then be created, such as by applying an electromagnet and rotating to cause the material other than the capture beads to be moved to a waste chamber. The isolate is then directed through rotation to capture fields.

The dual bead complex structures can be detected on the capture field by use of various methods. In one embodiment, the detecting includes directing a beam of electromagnetic energy from a disc drive toward the capture field and analyzing electromagnetic energy returned from or transmitted past the reporter bead of the dual bead complex attached to the capture field. The disc drive assembly can include a detector and circuitry or software that senses the detector signal for a sufficient transition between light and dark (referred to as an “event”) to spot a reporter bead.

Beads can, alternatively, be detected based on their fluorescence. In this case, the energy source in the disc drive preferably has a wavelength controllable light source and a detector that is or can be made specific to a particular wavelength. Alternatively, a disc drive can be made with a specific light source and detector to produce a dedicated device, in which case the source may only need fine-tuning.

The biological sample can include blood, serum, plasma, cerebrospinal fluid, breast aspirate, synovial fluid, pleural fluid, perintoneal fluid, pericardial fluid, urine, saliva, amniotic fluid, semen, mucus, a hair, feces, a biological particulate suspension, a single-stranded or double-stranded nucleic acid molecule, a cell, an organ, a tissue or a tissue extract, or any other sample that includes a target that may be bound through chemical or biological processes. Further details relating to other aspects associated with the selection and detection of various targets is disclosed in, for example, commonly assigned co-pending U.S. Provisional Patent Application Ser. No. 60/278,697 entitled “Dual Bead Assays for Detecting Medical Targets” filed Mar. 26, 2001, which is incorporated herein by reference in its entirety.

In addition to these medical uses, the embodiments of the present invention can be used in other ways, such as for testing for impurities in a sample, such as food or water, or for otherwise detecting the presence of a material, such as a biological warfare agent.

The target agent can include, for example, a nucleic acid such as DNA or RNA, or a protein such as an antigen or an antibody. If the target agent is a nucleic acid, both the transport probe and the signal probe may be a nucleic acid molecule complementary to the target nucleic acid. If the target agent is a protein, both the transport probe and the signal probe can be an antibody that specifically binds the target protein.

The transport probe or signal probe can bind specifically to the capture agent on the optical disc due to a high affinity between the probe and the capture agent. This high affinity can, for example, be the result of a strong protein-protein affinity (i.e., antigen-antibody affinity), or the result of a complementarity between two nucleic acid molecules.

Preferably the binding is to the signal probe, and then the disc is rotated to move unbound structures, including capture beads not bound to reporter beads, away from the capture field. If the binding is to the transport probe, unbound capture beads may be included, although the reporter beads are still the beads that are detected. This may be acceptable if the detection is for producing a qualitative or yes/no answer, or if a fine concentration detection is not otherwise required.

The transport probe and signal probe can each be one or more probes selected from the group consisting of single-stranded DNA, double-stranded DNA, single-stranded RNA, peptide nucleic acid, biotin, streptavidin, an antigen, an antibody, a receptor protein, and a ligand. In a further embodiment, each transport probe includes double-stranded DNA and single-stranded DNA, wherein the double-stranded DNA is proximate to the capture layer of the optical disc and the single-stranded DNA is distal relative to the capture layer of the optical disc.

The reporter bead and/or signal probe can be biotinylated and the capture agent can include streptavidin or Neutravidin. Chemistry for affixing capture agents to the capture layer of the optical disc are generally known, especially in the case of affixing a protein or nucleic acid to solid surfaces. The capture agent can be affixed to the capture layer by use of an amino group or a thiol group.

The target agent can include a nucleic acid characteristic of a disease, or a nucleotide sequence specific for a person, or a nucleotide sequence specific for an organism, which may be a bacterium, a virus, a mycoplasm, a fungus, a plant, or an animal. The target agent can include a nucleic acid molecule associated with cancer in a human. The target nucleic acid molecule can include a nucleic acid, which is at least a portion of a gene selected from the group consisting of HER2neu, p52, p53, p21, and bcl-2. The target agent can be an antibody that is present only in a subject infected with HIV-1, a viral protein antigen, or a protein characteristic of a disease state in a subject. The methods and apparatus of the present invention can be used for determining whether a subject is infected by a virus, whether nucleic acid obtained from a subject exhibits a single nucleotide mutation (SNM) relative to corresponding wild-type nucleic acid sequence, or whether a subject expresses a protein of interest, such as a bacterial protein, a fungal protein, a viral protein, an HIV protein, a hepatitis C protein, a hepatitis B protein, or a protein known to be specifically associated with a disease. An example of a dual bead experiment detecting a nucleic acid target is presented below in Example 1.

According to another aspect of the invention, there is provided multiplexing methods wherein more than one target agent (e.g., tens, hundreds, or even thousands of different target agents) can be identified on one optical analysis disc. Multiple capture agents can be provided in a single chamber together in capture fields, or separately in separate capture fields. To be distinguishable from each other, different types of reporter beads can be utilized in these multiplexing methods. This includes, for example, beads that fluoresce at different wavelengths or different size reporter beads. Experiments were performed to identify two different targets using the multiplexing technique. An example of one such assay is discussed below in Example 2.

In accordance with yet another aspect of the invention, there is provided an optical disc with a substrate, a capture layer associated with the substrate, and a capture agent bound to the capture layer, such that the capture agent binds to a dual bead complex. Multiple different capture agents can be used for different types of dual bead complexes. The disc can be designed to allow for some dual bead processing on the disc with appropriate chambers and fluidic structures, and can be pre-loaded with reporter and capture beads so that only a sample needs to be added to form the dual bead complex structures.

According to still a further aspect of this invention, there is provided a disc and disc drive system for performing dual bead assays. The disc drive can include an electromagnet for performing the isolation process, and may include appropriate light source control and detection for the type of reporter beads used. The disc drive can be optical or magneto-optical.

For processing performed on the disc, the drive may advantageously include an electromagnet, and the disc preferably has a mixing chamber, a waste chamber, and capture area. In this embodiment, the sample is mixed with beads in the mixing chamber, a magnetic field is applied adjacent the mixing chamber, and the sample not held by the magnet is directed to the waste chamber so that all magnetic beads, whether bound into a dual bead complex or unbound, remain in the mixing chamber. The magnetic beads are then directed to the capture area. One of a number of different valving arrangements can be used to control the flow.

In still another aspect of the present invention, a bio-disc or medical CD (also “Med-CD”) is produced for use with biological samples and is used in conjunction with a disc drive, such as a magneto-optical disc drive, that can form magnetic regions on a disc. In a magneto-optical disc and drive, magnetic regions can be formed in a highly controllable and precise manner. These regions may be employed advantageously to magnetically bind magnetic beads, including unbound magnetic capture beads or including dual bead complexes with magnetic capture beads. The magneto-optical disc drive can write to selected locations on the disc, and then use an optical reader to detect features located at those regions. The regions can be erased, thereby allowing the beads to be released.

In still another aspect, the invention includes a method for use with a bio-disc and drive including forming magnetic regions on the bio-disc, and providing magnetic beads to the discs so that the beads bind at the magnetic locations. The method preferably further includes detecting at the locations where the magnetic beads bind biological samples, preferably using reporter beads that are detectable, such as by fluorescence or optical event detection. The method can be formed in multiple stages in terms of time or in terms of location through the use of multiple chambers. The regions are written to and a sample is moved over the magnetic regions in order to capture magnetic beads. The regions can then be erased and released if desired. This method allows many different tests to be performed at one time, and can allow a level of interactivity between the user and the disc drives such that additional tests can be created during the testing process.

In accordance with yet another principal aspect of this invention, there is provided methods for enhancing the sensitivity of the dual bead assay. There are two major factors that limit the sensitivity of the dual bead assays. The first is non-specific binding of the capture beads to the reporters in the absence of target DNA. The second factor is the low target-mediated binding of reporter beads to capture beads. Numerous approaches were investigated to circumvent these obstacles.

The modifications implemented herein significantly reduce the non-specific binding of reporter beads to the capture beads in the absence of target. These modifications are executed either prior or during the dual bead assay. However, since this prevention is not absolute, we have introduced a method to selectively break up or disassociate non-specific binding after the dual bead assay, but prior to target quantification. The principle for this selective action relies on the hypothesis that the non-specific binding between the reporter and capture beads is mediated by hydrophobic interactions between the two solid phases, whereas the specific binding is mediated by base pairing with the target DNA.

To accurately separate the specific binding from non-specific binding, restriction enzymes recognizing specific DNA sequences are used. The restriction enzymes selectively cut the target-mediated bonds, releasing the reporter beads from the capture beads. The restriction enzymes, however, do not have any effect on the non-specific hydrophobic bonds between the capture beads and reporter beads. The separation of the reporter beads that have been released from the capture beads by restriction enzymes is facilitated by the magnetic nature of the capture beads. The reporter beads can then be quantified using the optical disc reader. Data collected from experiments employing restriction enzymes to decrease non-specific binding between the reporter and capture beads are presented below in FIGS. 38 and 39. An example of a dual bead assay performed using restriction enzymes is discussed in detail below in Example 3.

The selective cleavage by restriction enzymes can be easily adapted on the bio-disc or medical CD. The dual bead assay according to the present invention may be quantified on a closed bio-disc. The dual bead assay may be first carried out outside the disk. To capture the dual bead on the disk for quantification, a capture zone is created as illustrated in FIGS. 25A-25D and 26A-26D.

To separate the specific binding from non-specific binding, chemical methods that specifically denature hydrogen bonds between DNA sequences are used. The chemical treatments selectively denature the target-mediated bonds, releasing the reporter beads from the capture beads. They however do not have any effect on the non-specific hydrophobic bonds between the capture beads and reporter beads. The separation of the reporter beads that have been released from the capture beads by chemical treatment is facilitated by the magnetic nature of the capture beads. The reporter beads can then be quantified using the optical disc reader.

The chemical methods that were investigated included use of urea, bases, and acids. By varying the concentration of urea, target-mediated hydrogen bonds between the capture and reporter beads can be disrupted. Varying the pH could also result in selective bond cleaving, such that only the specifically bound reporter beads would be quantified. FIGS. 41, 42A, and 42B show results from various experiments related to use of chemical denaturing agents and its effect on bead binding in a dual bead assay format. An example of a study carried out using acid, base, and urea to reduce non-specific bead binding is discussed below in Example 4.

The selective cleavage by chemical methods can be easily adapted on the bio-disc. The dual bead assay according to the present invention may be quantified on a closed bio-disc. The dual bead assay may be first carried out off-disc. To capture the dual bead on the disk for quantification, a capture zone is created. The dual bead assay suspension is then loaded into the channels via an inlet port such that the whole channel is filled with the sample. The ports are sealed and the disc is rotated in the disc drive assembly. During spinning, all free magnetic capture beads will be spun off to the bottom of the channel. Only the reporter beads (with or without the attaching magnetic capture beads) are captured by the capture zone. The number of reporter beads can be quantified by the optical disc reader. Methods using optical bio-discs to detect beads in a dual bead assay are described in detail in conjunction with FIGS. 25A-25D and 25A-25D.

Another principal aspect of the invention is to further modify the dual bead assays to detect medical targets. In real samples, the DNA targets are double-stranded and very long. The ability of the dual bead assay, as well as for any other DNA diagnostic assays, to detect sequences of clinical interest within the whole genome relies first on the specificity of the probes for the sequence of interest and second on the use of a detergent to keep the DNA target in the denatured, single-stranded, form for capture. Thus a major modification introduced to the dual bead assay includes the use of a denaturing agent in the hybridization buffer to prevent re-annealing of complementary sequences of the target DNA. This allows hybridization between the target and probes. FIGS. 43 and 44 present data gathered from experiments using guanidine isothiocyanate as a denaturing agent.

In yet another principal aspect, the present invention is also addressed to implementing the methods recited above on to an analysis disc, modified optical disc, a medical CD, or a bio-disc. A bio-disc drive assembly may be employed to rotate the disc, read and process any encoded information stored on the disc, and analyze the DNA samples in the flow channel of the bio-disc. The bio-disc drive is thus provided with a motor for rotating the bio-disc, a controller for controlling the rate of rotation of the disc, a processor for processing return signals form the disc, and an analyzer for analyzing the processed signals. The rotation rate and direction of the motor is controlled to achieve the desired rotation of the disc. The bio-disc drive assembly may also be utilized to write information to the bio-disc either before, during, or after the test material in the flow channel and target zones is interrogated by the read beam of the drive and analyzed by the analyzer. The bio-disc may include encoded information for controlling the rotation rate and direction of the disc, providing processing information specific to the type of DNA or other test to be conducted, and for displaying the results on a monitor associated with the bio-drive.

More specifically now, the present invention is directed to a method for identifying whether a target agent is present in a biological sample. This method includes the specific steps of (1) preparing a plurality of capture beads each having at least one transport probe affixed thereto; (2) preparing a plurality of reporter beads each having at least one signal probe and one anchor agent affixed thereto; and (3) mixing the capture beads, the reporter beads, and the sample under binding conditions to permit formation of a dual bead complex, so that when the target agent is present in the sample, the reporter bead and capture bead each are bound to the target agent. This method includes the further steps of (4) isolating the dual bead complex from the mixture; (5) selectively breaking up dual bead complexes bound by the target agent employing a digestion agent thereby dissociating the reporter beads and capture beads; and (6) isolating the dissociated reporter beads from the mixture to obtain an isolate. The method concludes with (7) exposing the isolate to a target zone on an optical bio-disc, the target zone having a capture agent that binds to the anchor agent on the reporter beads thereby maintaining the reporter beads within the target zone; and (8) detecting the presence of the reporter beads in the disc to indicate that the target agent is present in the sample.

According to one aspect of this method, the digestion agent may be a restriction enzyme such as DNAseI. In one implementation of this method, the selective breaking up of non-specific binding between capture beads and reporter beads is performed prior to target quantification.

According to a second principal aspect of this invention, there is provided another method for identifying whether a target agent is present in a biological sample. This second method includes the steps of (1) preparing a plurality of capture beads, each of the capture bead having at least one transport probe affixed thereto; (2) preparing a plurality of reporter beads, each of the reporter beads having at least one signal probe affixed thereto; and (3) mixing the capture beads, the reporter beads, and the sample under binding conditions to permit formation of a dual bead complex, so that when the target agent is present in the sample, the reporter bead and capture bead each are bound to the target agent. The method continues with the steps of (4) selectively breaking up non-specific binding between capture beads and reporter beads by employing a wash buffer containing a dissociation agent during formation of the dual bead complex; (5) isolating the dual bead complex from the mixture to obtain an isolate; and (6) exposing the isolate to a target zone on an optical bio-disc, the target zone having a capture agent that binds to the dual bead complex. The method concludes with detecting the presence of the dual bead complex in the disc to indicate that the target agent is present in the sample. In this method the dissociation agent may be a chemical agent such as an acid, a base, or urea. The acid contained in the wash buffer may preferably be 0.1M acetic acid (pH4), the base contained in the wash buffer may preferably be 0.1M sodium bicarbonate (pH9), and the urea contained in the wash buffer may be preferably 7M urea. In one particular embodiment of this method, the selective breaking up of non-specific binding between capture beads and reporter beads is performed prior to target quantification.

In accordance with another principal aspect of the methods hereof, there is provided yet another method for identifying whether a target agent is present in a biological sample. This additional method includes the steps of (1) preparing a plurality of capture beads, each of the capture beads having at least one transport probe affixed thereto; (2) preparing a plurality of reporter beads, each of the reporter beads having at least one signal probe affixed thereto; (3) denaturing the target agent and maintaining the target agent in the denatured form by employing a hybridization buffer including a denaturing agent; (4) mixing the capture beads, the reporter beads, and the sample under binding conditions to permit formation of a dual bead complex, so that when the target agent is present in the sample, the reporter bead and capture bead each are bound to the target agent; (5) isolating the dual bead complex from the mixture to obtain an isolate; (6) exposing the isolate to a target zone on an optical bio-disc, the target zone having a capture agent that binds to the dual bead complex; and (7) detecting the presence of the dual bead complex in the disc to indicate that the target agent is present in the sample.

In this method, the target agent may be a medical target agent such as blood, serum, plasma, cerebrospinal fluid, breast aspirate, synovial fluid, pleural fluid, perintoneal fluid, pericardial fluid, urine, saliva, amniotic fluid, semen, mucus, a hair, feces, a biological particulate suspension, a single-stranded or double-stranded nucleic acid molecule, a cell, an organ, a tissue or a tissue extract. The denaturing agent employed for denaturing the target agent may be guanidine isothiocynate, 0M to 2.0M guanidine isothiocynate, and preferably 1.5M guanidine isothiocynate.

According to still another principal aspect of the invention, there is provided a first method of preparing a dual bead assay for use in an optical bio-disc. This method includes the steps of (1) providing a mixture of capture beads that have transport probes bound thereto; (2) providing a mixture of reporter beads that have signal probes bound thereto; (3) suspending the mixture of capture beads in a hybridization solution; (4) adding to the mixture a target agent that hybridizes with the transport probes; (5) adding to the mixture the reporter beads; (6) allowing the signal probes to hybridize with the target agent to thereby form a dual bead complex including at least one capture bead and one reporter bead; (7) separating non-specifically bound capture beads and reporter beads employing a wash buffer containing a dissociation agent during formation of the dual bead complex; (8) separating the dual bead complex from unbound reporter beads; (9) removing from the mixture the unbound reporter beads; and (10) loading the mixture including the dual bead complex into an optical bio-disc for analysis. The step of adding the target agent may be performed advantageously after the step of adding the reporter beads. In this method the target agent may be a segment of genetic material, such as, a single strand of DNA, a single strand of DNA including a portion of double stranded DNA, a single strand of RNA, or a single strand of RNA including a portion of double stranded RNA. The capture beads may be magnetic and then the separating step is performed by use of a magnet field, such as one created by a magnet or an electromagnet. The method may include the further step of removing the hybridization solution from the mixture, and then washing the dual bead complex to purify the mixture by further removing unbound material. This process may continue with the further step of adding a buffer solution to the mixture.

In accordance with yet a further aspect of this invention there is provided a method of testing for the presence of a target-DNA in a DNA sample by use of an optical bio-disc. This testing method includes the principal steps of (1) preparing a DNA sample to be tested for the presence of a target-DNA; (2) preparing a plurality of reporter beads each having attached thereto a plurality of strands of signal-DNA and an anchor agent, the target-DNA and the signal-DNA being complementary; (3) preparing a plurality of capture beads each having attached thereto a plurality of transport-DNA, the target-DNA and transport-DNA being complimentary; (4) mixing the DNA sample, the plurality of reporter beads, and the plurality of capture beads to thereby form a test sample, the transport-DNA and the signal-DNA being non-complimentary; and (5) allowing hybridization between the signal-DNA, any target-DNA, and transport-DNA existing in the DNA sample to thereby form a dual bead complex including at least one capture bead and one reporter bead. This method continues with the steps of (6) separating non-specifically bound capture beads and reporter beads employing a wash buffer containing a dissociation agent during formation of the dual bead complex; (7) removing from the test sample reporter beads that are not associated with the dual bead complex; and (8) depositing the test sample in a flow channel of an optical bio-disc which is in fluid communication with a target zone, the target zone including a plurality of capture agents each including an amino group that attaches to an active layer to immobilize the capture agents within the target zone. The method then concludes with (9) allowing any anchor agent to bind with the capture agents so that reporter beads associated with the dual bead complex are maintained within the target zone; and (10) detecting any dual bead complexes in the target zone to thereby determine whether target-DNA is present in the DNA sample.

According to still yet an additional aspect of this invention, there is provided a method of testing for the presence of a target-DNA in a test sample by use of an optical bio-disc. This additional testing method includes the steps of (1) preparing a test sample to be tested for the presence of a target-DNA; (2) preparing a plurality of reporter beads each having attached thereto a plurality of strands of signal-DNA, the target-DNA and the signal-DNA being complementary; (3) preparing a plurality of capture beads each having attached thereto a plurality of transport-DNA and an anchor agent, the target-DNA and transport-DNA being complimentary; and (4) depositing a plurality of capture beads and reporter beads in a mixing chamber, each of the reporter beads and the capture beads including the signal-DNA and the transport-DNA, respectively, being non-complimentary to each other. The method continues with the next steps of (5) depositing the test sample in the mixing chamber of an optical bio-disc which is linked to a target zone by a connecting flow channel allowing any target-DNA existing in the test sample to bind to the signal-DNA and the transport-DNA on the reporter and the capture bead, respectively, to thereby form a dual bead complex; (6) separating non-specifically bound capture beads and reporter beads employing a buffer containing a dissociation agent during formation of the dual bead complex; and (7) rotating the optical bio-disc to cause the dual bead complex to move from the mixing chamber through the flow channel and into the target zone, the target zone including a plurality of capture agents each including an amino group that attaches to an active layer to immobilize the capture agents within the target zone, the capture agent having affinity for the anchor agent. This particular method concludes with (8) allowing any anchor agent to bind with the capture agent so that capture beads associated with dual bead complex are maintained within the capture zone; (9) removing from the target zone reporter beads that are free of any dual bead complex; and (10) detecting any dual bead complex in the target zone to thereby determine whether target-DNA is present in the test sample.

There is yet still a further method according to the present invention. This method is directed to testing for the presence of a target-RNA in a test sample by use of an optical bio-disc. It includes the steps of (1) preparing a test sample to be tested for the presence of a target-RNA; (2) preparing a plurality of reporter beads each having attached thereto a plurality of strands of signal-DNA, the target-RNA and the signal-DNA being complementary; (3) preparing a plurality of capture beads each having attached thereto a plurality of transport-DNA and an anchor agent, the target-RNA and transport-DNA being complimentary; and (4) depositing a plurality of capture beads and reporter beads in a mixing chamber, each of the reporter beads and capture beads including the signal-DNA and the transport-DNA, respectively, being non-complimentary to each other. The method further includes the steps of (5) depositing the test sample in the mixing chamber of an optical bio-disc which is linked to a target zone by a connecting flow channel allowing any target-RNA existing in the test sample to hybridize with the signal-DNA and the transport-DNA on the reporter and the capture bead, respectively, to thereby form a dual bead complex; (6) separating non-specifically bound capture beads and reporter beads employing a buffer containing a dissociation agent during formation of the dual bead complex; (7) rotating the optical bio-disc to cause the dual bead complex to move from the mixing chamber through the flow channel and into the target zone, the target zone including a plurality of capture agents each including an amino group that attaches to an active layer to immobilize the capture agents within the target zone, the capture agent and the anchor agent having affinity to each other; (8) allowing any anchor agent to bind with the capture agent so that capture beads associated with dual bead complex are maintained within the capture zone; (9) removing from the target zone reporter beads that are free of any dual bead complex; and (10) detecting any dual bead complex in the target zone to thereby determine whether target-RNA is present in the test sample.

In accordance with the immunoassay or immunochemical techniques of this invention, there is provided a method of testing for the presence of a target-antigen in a test sample by use of an optical bio-disc. This immunoassay method includes the steps of (1) preparing a test sample to be tested for the presence of a target-antigen; (2) preparing a plurality of reporter beads each having attached thereto a plurality of signal-antibody, the signal-antibody having an affinity to epitopes on the target-antigen; (3) preparing a plurality of capture beads each having attached thereto a plurality of transport-antibody and an anchor agent, the transport-antibody having affinity to epitopes on the target-antigen; and (4) depositing the capture beads and the reporter beads in a mixing chamber of an optical bio-disc, each of the reporter beads and capture beads including the signal-antibody and the transport-antibody, respectively, having no affinity to each other.

This immunoassay also includes the steps of (5) depositing the test sample in the mixing chamber of the optical bio-disc which is linked to a target zone by a connecting flow channel allowing any target-antigen existing in the test sample to bind to the signal-antibody and the transport-antibody on the reporter and the capture bead, respectively, to thereby form a dual bead complex; (6) separating non-specifically bound capture beads and reporter beads employing a buffer containing a dissociation agent during formation of the dual bead complex; and (7) rotating the optical bio-disc to cause the dual bead complex to move from the mixing chamber through the flow channel and into the target zone, the target zone including a plurality of capture agents each including an amino group that attaches to an active layer to immobilize the capture agents within the target zone.

The present immunoassay method concludes with the steps of (8) allowing any anchor agent to bind with the capture agent so that capture beads associated with dual bead complex are maintained within the capture zone; (9) removing from the target zone reporter beads that are free of any dual bead complex; and (10) detecting any dual bead complex in the target zone to thereby determine whether target-antigen is present in the test sample.

In any of the above methods, the dual bead complex may be detected by directing a beam of electromagnetic energy from a disc drive assembly toward the target zone and analyzing electromagnetic energy returned from the target zones. This return energy may be reflected or transmitted. In any of the above methods including a dissociation agent, such agent may be an acid, a base, or urea.

According to one aspect of the manufacturing methods of the present invention, there is provided a method of making an optical bio-disc to test for the presence of a target agent in a test sample. This manufacturing method includes the steps of (1) providing a substrate having a center and an outer edge; (2) encoding information on an information layer associated with the substrate, the encoded information being readable by a disc drive assembly to control rotation of the disc; (3) forming a target zone in association with the substrate, the target zone disposed at a predetermined location relative to the center of the substrate; and (4) depositing an active layer in the target zone. The method further includes (5) depositing a plurality of capture agents in the target zone, each capture agent including an amino group that covalently attaches to the active layer to immobilize the capture agent within the target zone; (6) forming a flow channel in fluid communication with the target zone; (7) forming a mixing chamber in fluid communication with the flow channel; (8) depositing a plurality of reporter beads in the mixing chamber, each of the reporter beads having attached thereto a plurality of signal probes, each of the signal probes having affinity to the target agent; and (9) depositing a plurality of capture beads in the mixing chamber, each of the capture beads having attached thereto a plurality of transport probes and an anchor agent, each of the transport probes having affinity to the target agent, the transport probes and signal probes having no affinity toward each other, and the capture agents and the anchor agents having specific affinity to each other. This method also includes (10) separating non-specifically bound capture beads and reporter beads employing a buffer containing a dissociation agent; and (1) adding a pre-determined amount of blocking agent to the mixing chamber to prevent non-specific binding of the beads to each other and the walls of the mixing chamber.

The present invention also contemplates an optical bio-disc as used in conjunction with any one of the above methods or as used to perform any of the above methods.

There is also provided an optical bio-disc according to other aspects of this invention. One particular embodiment of this bio-disc includes a substrate having encoded information associated therewith. The encoded information is readable by a disc drive assembly to control rotation of the disc. The bio-disc also includes a target zone associated with the substrate. The target zone is preferably disposed at a predetermined location relative to the substrate and an active layer is associated with the target zone. There is also provided a plurality of capture agents attached to the active layer so that when the substrate is rotated, the capture agents remain attached to the active layer to thereby maintain a number of the capture agents within the target zone so that when a dual bead complex (that has been pre-washed in a buffer containing a dissociation agent) is introduced into the target zone, the capture agent sequesters the dual bead complex therein to thereby allow detection of captured dual bead complex.

In the above optical bio-disc, the capture agent may be a single stranded oligonucleotide sequence, a double stranded oligonucleotide sequence, an antibody, an antigen, biotin, and streptavidin. Any of these capture agents may advantageously include an amino group and in this case, the active layer may preferably be formed from a polystyrene-co-maleic anhydride. In this embodiment the amino group chemically reacts with the maleic anhydride to form a covalent bond thereby maintaining the capture agents within the target zone. In this particular embodiment, the capture agent may bind with an anchor agent to thereby locate the anchor agent within the target zone. This anchor agent may be bound to one of two beads forming the dual bead complex that includes a capture bead and a reporter bead. The anchor agent may be associated with the capture bead or alternatively with the reporter bead.

As with the above methods, the dissociation agent associated with the present optical bio-disc may be a chemical agent such as an acid, base, or urea. The acid contained in the wash buffer can preferably be 0.1M acetic acid (pH4), while the base contained in the wash buffer can preferably be 0.1 M sodium bicarbonate (pH9), and the urea contained in the wash buffer is 7M urea. In some preferred embodiments of the present optical bio-disc, the anchor agent is a single stranded oligonucleotide sequence, a double stranded oligonucleotide sequence, an antibody, an antigen, biotin, or streptavidin.

According to another aspect of the manufacturing techniques of the present invention, there is provided a method of making an optical bio-disc for testing for the presence of a target-DNA in a DNA sample. This manufacturing method includes the steps of (1) providing a substrate having a center and an outer edge; (2) encoding information on an information layer associated with the substrate, the encoded information being readable by a disc drive assembly to control rotation of the disc; (3) forming a target zone in association with the substrate, the target zone disposed at a predetermined location relative to the center of the substrate; (4) applying an active layer in the target zone; (5) depositing within the target zone, a plurality of strands of capture-DNA each including an amino group that covalently attaches to the active layer to immobilize the strands of capture-DNA within the target zone; (6) forming a flow channel in fluid communication with the target zone; (7) forming a mixing chamber in fluid communication with the flow channel; (8) depositing a plurality of reporter beads in the mixing chamber, each of the reporters including a signal-DNA that has an affinity for the target-DNA; (9) depositing a plurality of capture beads in the mixing chamber, each of the capture bead including a transport-DNA that hybridizes with a portion of the target-DNA and is complementary to the capture-DNA, the transport-DNA and signal-DNA being non-complimentary; (10) depositing a pre-determined amount of dissociation agent to reduce non-specific binding between the capture beads and the reporter beads; and (11) designating an input site associated with the mixing chamber, the input site implemented to receive a DNA sample to be tested for the presence of any target-DNA. The present invention also contemplates an optical bio-disc made according to this manufacturing method.

During use of the above manufactured disc to perform a DNA assay, when the DNA sample is deposited in the mixing chamber, hybridization occurs between the signal-DNA, the target-DNA, and the transport-DNA to thereby form a dual bead complex including at least one reporter bead and one capture bead. During further use of this disc, when the disc is rotated, the dual bead complex moves into the target zone and hybridization occurs between the anchor-DNA and the capture-DNA to thereby place the dual bead complex in the target zone.

The various embodiments of the discs, system apparatus, and methods of the present invention can be designed for use by an end-user, inexpensively, without specialized expertise and expensive equipment. The system can be made portable, and thus usable in remote locations where traditional diagnostic equipment may not generally be available. Other related aspects applicable to components of this assay system and signal acquisition methods are disclosed in commonly assigned and co-pending U.S. patent application Ser. No. 10/038,297 entitled “Dual Bead Assays Including Covalent Linkages For Improved Specificity And Related Optical Analysis Discs” filed Jan. 4, 2002; U.S. Provisional Application Ser. No. 60/272,525 entitled “Biological Assays Using Dual Bead Multiplexing Including Optical Bio-Disc and Related Methods” filed Mar. 1, 2001; and U.S. Provisional Application Ser. Nos. 60/275,643, 60/314,906, and 60/352,270 each entitled “Surface Assembly for Immobilizing Capture Agents and Dual Bead Assays Including Optical Bio-Disc and Methods Relating Thereto” respectively filed Mar. 14, 2001, Aug. 24, 2001, and Jan. 30, 2002. All of these applications are herein incorporated by reference in their entirety.

Other features and advantages of the different embodiments and aspects of the present invention will become apparent from the following detailed description, accompanying drawing figures, and related examples.

### BRIEF DESCRIPTION OF THE DRAWING FIGURES

Further objects of the present invention together with additional features contributing thereto and advantages accruing therefrom will be apparent from the following description of preferred embodiments of the present invention which are shown in the accompanying drawing figures with like reference numerals indicating like components throughout, wherein:

FIG. 1 is a perspective view of an optical disc system according to the present invention;

FIG. 2 is a block and pictorial diagram of an optical reading system according to embodiments of the present invention;

FIGS. 3A, 3B, and 3C are respective exploded, top, and perspective views of a reflective disc according to embodiments of the present invention;

FIGS. 4A, 4B, and 4C are respective exploded, top, and perspective views of a transmissive disc according to embodiments of the present invention;

FIG. 5A is a partial longitudinal cross sectional view of the reflective optical bio-disc shown in FIGS. 3A, 3B, and 3C illustrating a wobble groove formed therein;

FIG. 5B is a partial longitudinal cross sectional view of the transmissive optical bio-disc illustrated in FIGS. 4A, 4B, and 4C showing a wobble groove formed therein and a top detector;

FIG. 6A is a partial radial cross-sectional view of the disc illustrated in FIG. 5A;

FIG. 6B is a partial radial cross-sectional view of the disc illustrated in FIG. 5B;

FIGS. 7A, 8A, 9A, and 10A are schematic representations of a capture bead, a reporter bead, and a dual bead complex as utilized in conjunction with genetic assays;

FIGS. 7B, 8B, 9B, and 10B are schematic representations of a capture bead, a reporter bead, and a dual bead complex as employed in conjunction with immunochemical assays;

FIGS. 11A-1 and 11A-2 together comprise a pictorial representation of one embodiment of a method for producing genetic dual bead complex solutions;

FIGS. 11B-1 and 11B-2 taken together form a pictorial representation of one embodiment of a method for producing immunochemical dual bead complex solutions;

FIGS. 12A-1 and 12A-2 together show a pictorial representation of another embodiment of a method for producing genetic dual bead complex solutions;

FIGS. 12B-1 and 12B-2 taken together illustrate a pictorial representation of another embodiment of a method for producing immunochemical dual bead complex solutions;

FIG. 13 is a longitudinal cross sectional view illustrating the disc layers in combination with a mixing or loading chamber and an inlet in a reflective disc;

FIG. 14 is a view similar to FIG. 13 showing the mixing chamber loaded with dual bead complex solution;

FIGS. 15A and 15B are radial cross sectional views of the disc and target zone illustrating one embodiment for binding of reporter beads to capture agents in a genetic assay;

FIGS. 16A and 16B are radial cross sectional views of the disc and target zone showing another embodiment for binding of reporter beads to capture agents in a genetic assay;

FIG. 17 is radial cross sectional view of the disc and target zone illustrating one embodiment for binding of capture beads to capture agents in a genetic assay;

FIG. 18 is radial cross sectional view of the disc and target zone depicting another embodiment for binding of capture beads to capture agents in a genetic assay;

FIGS. 19A, 19B, and 19C are partial cross sectional views illustrating one embodiment of a method according to this invention for binding the reporter bead of a dual bead complex to a capture layer in a genetic assay;

FIGS. 20A, 20B, and 20C are partial cross sectional views showing one embodiment of a method according to the present invention for binding the reporter bead of a dual bead complex to a capture layer in a immunochemical assay;

FIGS. 21A, 21B, and 21C are partial cross sectional views illustrating another embodiment of a method according to this invention for binding the reporter bead of a dual bead complex to a capture layer in a genetic assay;

FIGS. 22A, 22B, and 22C are partial cross sectional views presenting another embodiment of a method according to the invention for binding the reporter bead of a dual bead complex to a capture layer in a immunochemical assay;

FIGS. 23A and 23B are partial cross sectional views depicting one embodiment of a method according to the present invention for binding the capture bead of a dual bead complex to a capture layer in a genetic assay;

FIGS. 24A and 24B are partial cross sectional views showing another embodiment of a method according to this invention for binding the capture bead of a dual bead complex to a capture layer in a genetic assay;

FIGS. 25A-25D illustrate a method according to the present invention for detecting the presence of target DNA or RNA in a genetic sample utilizing an optical bio-disc;

FIGS. 26A-26D illustrate another method according to this invention for detecting the presence of target DNA or RNA in a genetic sample utilizing an optical bio-disc;

FIGS. 27A-27D illustrate a method according to the present invention for detecting the presence of a target antigen in a biological test sample utilizing an optical bio-disc;

FIG. 28A is a graphical representation of an individual 2.1 micron reporter bead and a 3 micron capture bead positioned relative to the tracks of an optical bio-disc according to the present invention;

FIG. 28B is a series of signature traces derived from the beads of FIG. 28A utilizing a detected signal from the optical drive according to the present invention;

FIG. 29A is a graphical representation of a 2.1 micron reporter bead and a 3 micron capture bead linked together in a dual bead complex positioned relative to the tracks of an optical bio-disc according to the present invention;

FIG. 29B is a series of signature traces derived from the dual bead complex of FIG. 29A utilizing a detected signal from the optical drive according to this invention;

FIG. 30A is a bar graph showing results from a dual bead assay according to the present invention;

FIG. 30B is a graph showing a standard curve demonstrating the detection limit for fluorescent beads detected with a flourimeter;

FIG. 30C is a pictorial representation demonstrating the formation of the dual bead complex;

FIG. 31 is a bar graph showing the sensitivity of disc drive detection using a dual bead complex;

FIG. 32 is a schematic representation of combining beads for dual bead assay multiplexing according to embodiments of the present invention;

FIG. 33A is a schematic representation of a fluidic circuit according to the present invention utilized in conjunction with a magnetic field generator to control movement of magnetic beads;

FIGS. 33B-33D are schematics of a first fluidic circuit that implements the valving structure of FIG. 33A according to one embodiment of fluid transport aspects of the present invention;

FIGS. 34A-34C are schematics of a second fluidic circuit that implements the valving structure of FIG. 33A according to another embodiment of the fluid transport aspects of this invention;

FIG. 35 is a perspective view of the magnetic field generator and a disc including one embodiment of a fluidic circuit employed in conjunction with magnetic beads according to this invention;

FIGS. 36A, 36B, and 36C are plan views illustrating a method of separation and detection for dual bead assays using the fluidic circuit shown in FIG. 35;

FIG. 37 is a perspective view of a magneto-optical bio-disc showing magnetic regions, magnetically bound capture beads, and the formation of dual bead complexes according to another aspect of the present invention;

FIG. 38 is a bar graph presentation demonstrating the effect of DNAseI digestion in absence of reporter beads;

FIG. 39 is a bar graph presentation showing the efficiency of dual bead assay by the effect of DNAseI enzymes digestion;

FIG. 40 is a schematic representation of separation of reporter beads from capture beads by enzyme digestion and physical or chemical treatments;

FIG. 41 is a bar graph presentation showing dual bead complexes prior to and after washing in a basic solution;

FIG. 42A is a bar graph presentation illustrating dual bead complexes prior to and after washing in a 7M urea solution;

FIG. 42B is a bar graph presentation representing dual bead complexes prior to and after washing in a 7M urea solution including the detection of dissociated reporter beads after the urea wash;

FIG. 43 is a bar graph presentation demonstrating the use of 1.5M guanidine isothiocyanate as a denaturing agent during dual bead assay; and

FIG. 44 is a bar graph presentation showing the varying concentrations of guanidine isothiocyanate employed as a denaturing agent during dual bead assay.

### DETAILED DESCRIPTION OF THE PREFERRED EMBODIMENTS

The following description of the present invention relates to optical analysis discs, disc drive systems, and assay chemistries and techniques. The invention further relates to alternate magneto-optical drive systems, MO bio-discs, and related processing methods.

Disc Drive System and Related Optical Analysis Discs

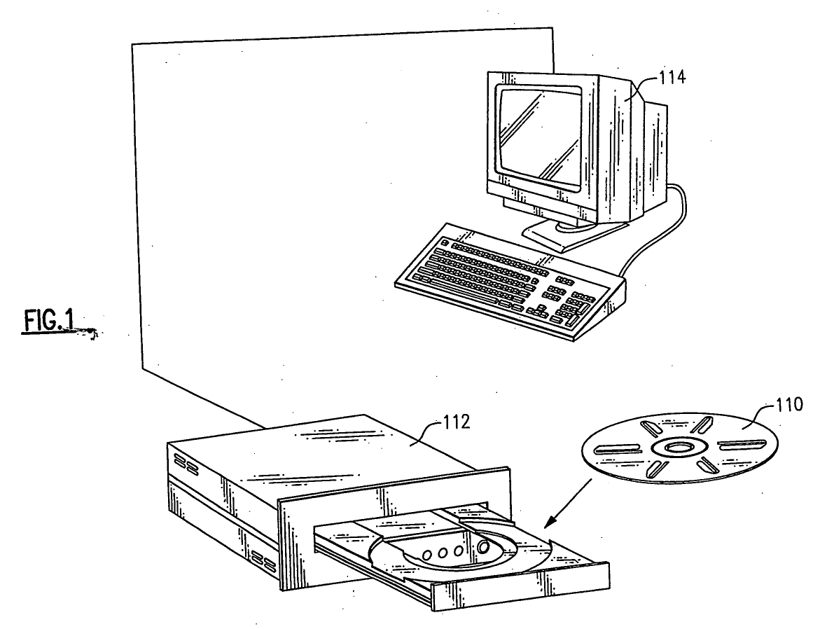

With reference now to FIG. 1, there is shown a perspective view of an optical bio-disc or medical CD 110 for use in an optical disc drive 112. Drive 112, in conjunction with software in the drive or associated with a separate computer, can cause images, graphs, or output data to be displayed on display monitor 114. As indicated below, there are different types of discs and drives that can be used. The disc drive can be in a unit separate from a controlling computer, or provided in a bay within a computer. The device can be made as portable as a laptop computer, and thus usable with battery power and in remote locations not generally served by advanced diagnostic equipment. The drive is preferably a conventional drive with minimal or no hardware modification, but can be a dedicated bio-disc or medical CD drive. Further details regarding these types of drive systems and related signal processing methods are disclosed in, for example, commonly assigned and co-pending U.S. patent application Ser. No. 09/378,878 entitled “Methods and Apparatus for Analyzing Operational and Non-operational Data Acquired from Optical Discs” filed Aug. 23, 1999; U.S. Provisional Patent Application Ser. No. 60/150,288 entitled “Methods and Apparatus for Optical Disc Data Acquisition Using Physical Synchronization Markers” filed Aug. 23, 1999; U.S. patent application Ser. No. 09/421,870 entitled “Trackable Optical Discs with Concurrently Readable Analyte Material” filed Oct. 26, 1999; U.S. patent application Ser. No. 09/643,106 entitled “Methods and Apparatus for Optical Disc Data Acquisition Using Physical Synchronization Markers” filed Aug. 21, 2000; U.S.; and U.S. patent application Ser. No. 10/043,688 entitled “Optical Disc Analysis System Including Related Methods For Biological and Medical Imaging” filed Jan. 10, 2002. These applications are herein incorporated by reference in their entirety.

Optical bio-disc 110 for use with embodiments of the present invention may have any suitable shape, diameter, or thickness, but preferably is implemented on a round disc with a diameter and a thickness similar to those of a compact disc (CD), a recordable CD (CD-R), CD-RW, a digital versatile disc (DVD), DVD-R, DVD-RW, or other standard optical disc format. The disc may include encoded information, preferably in a known format, for performing, controlling, and post-processing a test or assay, such as information for controlling the rotation rate and direction of the disc, timing for rotation, stopping and starting, delay periods, locations of samples, position of the light source, and power of the light source. Such encoded information is referred to generally here as operational information.

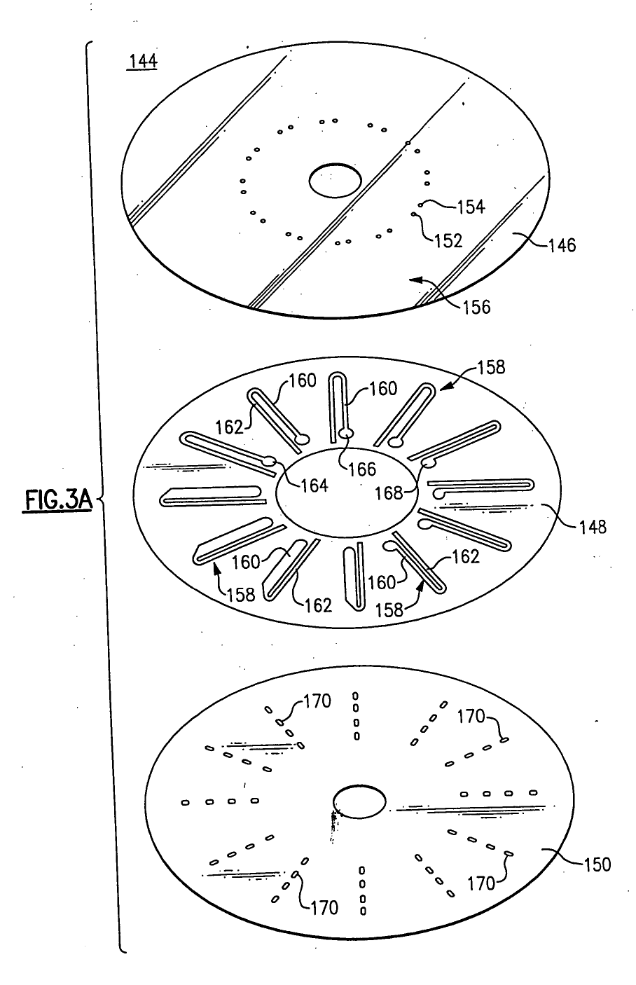

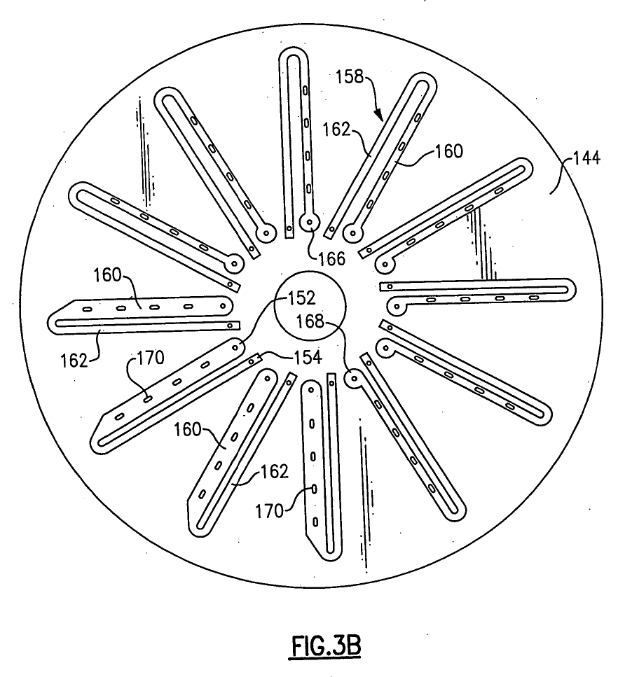

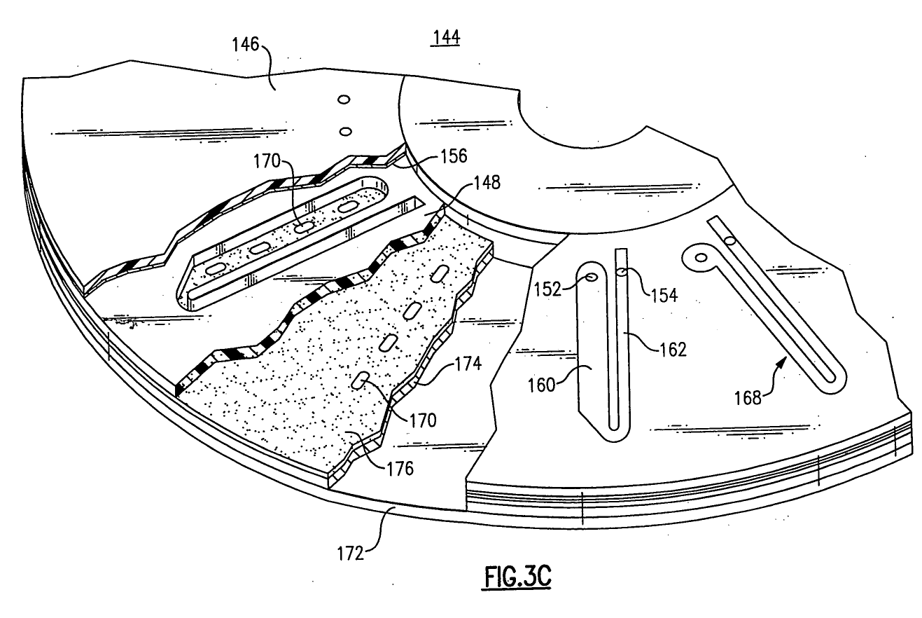

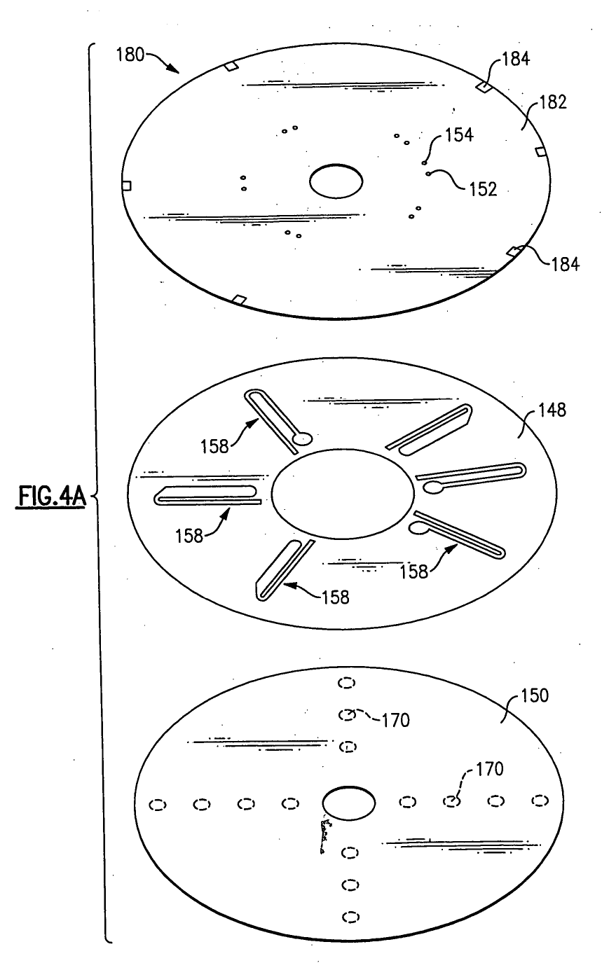

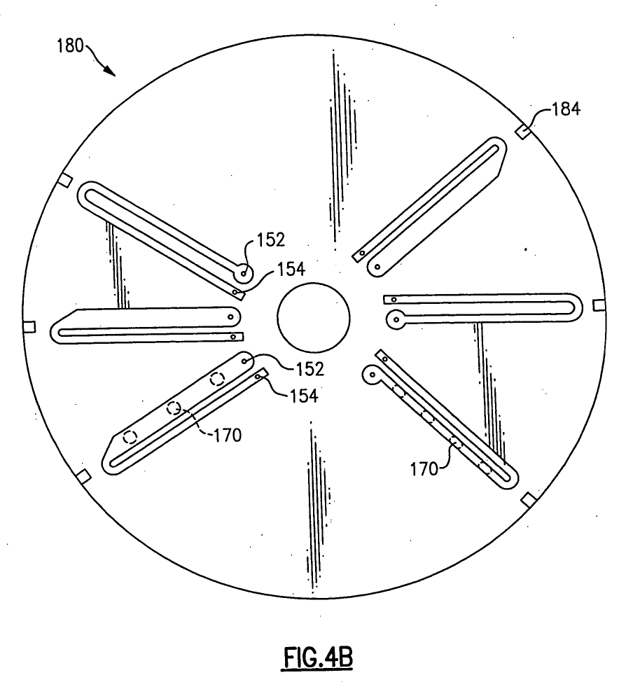

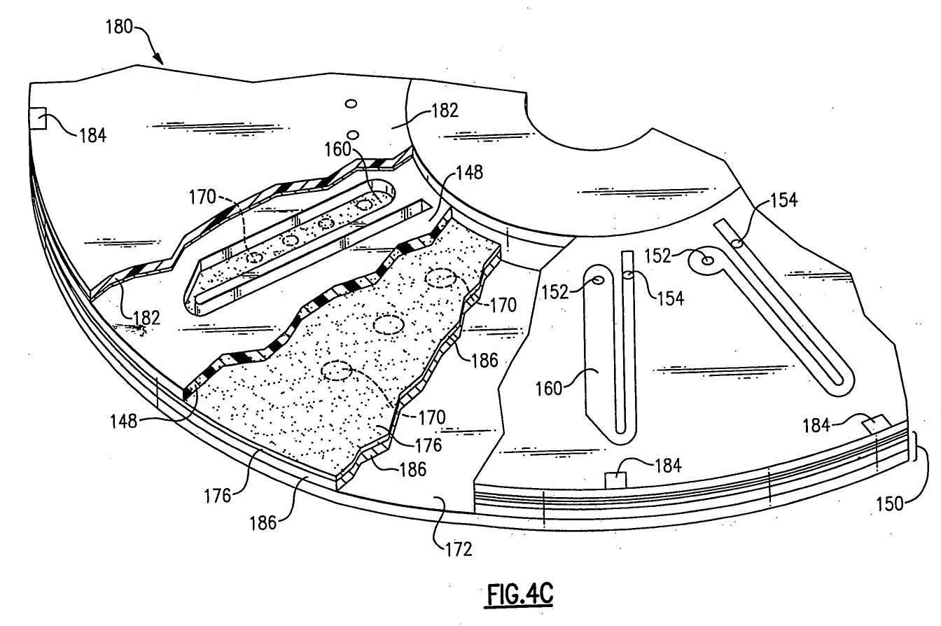

The disc may be a reflective disc, as shown in FIGS. 3A-3C, a transmissive disc, FIGS. 4A-4C, or some combination of reflective and transmissive. In a reflective disc, an incident light beam is focused onto the disc (typically at a reflective surface where information is encoded), reflected, and returned through optical elements to a detector on the same side of the disc as the light source. In a transmissive disc, light passes through the disc (or portions thereof) to a detector on the other side of the disc from the light source. In a transmissive portion of a disc, some light may also be reflected and detected as reflected light.

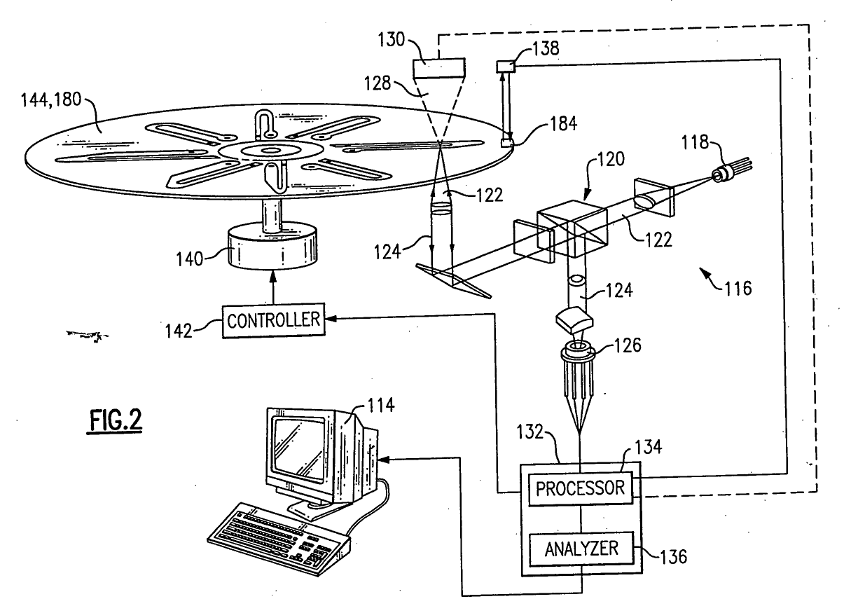

FIG. 2 shows an optical disc reader system 116. This system may be a conventional reader for CD, CD-R, DVD, or other known comparable format, a modified version of such a drive, or a completely distinct dedicated device. The basic components are a motor for rotating the disc, a light system for providing light, and a detection system for detecting light.

With reference now generally to FIGS. 2-4C, a light source 118 provides light to optical components 120 to produce an incident light beam 122. In the case of reflective disc 144, FIGS. 3A-3C, a return beam 124 is reflected from either reflective surface 156, 174, or 186, FIGS. 3C and 4C. Return beam 124 is provided back to optical components 120, and then to a bottom detector 126. In this type of disc, the return beam may carry operational information or other encoded data as well as characteristic information about the investigational feature or test sample under study.

For transmissive disc 180, FIGS. 4A-4C, some of the energy from the incident beam 122 will undergo a light/matter interaction with an investigational feature or test sample and then proceed through the disc as a transmitted beam 128 that is detected by a top detector 130. For a transmissive disc including a semi-reflective layer 186 (FIG. 4C) as the operational layer, some of the energy from the incident beam 122 will also reflect from the operational layer as return beam 124 which carries operational information or stored data. Optical components 120 can include a lens, a beam splitter, and a quarter wave plate that changes the polarization of the light beam so that the beam splitter directs a reflected beam through the lens to focus the reflected beam onto the detector. An astigmatic element, such as a cylindrical lens, may be provided between the beam splitter and detector to introduce astigmatism in the reflected light beam. The light source can be controllable to provide variable wavelengths and power levels over a desired range in response to data introduced by the user or read from the disc. This controllability is especially useful when it is desired to detect multiple different structures that fluoresce at different wavelengths.

Now with continuing reference to FIG. 2, it is shown that data from detector 126 and/or detector 130 is provided to a computer 132 including a processor 134 and an analyzer 136. An image or output results can then be provided to a monitor 114. Computer 132 can represent a desktop computer, programmable logic, or some other processing device, and also can include a connection (such as over the Internet) to other processing and/or storage devices. A drive motor 140 and a controller 142 are provided for controlling the rotation rate and direction or rotation of disc the 144 or 180. Controller 142 and the computer 132 with processor 134 can be in remote communication or implemented in the same computer. Methods and systems for reading such a disc are also shown in Gordon, U.S. Pat. No. 5,892,577, which is incorporated herein by reference.

The detector can be designed to detect all light that reaches the detector or, though its design or an external filter, only light at specific wavelengths. By making the detector controllable in terms of the detectable wavelength, beads or other structures that fluoresce at different wavelengths can be separately detected.

A hardware trigger sensor 138 may be used with either a reflective disc 144 or transmissive disc 180. Triggering sensor 138 provides a signal to computer 132 (or to some other electronics) to allow for the collection of data by processor 134 only when incident beam 122 is on a target zone or inspection area. Alternatively, software read from a disc can be used to control data collection by processor 134 independent of any physical marks on the disc. Such software or logical triggering is discussed in further detail in commonly assigned and co-pending U.S. Provisional Application Ser. No. 60/352,625 entitled “Logical Triggering Methods And Apparatus For Use With Optical Analysis Discs And Related Disc Drive Systems” filed Jan. 28, 2002, which is herein incorporated by reference in its entirety.

The substrate layer of the optical analysis disc may be impressed with a spiral track that starts at an innermost readable portion of the disc and then spirals out to an outermost readable portion of the disc. In a non-recordable CD, this track is made up of a series of embossed pits with varying length, each typically having a depth of approximately one-quarter the wavelength of the light that is used to read the disc. The varying lengths and spacing between the pits encode the operational data. The spiral groove of a recordable CD-like disc has a detectable dye rather than pits. This is where the operation information, such as the rotation rate, is recorded. Depending on the test, assay, or investigational protocol, the rotation rate may be variable with intervening or consecutive periods of acceleration, constant speed, and deceleration. These periods may be closely controlled both as to speed and time of rotation to provide, for example, mixing, agitation, or separation of fluids and suspensions with agents, reagents, antibodies, or other materials. Different optical analysis disc, medical CD, and bio-disc designs that may be utilized with the present invention, or readily adapted thereto, are disclosed, for example, in commonly assigned, copending U.S. patent application Ser. No. 09/999,274 entitled “Optical Bio-discs with Reflective Layers” filed on Nov. 15, 2001; U.S. patent application Ser. No. 10/005,313 entitled “Optical Discs for Measuring Analytes” filed Dec. 7, 2001; U.S. patent application Ser. No. 10/006,371 entitled “Methods for Detecting Analytes Using Optical Discs and Optical Disc Readers” filed Dec. 10, 2001; U.S. patent application Ser. No. 10/006,620 entitled “Multiple Data Layer Optical Discs for Detecting Analytes” filed Dec. 10, 2001; and U.S. patent application Ser. No. 10/006,619 entitled “Optical Disc Assemblies for Performing Assays” filed Dec. 10, 2001, which are all herein incorporated by reference in their entirety.

Numerous designs and configurations of an optical pickup and associated electronics may be used in the context of the embodiments of the present invention. Further details and alternative designs for compact discs and readers are described in Compact Disc Technology, by Nakajima and Ogawa, IOS Press, Inc. (1992); The Compact Disc Handbook, Digital Audio and Compact Disc Technology, by Baert et al. (eds.), Books Britain (1995); and CD-Rom Professional's CD-Recordable Handbook: The Complete Guide to Practical Desktop CD, Starrett et al. (eds.), ISBN 0910965188 (1996); all of which are incorporated herein in their entirety by reference.

The disc drive assembly is thus employed to rotate the disc, read and process any encoded operational information stored on the disc, and analyze the liquid, chemical, biological, or biochemical investigational features in an assay region of the disc. The disc drive assembly may be further utilized to write information to the disc either before, during, or after the material in the assay zone is analyzed by the read beam of the drive. In alternate embodiments, the disc drive assembly is implemented to deliver assay information through various possible interfaces such as via Ethernet to a user, over the Internet, to remote databases, or anywhere such information could be advantageously utilized. Further details relating to this type of disc drive interfacing are disclosed in commonly assigned copending U.S. patent application Ser. No. 09/986,078 entitled “Interactive System For Analyzing Biological Samples And Processing Related Information And The Use Thereof” filed Nov. 7, 2001, which is incorporated herein by reference in its entirety.

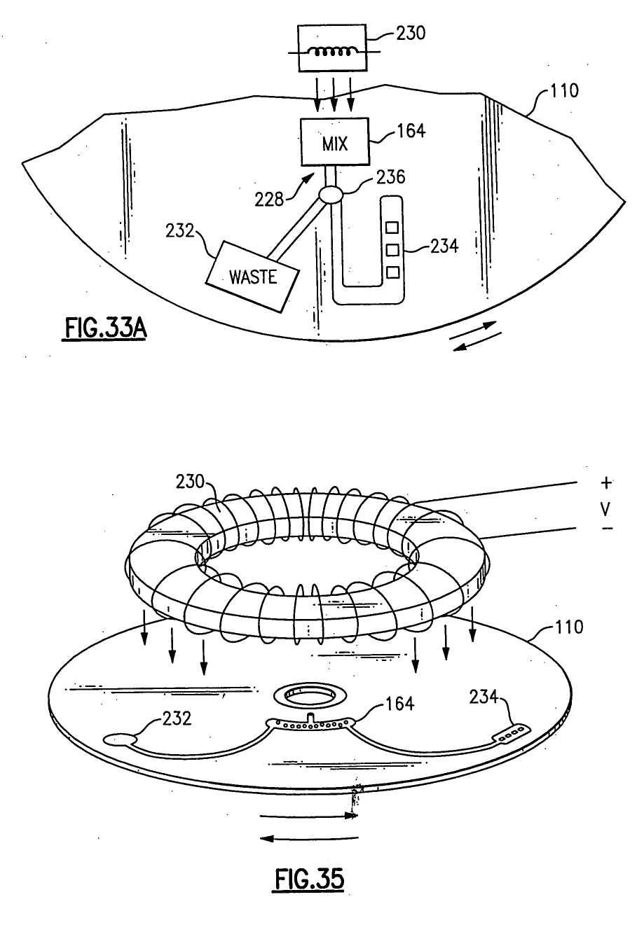

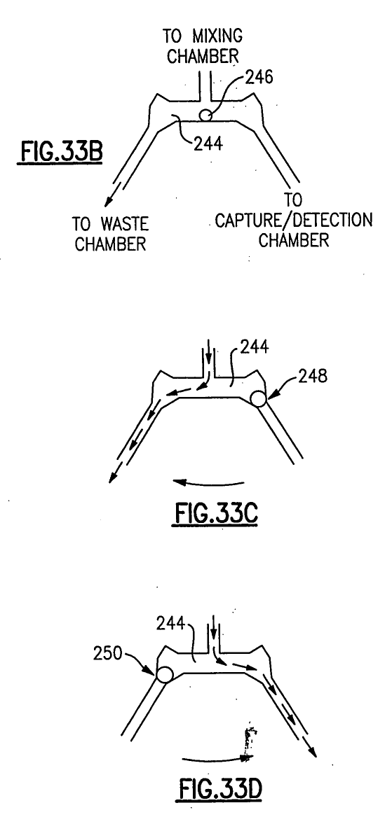

Referring now specifically to FIGS. 3A, 3B, and 3C, the reflective disc 144 is shown with a cap 146, a channel layer 148, and a substrate 150. The channel layer 148 may be formed by a thin-film adhesive member. Cap 146 has inlet ports 152 for receiving samples and vent ports 154. Cap 146 may be formed primarily from polycarbonate, and may be coated with a cap reflective layer 156 on the bottom thereof. Reflective layer 156 is preferably made from a metal such as aluminum or gold. Channel layer 148 defines fluidic circuits 158 by having desired shapes cut out from channel layer 148. Each fluidic circuit 158 preferably has a flow channel 160 and a return channel 162, and some have a mixing chamber 164. A mixing chamber 166 can be symmetrically formed relative to the flow channel 160, while an off-set mixing chamber 168 is formed to one side of the flow channel 160. Fluidic circuits 158 are rather simple in construction, but a fluidic circuit can include other channels and chambers, such as preparatory regions or a waste region as shown, for example, in commonly assigned U.S. Pat. No. 6,030,581 entitled “Laboratory in a Disk” which is incorporated herein by reference. These fluidic circuits can include valves and other fluid control structures such as those alternatively employed herein and discussed in further detail in connection with FIGS. 33A-33D, 34A-34C, 35, and 36A-36C. Channel layer 148 can include adhesives for bonding to the substrate and to the cap.

Substrate 150 has a plastic layer 172, and has target zones 170 formed as openings in a substrate reflective layer 174 deposited on the top of layer 172. In this embodiment, reflective layer 174, best illustrated in FIG. 3C, is used to encode operational information. Plastic layer 172 is preferably formed from polycarbonate. Target zones 170 may be formed by removing portions of the substrate reflective layer 174 in any desired shape, or by masking target zone areas before applying substrate reflective layer 174. The substrate reflective layer 174 is preferably formed from a metal, such as aluminum or gold, and can be configured with the rest of the substrate to encode operational information that is read with incident light, such as through a wobble groove or through an arrangement of pits. Light incident from under substrate 150 thus is reflected by layer 174, except at target zones 170, where it is reflected by layer 156. Target zones are where investigational features are detected. If the target zone is a location where an antibody, strand of DNA, or other material that can bind to a target is located, the target zone can be referred to as a capture zone.

With reference now particularly to FIG. 3C, optical disc 144 is cut away to illustrate a partial cross-sectional perspective view. An active layer 176 is formed over substrate reflective layer 174. Active layer 176 may generally be formed from nitrocellulose, polystyrene, polycarbonate, gold, activated glass, modified glass, or a modified polystyrene such as, for example, polystyrene-co-maleic anhydride. In this embodiment, channel layer 148 is situated over active layer 174.

In operation, samples can be introduced through inlet ports 152 of cap 146. When rotated, the sample moves outwardly from inlet port 152 along active layer 176. Through one of a number of biological or chemical reactions or processes, detectable features, referred to as investigational features, may be present in the target zones. Examples of such processes are shown in the incorporated U.S. Pat. No. 6,030,581 and in commonly assigned, co-pending U.S. patent application Ser. No. 09/988,728 entitled “Methods And Apparatus For Detecting And Quantifying Lymphocytes With Optical Biodiscs” filed Nov. 16, 2001; and U.S. patent application Ser. No. 10/035,836 entitled “Surface Assembly For Immobilizing DNA Capture Probes And Bead-Based Assay Including Optical Bio-Discs And Methods Relating Thereto” filed Dec. 21, 2001, both of which are herein incorporated by reference in their entireties.

The investigational features captured within the target zones, by the capture layer with a capture agent, may be designed to be located in the focal plane coplanar with reflective layer 174, where an incident beam is typically focused in conventional readers. Alternatively, the investigational features may be captured in a plane spaced away from the focal plane. The former configuration is referred to as a “proximal” type disc, and the latter a “distal” type disc.

Referring now to FIGS. 4A, 4B, and 4C, it is shown that one particular embodiment of the transmissive optical disc 180 includes a clear cap 182, a channel layer 148, and a substrate 150. The clear cap 182 includes inlet ports 152 and vent ports 154 and is preferably formed mainly from polycarbonate. Trigger marks 184 may be included on the cap 182. Channel layer 148 has fluidic circuits 158, which can have structure and use similar to those described in conjunction with FIGS. 3A, 3B, and 3C. Substrate 150 may include target zones 170, and preferably includes a polycarbonate layer 172. Substrate 150 may, but need not, have a thin semi-reflective layer 186 deposited on top of layer 172. Semi-reflective layer 186 is preferably significantly thinner than substrate reflective layer 174 on substrate 150 of reflective disc 144 (FIGS. 3A-3C). Semi-reflective layer 186 is preferably formed from a metal, such as aluminum or gold, but is sufficiently thin to allow a portion of an incident light beam to penetrate and pass through layer 186, while some of the incident light is reflected back. A gold film layer, for example, is 95% reflective at a thickness greater than about 700 Å, while the transmission of light through the gold film is about 50% transmissive at approximately 100 Å.

FIG. 4C is a cut-away perspective view of transmissive disc 180. The semi-reflective nature of layer 186 makes its entire surface potentially available for target zones, including virtual zones defined by trigger marks or encoded data patterns on the disc. Target zones 170 may also be formed by marking the designated area in the indicated shape or alternatively in any desired shape. Markings to indicate target zone 170 may be made on semi-reflective layer 186 or on a bottom portion of substrate 150 (under the disc). Target zones 170 may be created by silk screening ink onto semi-reflective layer 186.

An active layer 176 is applied over semi-reflective layer 186. Active layer 176 may be formed from the same materials as described above in conjunction with layer 176 (FIG. 3C) and serves substantially the same purpose when a sample is provided through an opening in disc 180 and the disc is rotated. In transmissive disc 180, there is no reflective layer, on the clear cap 182, comparable to reflective layer 156 in reflective disc 144 (FIG. 3C).

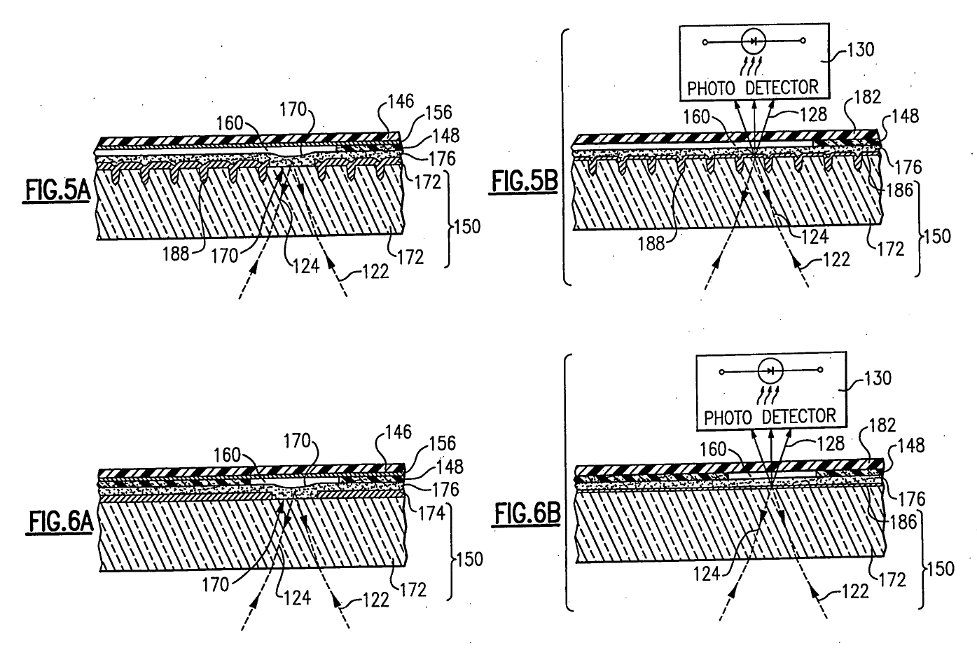

Referring now to FIG. 5A, there is shown a cross sectional view taken across the tracks of the reflective disc embodiment 144 (FIGS. 3A-3C) of the bio-disc 110 (FIG. 1) according to the present invention. As illustrated, this view is taken longitudinally along a radius and flow channel of the disc. FIG. 5A includes the substrate 150 which is composed of a plastic layer 172 and a substrate reflective layer 174. In this embodiment, the substrate 150 includes a series of grooves 188. The grooves 188 are in the form of a spiral extending from near the center of the disc toward the outer edge. The grooves 188 are implemented so that the interrogation or incident beam 122 may track along the spiral grooves 188 on the disc. This type of groove 188 is known as a “wobble groove”. The groove 188 is formed by a bottom portion having undulating or wavy side walls. A raised or elevated portion separates adjacent grooves 188 in the spiral. The reflective layer 174 applied over the grooves 188 in this embodiment is, as illustrated, conformal in nature. FIG. 5A also shows the active layer 176 applied over the reflective layer 174. As shown in FIG. 5A, the target zone 170 is formed by removing an area or portion of the reflective layer 174 at a desired location or, alternatively, by masking the desired area prior to applying the reflective layer 174. As further illustrated in FIG. 5A, the plastic adhesive member or channel layer 148 is applied over the active layer 176. FIG. 5A also shows the cap portion 146 and the reflective surface 156 associated therewith. Thus, when the cap portion 146 is applied to the plastic adhesive member 148 including the desired cut-out shapes, the flow channel 160 is thereby formed.

FIG. 5B is a cross sectional view, similar to that illustrated in FIG. 5A, taken across the tracks of the transmissive disc embodiment 180 (FIGS. 4A-4C) of the bio-disc 110 (FIG. 1) according to the present invention. This view is taken longitudinally along a radius and flow channel of the disc. FIG. 5B illustrates the substrate 150 that includes the thin semi-reflective layer 186. This thin semi-reflective layer 186 allows the incident or interrogation beam 122, from the light source 118 (FIG. 2), to penetrate and pass through the disc to be detected by the top detector 130, while some of the light is reflected back in the form of the return beam 124. The thickness of the thin semi-reflective layer 186 is determined by the minimum amount of reflected light required by the disc reader to maintain its tracking ability. The substrate 150 in this embodiment, like that discussed in FIG. 5A, includes the series of grooves 188. The grooves 188 in this embodiment are also preferably in the form of a spiral extending from near the center of the disc toward the outer edge. The grooves 188 are implemented so that the interrogation beam 122 may track along the spiral. FIG. 5B also shows the active layer 176 applied over the thin semi-reflective layer 186. As further illustrated in FIG. 5B, the plastic adhesive member or channel layer 148 is applied over the active layer 176. FIG. 5B also shows the clear cap 182. Thus, when the clear cap 182 is applied to the plastic adhesive member 148 including the desired cut-out shapes, the flow channel 160 is thereby formed and a part of the incident beam 122 is allowed to pass therethrough substantially unreflected. The amount of light that passes through can then be detected by the top detector 130.

FIG. 6A is a view similar to FIG. 5A but taken perpendicularly to a radius of the disc to illustrate the reflective disc and the initial refractive property thereof when observing the flow channel 160 from a radial perspective. In a parallel comparison manner, FIG. 6B is a similar view to FIG. 5B but taken perpendicularly to a radius of the disc to represent the transmissive disc and the initial refractive property thereof when observing the flow channel 160 from a radial perspective. Grooves 188 are not seen in FIGS. 5A and 5B since the sections are cut along the grooves 188. FIGS. 6A and 6B show the presence of the narrow flow channel 160 that is situated perpendicular to the grooves 188 in these embodiments. FIGS. 5A, 5B, 6A, and 6B show the entire thickness of the respective reflective and transmissive discs. In these views, the incident beam 122 is illustrated initially interacting with the substrate 150 which has refractive properties that change the path of the incident beam as shown to provide focusing of the beam 122 on the reflective layer 174 or the thin semi-reflective layer 186.

Assay Chemistries and Dual Bead Formation

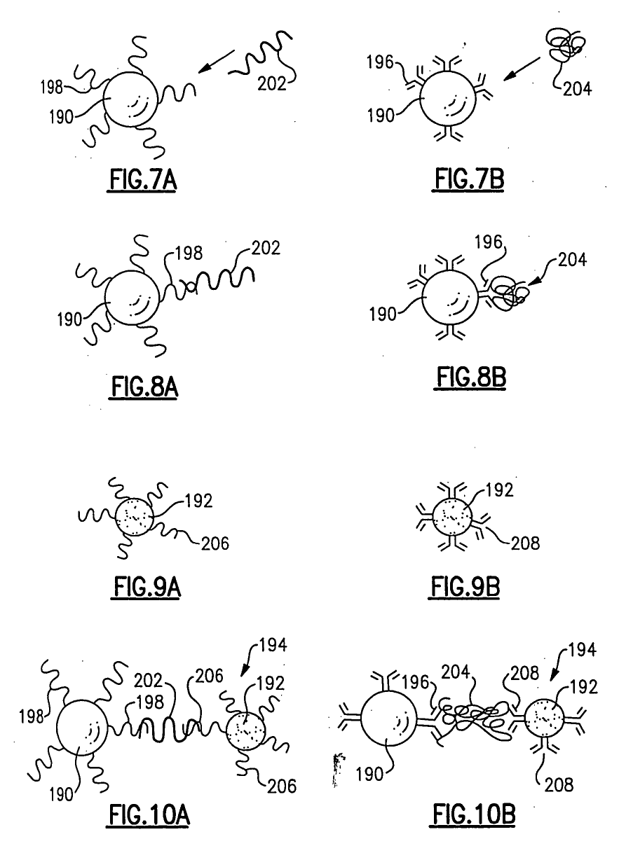

Referring now to FIGS. 7A-10A and 7B-10B, there is shown a capture bead 190, a reporter bead 192, and the formation of a dual bead complex 194. Capture bead 190 can be used in conjunction with a variety of different assays including biological assays such as immunoassays (FIGS. 7B-10B), molecular assays, and more specifically genetic assays (FIGS. 7A-10A). In the case of immunoassays, antibody transport probes 196 are conjugated onto the beads. Antibody transport probes 196 include proteins, such as antigens or antibodies, implemented to capture protein targets. In the case of molecular assays, oligonucleotide transport probes 198 are conjugated onto the beads. Oligonucleotide transport probes 198 include nucleic acids such as DNA or RNA implemented to capture genetic targets.

As shown in FIG. 7A, a target agent such as target DNA or RNA 202, obtained from a test sample, is added to a capture bead 190 coated with oligonucleotide transport probes 198. In this implementation, transport probes 198 are formed from desired sequences of nucleic acids. Aspects relating to DNA probe conjugation onto solid phase of this system of assays are discussed in further detail in commonly assigned and co-pending U.S. Provisional Application Ser. No. 60/278,685 entitled “Use of Double Stranded DNA for Attachment to Solid Phase to Reduce Non-Covalent Binding” filed Mar. 26, 2001. This application is herein incorporated by reference in its entirety.

As shown in FIG. 7B, a target agent such as target antigen 204 from a test sample is added to a capture bead 190 coated with antibody transport probes 196. In this alternate implementation, the transport probes 196 are formed from proteins such as antibodies.

Capture bead 190 has a characteristic that allows it to be isolated from a material suspension or solution. The capture bead may be selected based upon a desired size, and a preferred way to make it isolatable is for it to be magnetic.

FIG. 8A illustrates the binding of target DNA or RNA 202 to complementary transport probes 198 on capture bead 190 in the genetic assay implementation of the present invention. FIG. 8B shows an immunoassay version of FIG. 8A. Here transport probes 196 can alternatively include antibodies or antigens for binding to a target protein 204.

FIG. 9A shows a reporter bead 192 coated with oligonucleotide signal probes 206 complementary to target agent 202 (see FIG. 8A). Reporter bead 192 is selected based upon a desired size and the material properties for detection and reporting purposes. In one specific embodiment, a 2.1 micron polystyrene bead is employed. Signal probes 206 can be strands of DNA or RNA to capture target DNA or RNA.

FIG. 9B illustrates a reporter bead 192 coated with antibody signal probes 208 that bind to the target agent 204 as shown in FIG. 8B. Reporter bead 192 is selected based upon a desired size and the material properties for detection and reporting purposes. This may also preferably include a 2.1 micron polystyrene bead. Signal probes 208 can be antigens or antibodies implemented to capture protein or glycoportein targets.

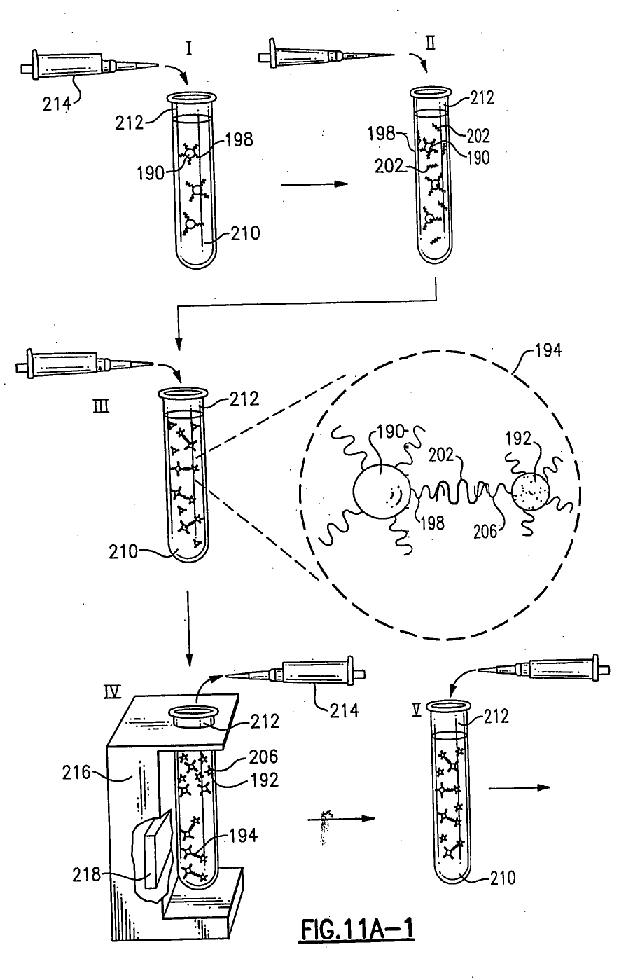

FIG. 10A is a pictorial representation of a dual bead complex 194 that can be formed from capture bead 190 with probe 198, target agent 202, and reporter bead 192 with probe 206. Probes 198 and 206 conjugated on capture bead 190 and reporter bead 192, respectively, have sequences complementary to the target agent 202, but not to each other. Further details regarding target agent detection and methods of reducing non-specific binding of target agents to beads are discussed in commonly assigned and co-pending U.S. Provisional Application Ser. No. 60/278,106 entitled “Dual Bead Assays Including Use of Restriction Enzymes to Reduce Non-Specific Binding” filed Mar. 23, 2001; and U.S. Provisional Application Ser. No. 60/278,110 entitled “Dual Bead Assays Including Use of Chemical Methods to Reduce Non-Specific Binding” also filed Mar. 23, 2001, which are both incorporated herein by reference in their entirety.

FIG. 10B is a pictorial representation of the immunoassay version of a dual bead complex 194 that can be formed from capture bead 190 with probe 196, target agent 204, and reporter bead 192 with probe 208. Probes 196 and 208 conjugated on capture bead 190 and reporter bead 192, respectively, only bind to the target agent 202, and not to each other.

In an alternative embodiment of the current system of assays, target agent binding efficiency and specificity may be enhanced by using a cleavable spacer that temporarily links the reporter bead 192 and capture bead 190. The dual bead complex formed by the cleavable spacer essentially places the transport probe and the signal probe in close proximity to each other thus allowing more efficient target binding to both probes. Once the target agent is bound to the probes, the spacer may then be cleaved permitting the bound target agent to retain the dual bead structure. The use of cleavable spacers in dual bead assay systems is disclosed in further detail in commonly assigned and co-pending U.S. Provisional Application Ser. No. 60/278,688 entitled “Dual Bead Assays Using Cleavable Spacers to Improve Specificity and Sensitivity” filed Mar. 26, 2001, which is herein incorporated in its entirety by reference.

With reference now to FIGS. 11A-1 and 11A-2, there is illustrated a method of preparing a molecular assay using a “single-step hybridization” technique to create dual bead complex structures in a solution according to one aspect of the present invention. This method includes eight principal steps identified consecutively as Steps I, II, III, IV, V, VI, VII, and VIII.

In Step I of this method, a number of capture beads 190 coated with oligonucleotide transport probes 198 are deposited into a test tube 212 containing a buffer solution 210. The number of capture beads 190 used in this method may be, for example, on the order of 10E+07 and each on the order of 1 micron or greater in diameter. Capture beads 190 are suspended in hybridization solution and are loaded into the test tube 212 by injection with pipette 214. The preferred hybridization solution is composed of 0.2M NaCl, 10 mM MgCl₂, 1 mM EDTA, 50 mM Tris-HCl, pH 7.5, and 5× Denhart's mix. A desirable hybridization temperature is 37 degrees Celsius. In a preliminary step in this embodiment, transport probes 198 are conjugated to 3 micron magnetic capture beads 190 by EDC conjugation. Further details regarding conjugation methods are disclosed in commonly assigned U.S. Provisional Application Ser. No. 60/271,922 entitled, “Methods for Attaching Capture DNA and Reporter DNA to Solid Phase Including Selection of Bead Types as Solid Phase” filed Feb. 27, 2001; and U.S. Provisional Application Ser. No. 60/277,854 entitled “Methods of Conjugation for Attaching Capture DNA and Reporter DNA to Solid Phase” filed Mar. 22, 2001, both of which are herein incorporated by reference in their entirety.

As shown in Step II, target DNA or RNA 202 is added to the solution. Oligonucleotide transport probes 198 are complementary to the DNA or RNA target agent 202. The target DNA or RNA 202 thus binds to the complementary sequences of transport probe 198 attached to the capture bead 190 as shown in FIG. 8A.

With reference now to Step III, there is added to the solution 210 reporter beads 192 coated with oligonucleotide signal probes 206. As also shown in FIGS. 9A and 10A, signal probes 206 are complementary to the target DNA or RNA 202. In one embodiment, signal probes 206, which are complementary to a portion of the target DNA or RNA 202, are conjugated to 2.1 micron fluorescent reporter beads 192. Signal probes 206 and transport probes 198 each have sequences that are complementary to the target DNA 202, but not complementary to each other. After adding reporter beads 192, the dual bead complex 194 is formed such that the target DNA 202 links capture bead 190 and reporter beads 192. With specific and thorough washing, there should be minimal non-specific binding between reporter bead 192 and capture bead 190. The target agent 202 and signal probe 206 are preferably allowed to hybridize for three to four hours at 37 degrees Celsius.

In this embodiment and others, it was found that intermittent mixing (i.e., periodically mixing and then stopping) produced greater yield of dual bead complex than continuous mixing during hybridization. Thus when this step is performed on-disc, the disc drive motor 140 and controller 142, FIG. 2, may be advantageously employed to periodically rotate the disc to achieve the desired intermittent mixing. This may be implemented in mixing protocols encoded on the disc that rotate the disc in one direction, then stop the disc, and thereafter rotate the disc again in the same direction in a prescribed manner with a preferred duty cycle of rotation and stop sessions. Alternatively, the encoded mixing protocol may rotate the disc in a first direction, then stop the disc, and thereafter rotate the disc again in the opposite direction with a preferred duty cycle of rotation, stop, and reverse rotation sessions. These features of the present invention are discussed in further detail in connection with FIGS. 33A and 35.

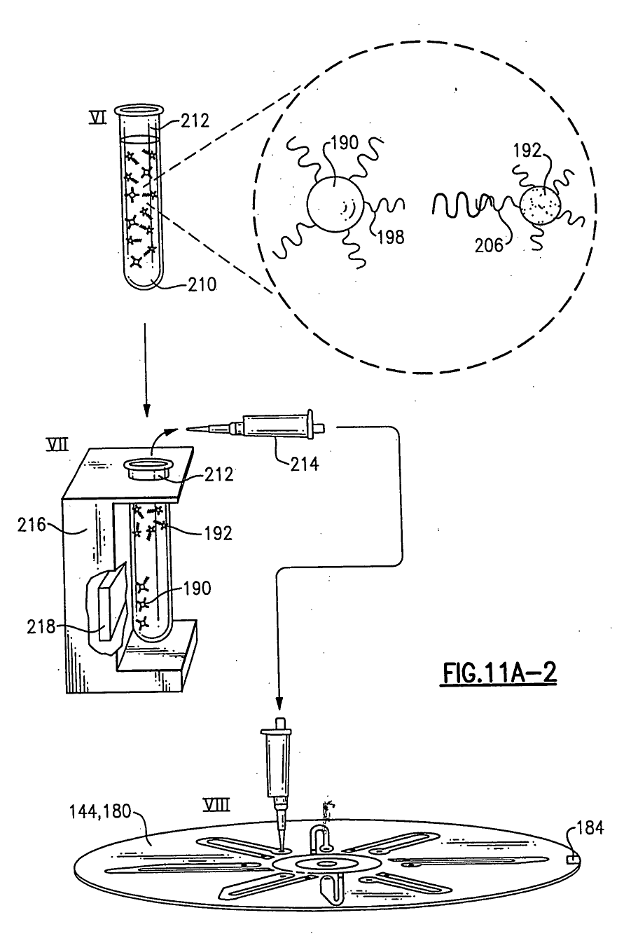

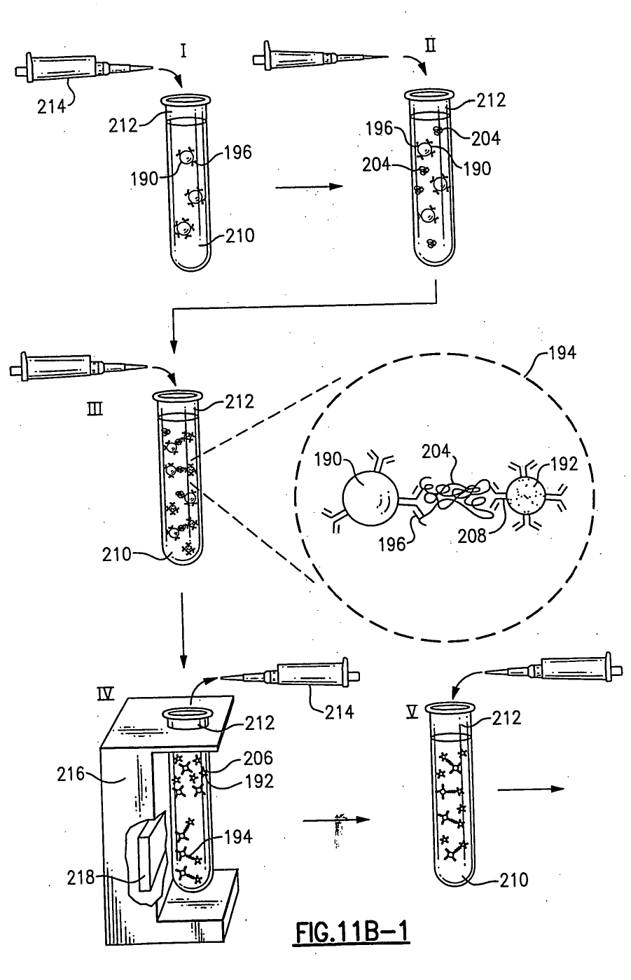

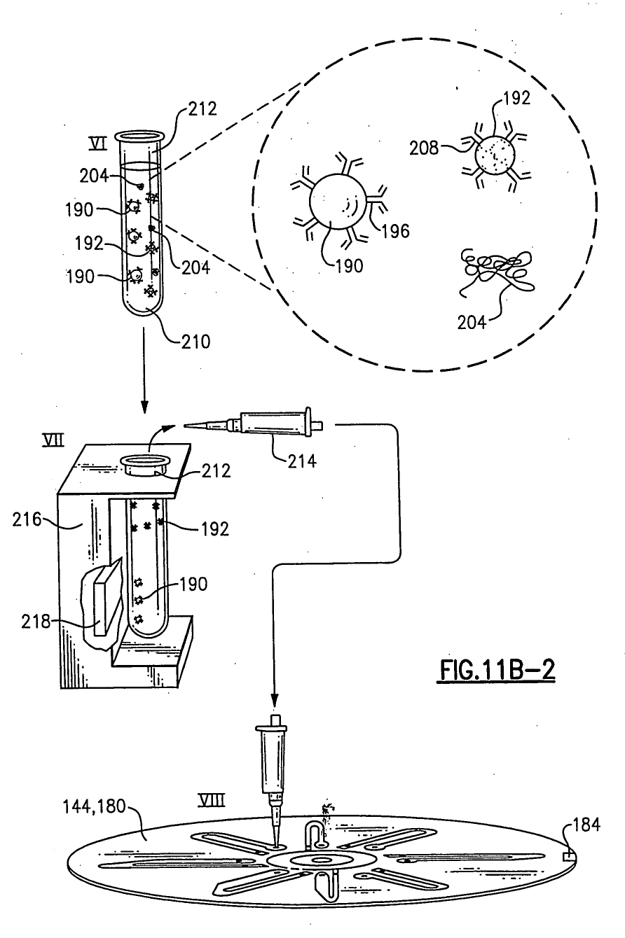

As next shown in Step IV of FIG. 11A-1, after hybridization, the dual bead complex 194 is separated from unbound reporter beads in the solution. The solution can be exposed to a magnetic field to capture the dual bead complex structures 194 using the magnetic properties of capture bead 190. The magnetic field can be encapsulated in a magnetic test tube rack 216 with a built-in magnet 218, which can be permanent or electromagnetic to draw out the magnetic beads and remove any unbound reporter beads in the suspension. Note that capture beads not bound to reporter beads will also be isolated. Alternatively, this magnetic removal step may be performed on-disc as shown in FIGS. 33A, 35, and 36A-36C.

The purification process illustrated in Step IV includes the removal of supernatant containing free-floating particles. Wash buffer is added into the test tube and the bead solution is mixed well. The preferred wash buffer for the one step assay consists of 145 mM NaCl, 50 mM Tris, pH 7.5, 0.1% SDS, 0.05% Tween, 0.25% NFDM, and 10 mM EDTA. Most of the unbound reporter beads 182, free-floating DNA, and non-specifically bound particles are agitated and removed from the supernatant. The dual bead complex can form a matrix of capture beads, target sequences, and reporter beads, wherein the wash process can further assist in the extraction of free floating particles trapped in the lattice structure of overlapping dual bead particles. Further details relating to other aspects associated with methods of decreasing non-specific binding of reporter beads to capture beads are disclosed in, for example, commonly assigned U.S. Provisional Application Ser. No. 60/272,134 entitled “Reduction of Non-Specific Binding in Dual Bead Assays by Selection of Bead Type and Bead Treatment” filed Feb. 28, 2001; and U.S. Provisional Application Ser. No. 60/275,006 entitled “Reduction of Non-Specific Binding in Dual Bead Assays by Selection of Buffer Conditions and Wash Conditions” filed Mar. 12, 2001. Both of these applications are herein incorporated by reference in their entirety.

The next step in FIG. 11A-1 is Step V. In this step, once the dual bead complex has been washed approximately 3-5 times with wash buffer solution, restriction enzymes, urea, acids (preferably strong acids), or bases (preferably strong bases) may be added to the dual bead solution, as illustrated. The dual bead complexes are thus dissociated by the actions of these dissociation agents thereby releasing the reporter beads 192 from the capture beads 190 as shown in FIG. 11A-2, Step VI.

After the dissociation of the dual bead structure, the capture beads 190 are separated from the, now unbound, reporter beads 192 in the solution, as shown in Step VII. The solution can be exposed to a magnetic field to capture the magnetic capture beads 190. The magnetic field can be encapsulated in a magnetic test tube rack 216 with a built-in magnet 218, which can be permanent or electromagnetic to draw out the magnetic beads and remove any unbound reporter beads in the suspension. Note that non-dissociated dual bead complexes not separated during Step VI will also be removed from the solution. During step VII, the supernatant containing the released reporter beads are collected using a pipette 214. The assay mixture may then be loaded into the disc 144 or 180 and analyzed using an optical bio-disc or medical CD reader, as illustrated in Step VIII. Either a transmissive bio-disc 180 or a reflective bio-disc 144 may be used to analyze the reporter beads. Details relating to the reflective and transmissive optical bio-discs are discussed above in conjunction with FIGS. 3A-3C and 4A-4C, respectively. The optical bio-disc reader and other alternative disc readers that may be used to analyze optical bio-discs are described in detail above in connection with FIGS. 1 and 2. If the reporter beads are fluorescent, the reporter beads isolated in Step VII may also be quantified using a fluorimeter or any similar fluorescent type analyzer including fluorescent optical disc readers.

FIGS. 11B-1 and 11B-2 taken together illustrate an immunoassay using a “single-step antigen binding” method, similar to that in FIGS. 11A-1 and 11A-2, to create dual bead complex structures in a solution. This method similarly includes eight principal steps. These steps are respectively identified as Steps I, II, III, IV, V, VI, VII, and VIII in FIGS. 11B-1 and 11B-2.

As shown in Step I, FIG. 1B-1, capture beads 190, e.g., on the order of 10E+07 in number and each on the order of 1 micron or above in diameter, which are coated with antibody transport probes 196 are added to a buffer solution 210. This solution may be the same as that employed in the method shown in FIGS. 11A-1 and 11A-2 or alternatively may be specifically prepared for use with immunochemical assays. The antibody transport probes 196 have a specific affinity for the target antigen 204. The transport probes 196 bind specifically to epitopes within the target antigen 204 as also shown in FIG. 8B. In one embodiment, antibody transport probes 196 which have an affinity for a portion of the target antigen may be conjugated to 3 micron magnetic capture beads 190 via EDC conjugation. Alternatively, conjugation of the transport probes 196 to the capture bead 190 may be achieved by passive adsorption.

With reference now to Step II shown in FIG. 11B-1, the target antigen 204 is added to the solution. The target antigen 204 binds to the antibody transport probe 196 attached to the capture bead 190 as also shown in FIG. 8B.

As illustrated in Step III, reporter beads 192 coated with antibody signal probes 208 are added to the solution. Antibody signal probes 208 specifically binds to the epitopes on target antigen 204 as also represented in FIGS. 9B and 10B. In one embodiment, signal probes 208 are conjugated to 2.1 micron fluorescent reporter beads 192. Signal probes 208 and transport probes 196 each bind to specific epitopes on the target antigen, but not to each other. After adding reporter beads 192, the dual bead complex 194 is formed such that the target antigen 204 links capture bead 190 and reporter bead 192. With specific and thorough washing, there should be minimal non-specific binding between reporter bead 192 and capture bead 190.

In Step IV, after the binding in Step III, the dual bead complex 194 is separated from unbound reporter beads in the solution. The solution can be exposed to a magnetic field to capture the dual bead complex structures 194 using the magnetic properties of capture bead 190. The magnetic field can be encapsulated in a magnetic test tube rack 216 with a built-in magnet 218, which can be permanent or electromagnetic to draw out the magnetic beads and remove any unbound reporter beads in the suspension. Note that capture beads not bound to reporter beads will also be isolated. Alternatively, as indicated above, this magnetic removal step may also be performed on-disc as shown in FIGS. 33A, 35, and 36A-36C.

The purification process of Step IV includes the removal of supernatant containing free-floating particles. Wash buffer is added into the test tube and the bead solution is mixed well. Most of the unbound reporter beads 182, free-floating protein samples, and non-specifically bound particles are agitated and removed from the supernatant. The dual bead complex can form a matrix of capture beads, target antigen, and reporter beads, wherein the wash process can further assist in the extraction of free floating particles trapped in the lattice structure of overlapping dual bead particles.

The next step in FIG. 11B-1 is Step V. In this step, once the dual bead complex has been washed approximately 3-5 times with wash buffer solution. Urea, acids, or bases may be added to the dual bead solution as dissociation agents, using a pipette 214 as illustrated. The acids or bases employed herein are preferably strong acids or bases, respectively. The dual bead complexes are thus dissociated by the actions of these dissociation agents thereby releasing the reporter beads 192 from the capture beads 190 as shown in FIG. 11B-2, Step VI.

After the dissociation of the dual bead structure, the capture beads 190 are separated from the, now unbound, reporter beads 192 in the solution, as shown in Step VII. The solution can be exposed to a magnetic field, either on-disc or off-disc, to capture the magnetic capture beads 190. In the preparatory off-disc method shown here, the magnetic field can be encapsulated in a magnetic test tube rack 216 with a built-in magnet 218, which can be permanent or electromagnetic to draw out the magnetic beads and remove any unbound reporter beads in the suspension. Note that non-dissociated dual bead complexes not separated during Step VI will also be removed from the solution. In Step VII, the supernatant containing the released reporter beads are collected using a pipette 214. The assay mixture may then be loaded directly into the disc and analyzed using an optical bio-disc reader, as illustrated in Step VIII. Either a transmissive bio-disc 180 or a reflective bio-disc 144 may be used to analyze the reporter beads. Details relating to the reflective and transmissive optical bio-discs are discussed in detail in connection with FIGS. 3A-3C and 4A-4C, respectively. The optical bio-disc reader and other alternative disc readers that may be used to analyze optical bio-discs are described in detail above with reference to FIGS. 1 and 2. If the reporter beads are fluorescent, the reporter beads isolated in Step VII may also be quantified using a fluorimeter or any similar fluorescent type analyzer including fluorescent optical disc readers.

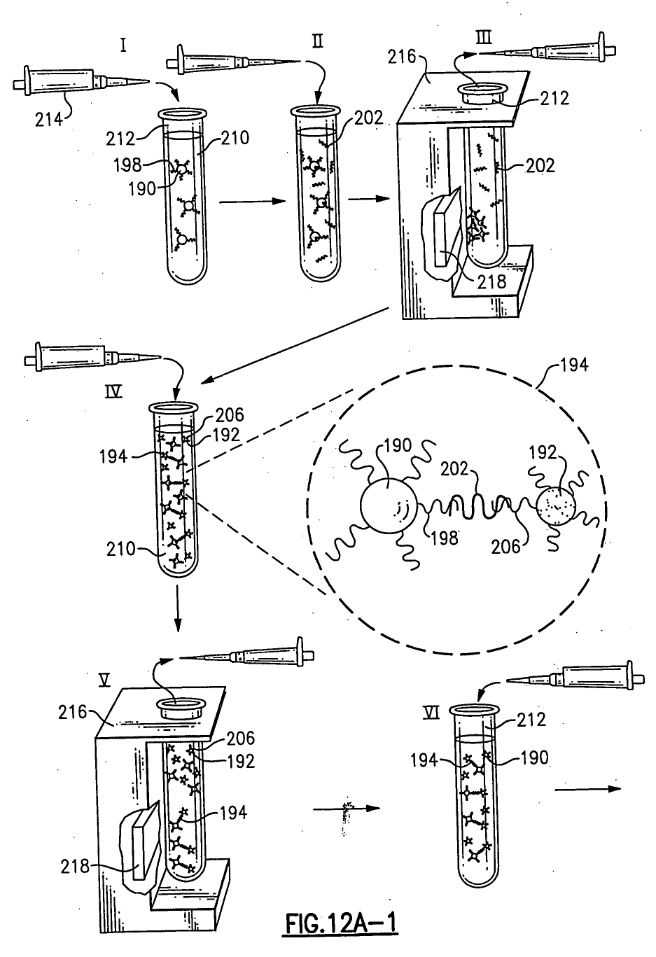

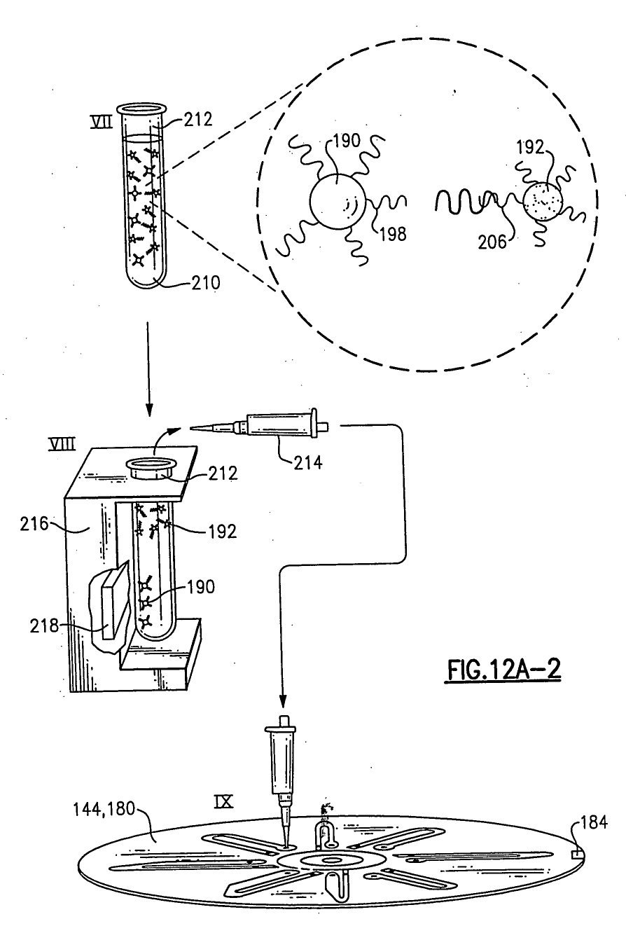

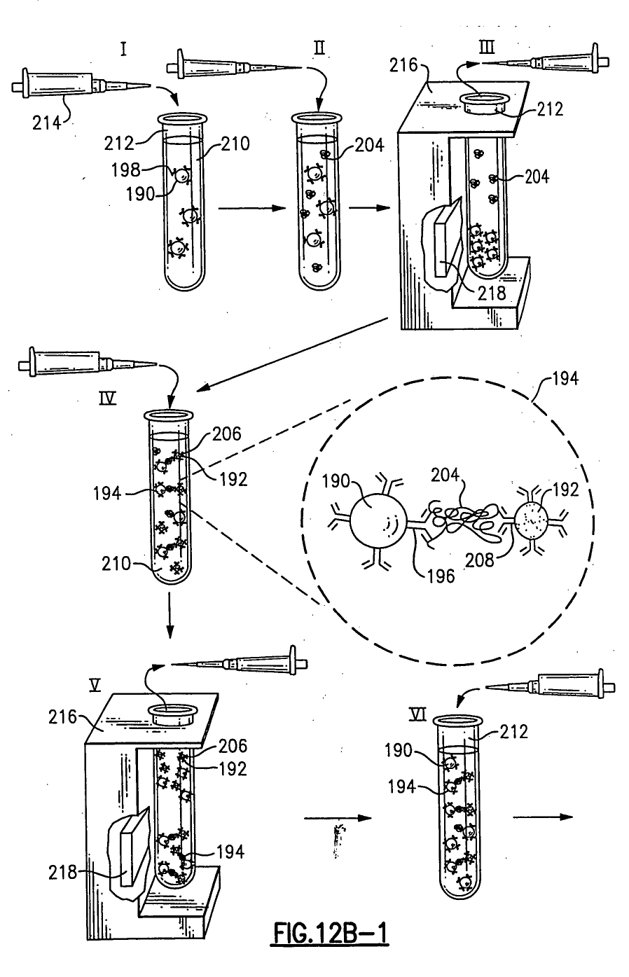

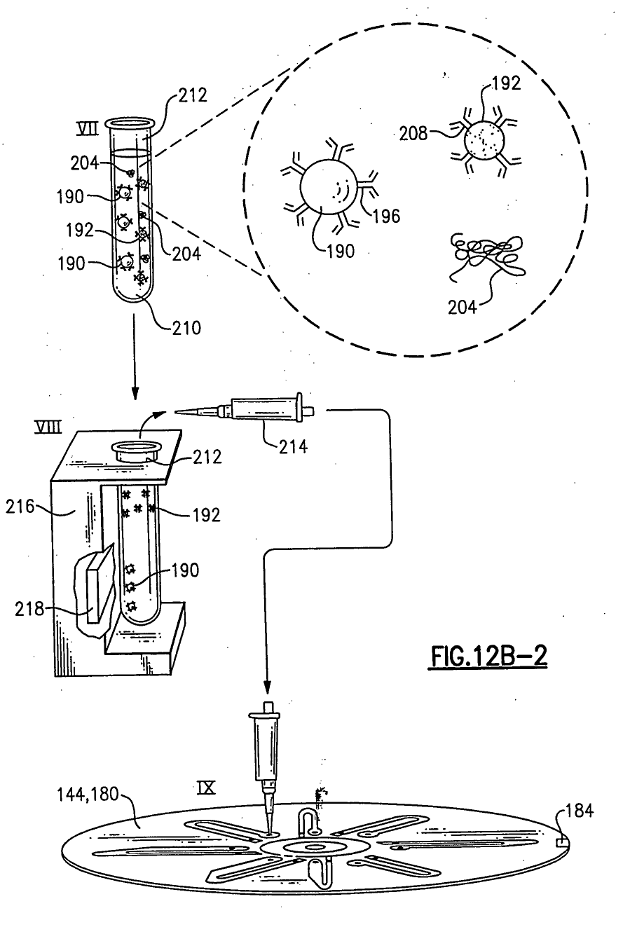

FIGS. 12A-1 and 12A-2 taken together show an alternative genetic assay method referred to here as a “two-step hybridization”. This method has nine principal steps directed to creating the dual bead complex. Generally, capture beads are coated with oligonucleotide transport probes 198 complementary to DNA or RNA target agent and placed into a buffer solution. In this embodiment, transport probes which are complementary to a portion of target agent are conjugated to 3 micron magnetic capture beads via EDC conjugation. Other type of conjugation of the oligonucleotide transport probes to a solid phase may be utilized. These include, for example, passive adsorption or use of streptavidin-biotin interactions. The nine main steps according to this method of the present invention are consecutively identified as Steps I, II, III, IV, V, and VI in FIG. 12A-1, and Steps VII, VIII, and IX in FIG. 12A-2.

More specifically now with reference to Step I shown in FIG. 12A-1, capture beads 190, suspended in hybridization solution, are loaded from the pipette 214 into the test tube 212. The preferred hybridization solution is composed of 0.2M NaCl, 10 mM MgCl₂, 1 mM EDTA, 50 mM Tris-HCl, pH 7.5, and 5× Denhart's mix. A desirable hybridization temperature is 37 degrees Celsius.

In Step II, target DNA or RNA 202 is added to the solution and binds to the complementary sequences of transport probe 198 attached to capture bead 190. In one specific embodiment of this method, target agent 202 and the transport probe 198 are allowed to hybridize for 2 to 3 hours at 37 degrees Celsius. Sufficient hybridization, however, may be achieved within 30 minutes at room temperature. At higher temperatures, hybridization may be achieved substantially instantaneously.

As next shown in Step III, target agents 202 bound to the capture beads are separated from unbound species in solution by exposing the solution to a magnetic field to isolate bound target sequences by using the magnetic properties of the capture bead 190. The magnetic field can be enclosed in a magnetic test tube rack 216 with a built-in magnet permanent 218 or electromagnet to draw out the magnetic beads and remove any unbound target DNA 202 free-floating in the suspension via pipette extraction of the solution. As with the above methods, in the on-disc counterpart hereto, this magnetic removal step may be performed as shown in FIGS. 33A, 35, and 36A-36C. A wash buffer is added and the separation process can be repeated. The preferred wash buffer after the transport probes 198 and target DNA 202 hybridize, consists of 145 mM NaCl, 50 mM Tris, pH 7.5, and 0.05% Tween. Hybridization methods and techniques for decreasing non-specific binding of target agents to beads are further disclosed in commonly assigned and co-pending U.S. Provisional Application Ser. No. 60/278,691 entitled “Reduction of Non-Specific Binding of Dual Bead Assays by Use of Blocking Agents” filed Mar. 26, 2001. This application is herein incorporated by reference in its entirety.

Referring now to Step IV illustrated in FIG. 12A-1, reporter beads 192 are added to the solution as discussed in conjunction with the method shown in FIG. 11A-1. Reporter beads 192 are coated with signal probes 206 that are complementary to target agent 202. In one particular embodiment of this method, signal probes 206, which are complementary to a portion of target agent 202, are conjugated to 2.1 micron fluorescent reporter beads 192. Signal probes 206 and transport probes 198 each have sequences that are complementary to target agent 202, but not complementary to each other. After the addition of reporter beads 192, the dual bead complex structures 190 are formed. As would be readily apparent to one of skill in the art, the dual bead complex structures are formed only if the target agent of interest is present. In this formation, target agent 202 links magnetic capture bead 190 and reporter bead 192. Using the preferred buffer solution, with specific and thorough washing, there is minimal non-specific binding between the reporter beads and the capture beads. Target agent 202 and signal probe 206 are preferably allowed to hybridize for 2-3 hours at 37 degrees Celsius. As with Step II discussed above, sufficient hybridization may be achieved within 30 minutes at room temperature. At higher temperatures, the hybridization taking place in this step may also be achieved substantially instantaneously.

With reference now to Step V shown in FIG. 12A-1, after the hybridization in Step IV, the dual bead complex 194 is separated from unbound species in solution. The solution is again exposed to a magnetic field to isolate the dual bead complex 194 using the magnetic properties of the capture bead 190. Note again that the isolate will include capture beads not bound to reporter beads. As with Step III above in the on-disc counterpart hereto, this magnetic separation step may be performed as shown in FIGS. 33A, 35, and 36A-36C.

A purification process to remove supernatant containing free-floating particles includes adding wash buffer into the test tube and mixing the bead solution well. The preferred wash buffer for the two-step assay consists of 145 mM NaCl, 50 mM Tris, pH 7.5, 0.1% SDS, 0.05% Tween, 0.25% NFDM, and 10 mM EDTA. Most unbound reporter beads, free-floating DNA, and non-specifically bound particles are agitated and removed from the supernatant. The dual bead complex can form a matrix of capture beads, target agents, and reporter beads, wherein the wash process can further assist in the extraction of free floating particles trapped in the lattice structure of overlapping dual bead particles. Other related aspects directed to reduction of non-specific binding between reporter bead, target agent, and capture bead are disclosed in, for example, commonly assigned U.S. Provisional Application Ser. No. 60/272,243 entitled “Mixing Methods to Reduce Non-Specific Binding in Dual Bead Assays” filed Feb. 28, 2001; and U.S. Provisional Application Ser. No. 60/272,485 entitled “Dual Bead Assays Including Linkers to Reduce Non-Specific Binding” filed Mar. 1, 2001, which are incorporated herein in their entirety.

The next step shown in FIG. 12A-1 is Step VI. In this step, once the dual bead complex 194 has been washed approximately 3-5 times with wash buffer solution, the assay mixture is loaded into the disc and analyzed. Alternatively, during this step, the oligonucleotide signal and transport probes may be ligated to prevent breakdown of the dual bead complex during the disc analysis and signal detection processes. Further details regarding probe ligation methods are disclosed in commonly assigned and co-pending U.S. Provisional Application Ser. No. 60/278,694 entitled “Improved Dual Bead Assays Using Ligation” filed Mar. 26, 2001, which is herein incorporated in its entirety by reference. In yet another embodiment of the present invention, restriction enzymes, urea, acids, or bases may be added using a pipette 214 to the dual bead solution, as illustrated in Step VI of FIG. 12A-1. The dual bead complexes are dissociated by the actions of these dissociation agents thus releasing the reporter beads 192 from the capture beads 190 as shown in FIG. 12A-2, Step VII. The acids or bases utilized herein are preferably strong acids or bases, respectively.

After the dissociation of the dual bead structure, the capture beads 190 are separated from the, now unbound, reporter beads 192 in the solution, as shown in Step VIII. The solution can be exposed to a magnetic field to capture the magnetic capture beads 190. The magnetic field can be encapsulated in a magnetic test tube rack 216 with a built-in magnet 218, which can be permanent or electromagnetic to draw out the magnetic beads and remove any unbound reporter beads in the suspension. Note that non-dissociated dual bead complexes not separated during Step VIII will also be removed from the solution. During Step VIII, the supernatant containing the released reporter beads are collected using a pipette 214. The assay mixture may then be loaded into the disc and analyzed using an optical bio-disc reader, as illustrated in Step IX. Either a transmissive bio-disc 180 or a reflective bio-disc 144 may be used to analyze the reporter beads. Details relating to the reflective and transmissive optical bio-discs are discussed in detail in conjunction with FIGS. 3A-3C and 4A-4C, respectively. The optical bio-disc reader and other alternative disc readers that may be used to analyze optical bio-discs are described in detail above with reference to FIGS. 1 and 2. If the reporter beads are fluorescent, the reporter beads isolated in Step VIII may also be quantified using a fluorimeter or any similar fluorescent type analyzer including fluorescent optical disc readers. Experiments performed using dissociation agents to release the reporter beads from the dual bead complex are described in detail below in Examples 3 and 4.

In accordance with another aspect of this invention, FIGS. 12B-1 and 12B-2 taken together show an immunoassay method, similar to those discussed in connection with FIGS. 11B-1, 11B-2, and following the techniques of the genetic assay in FIGS. 12A-1, and 12A-2. This method is also referred to here as a “two-step binding” to create the dual bead complex in an immunochemical assay. As with the method shown in FIGS. 12A-1 and 12A-2, this method includes nine main steps. In general, capture beads coated with antibody transport probes which specifically binds to epitopes on target antigen are placed into a buffer solution. In one specific embodiment, antibody transport probes are conjugated to 3 micron magnetic capture beads. Different sized magnetic capture beads may be employed depending on the type of disc drive and disc assembly utilized to perform the assay. The nine main steps according to this alternative method of the invention are respectively identified as Steps I, II, III, IV, V and VI in FIG. 12B-1 and Steps VII, VIII, and IX in FIG. 12B-2.

With specific reference now to Step I shown in FIG. 12B-1, capture beads 190, suspended in buffer 210 solution, are loaded into a test tube 212 via injection from pipette 214.

In Step II, target antigen 204 is added to the solution and binds to the antibody transport probe 196 attached to capture bead 190. Target antigen 204 and the transport probe 196 are preferably allowed to bind for 2 to 3 hours at 37 degrees Celsius. Shorter binding times are also possible.

As shown in Step III, target antigen 204 bound to the capture beads 190 are separated from unbound species in solution by exposing the solution to a magnetic field to isolate bound target proteins or glycoproteins by using the magnetic properties of the capture bead 190. The magnetic field can be enclosed in a magnetic test tube rack 216 with a built-in magnet permanent 218 or electromagnet to draw out the magnetic beads and remove any unbound target antigen 204 free-floating in the suspension via pipette extraction of the solution. A wash buffer is added and the separation process can be repeated.

As next illustrated in Step IV, reporter beads 192 are added to the solution as discussed in conjunction with the method shown in FIG. 11B-1. Reporter beads 192 are coated with signal probes 208 that have an affinity for the target antigen 204. In one particular embodiment of this two-step immunochemical assay, signal probes 208, which bind specifically to a portion of target agent 204, are conjugated to 2.1 micron fluorescent reporter beads 192. Signal probes 208 and transport probes 196 each bind to specific epitopes on the target agent 204, but do not bind to each other. After the addition of reporter beads 192, the dual bead complex structures 190 are formed. As would be readily apparent to those skilled in the art, these dual bead complex structures are formed only if the target antigen of interest is present. In this formation, target antigen 204 links magnetic capture bead 190 and reporter bead 192. Using the preferred buffer solution, with specific and thorough washing, there is minimal non-specific binding between the reporter beads and the capture beads. Target antigen 204 and signal probe 208 are allowed to hybridize for 2-3 hours at 37 degrees Celsius. As with Step II discussed above, sufficient binding may be achieved within 30 minutes at room temperature. In the case of immunoassays temperatures higher than 37 degrees Celsius are not preferred because the proteins will denature.

Turning next to Step V as illustrated in FIG. 12B-1, after the binding shown in Step IV, the dual bead complex 194 is separated from unbound species in solution. This is achieved by exposing the solution to a magnetic field to isolate the dual bead complex 194 using the magnetic properties of the capture bead 190 as shown. Note again that the isolate will include capture beads not bound to reporter beads.

A purification process to remove supernatant containing free-floating particles includes adding wash buffer into the test tube and mixing the bead solution well. Most unbound reporter beads, free-floating proteins, and non-specifically bound particles are agitated and removed from the supernatant. The dual bead complex can form a matrix of capture beads, target agents, and reporter beads, wherein the wash process can further assist in the extraction of free floating particles trapped in the lattice structure of overlapping dual bead particles.

The last step shown in FIG. 12B-1 is Step VI. In this step, once the dual bead complex 194 has been washed approximately 3-5 times with wash buffer solution, urea, acids (preferably strong acids), or bases (preferably strong bases) may be added to the dual bead solution using a pipette 214, as illustrated in Step VI of FIG. 12B-1. The dual bead complexes are dissociated by the actions of these dissociation agents thus releasing the reporter beads 192 from the capture beads 190 as shown next in Step VII of FIG. 12B-2.

After the dissociation of the dual bead structure, the capture beads 190 are separated from the, now unbound, reporter beads 192 in the solution, as shown in Step VIII of FIG. 12B-2. The solution can be exposed to a magnetic field to capture the magnetic capture beads 190. The magnetic field can be encapsulated in a magnetic test tube rack 216 with a built-in magnet 218, which can be permanent or electromagnetic to draw out the magnetic beads and remove any unbound reporter beads in the suspension. Note that non-dissociated dual bead complexes not separated during Step VII will also be removed from the solution. During Step VIII in this method, the supernatant containing the released reporter beads 192 are collected using a pipette 214. The assay mixture may then be loaded into the disc and analyzed using an optical bio-disc reader, as illustrated in Step IX. Either a transmissive bio-disc 180 or a reflective bio-disc 144 may be used to analyze the reporter beads. Details relating to the reflective and transmissive optical bio-discs are discussed in detail with reference to FIGS. 3A-3C and 4A-4C, respectively. The optical bio-disc reader and other alternative disc readers that may be used to analyze optical bio-discs or medical CDs are described in detail above in conjunction with FIGS. 1 and 2. The reporter beads isolated in Step VIII may also be quantified using a fluorimeter or any similar fluorescent type analyzer including fluorescent optical disc readers.

As with any of the other methods discussed above, the magnetic removal or separation steps in the method shown in FIGS. 12B-1 and 12B-2 may be alternatively performed on-disc using the disc, fluidic circuits, and apparatus illustrated in FIGS. 33A-33D, 34A-34C, 35, and 36A-36C.

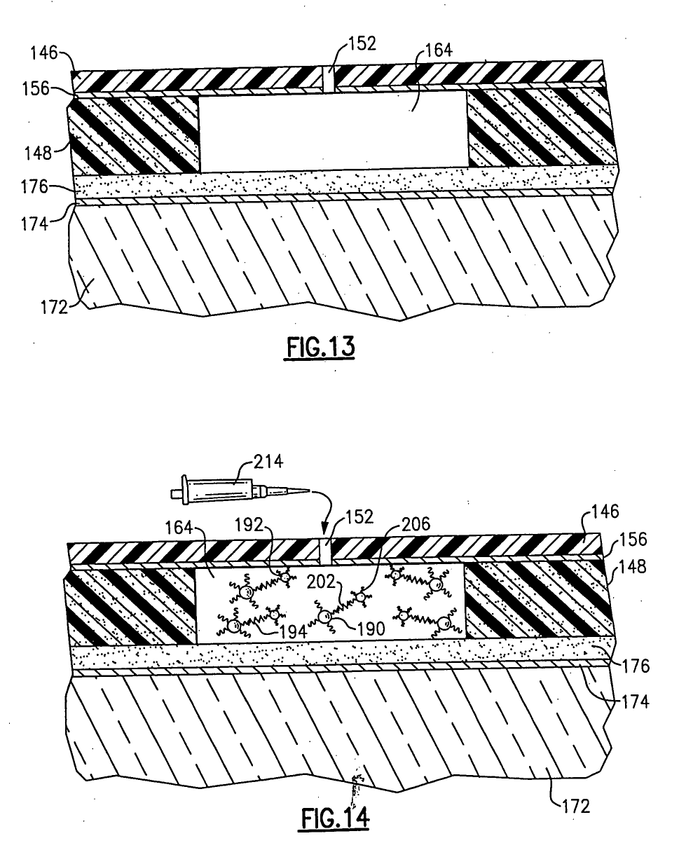

With reference now to FIG. 13, there is shown a cross sectional view illustrating the disc layers (similar to FIG. 6) of the mixing or loading chamber 164. Access to the loading chamber 164 is achieved by an inlet port 152 where the dual bead assay preparation is loaded into the disc system.

FIG. 14 is a view similar to FIG. 13 showing the mixing or loading chamber 164 with the pipette 214 injection of the dual bead complex 194 onto the disc. In this example, the complex includes reporters 192 and capture bead 190 linked together by the target DNA or RNA 202. The signal DNA 206 is illustrated as single stranded DNA complementary to the capture agent. The discs illustrated in FIGS. 13 and 14 may be readily adapted to other assays including the immunoassays and general molecular assays discussed above which employ, alternatively, proteins such as antigens or antibodies implemented as the transport probes, target agents, and signal probes accordingly.

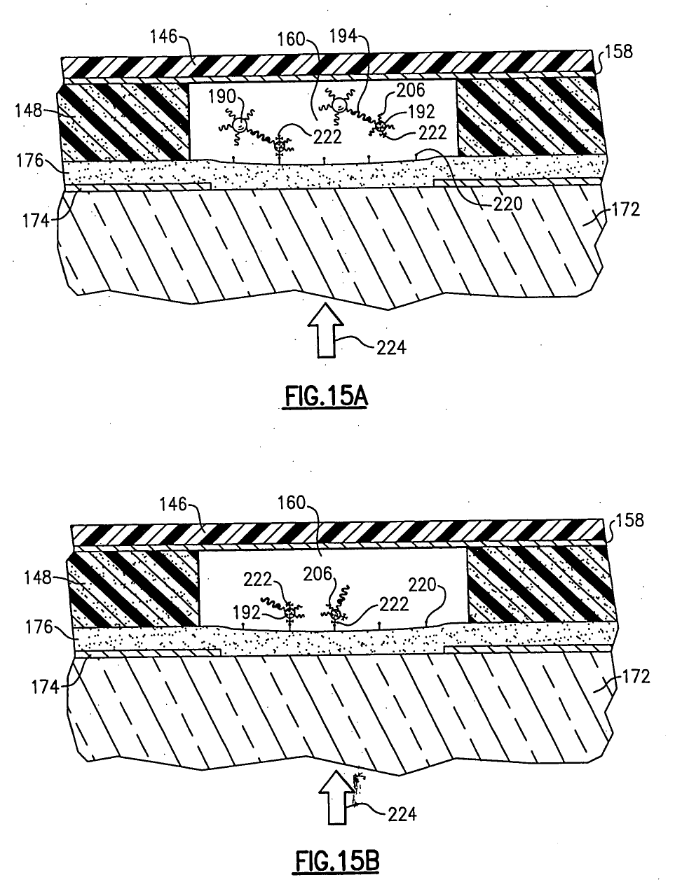

FIG. 15A shows the flow channel 160 and the target or capture zone 170 after anchoring of dual bead complex 194 to a capture agent 220. The capture agent 220 in this embodiment is attached to the active layer 176 by applying a small volume of capture agent solution to the active layer 176 to form clusters of capture agents within the area of the target zone 170. In this embodiment, the capture agent includes biotin or BSA-biotin. FIG. 15A also shows reporters 192 and capture beads 190 as components of a dual bead complex 194 as employed in the present invention. In this embodiment, anchor agents 222 are attached to the reporter beads 192. The anchor agent 222, in this embodiment, may include sterptavidin or Neutravidin. In this manner when the reporter beads 192 come in close proximity to the capture agents 220, binding occurs between the anchor probe 222/206 and the capture agent 220, via biotin-streptavidin interactions, thereby retaining the dual bead complex 194 within the target zone 170. At this point, an interrogation beam 224 directed to the target zone 170 can be used to detect the dual bead complex 194 within the target zone 170.

FIG. 15B is a cross sectional view similar to FIG. 15A illustrating the entrapment of the reporter bead 192 within the target zone 170 after a subsequent change in disc rotational speed. The change in rotational speed removes the capture beads 190 from the dual bead complex 194, ultimately isolating the reporter bead 192 in the target zone 170 to be detected by the interrogation or read beam 224.

The embodiment of the present invention discussed in conjunction with FIGS. 15A and 15B, may alternatively be implemented on the transmissive disc illustrated in FIGS. 4A-4C, 5B, and 6B.

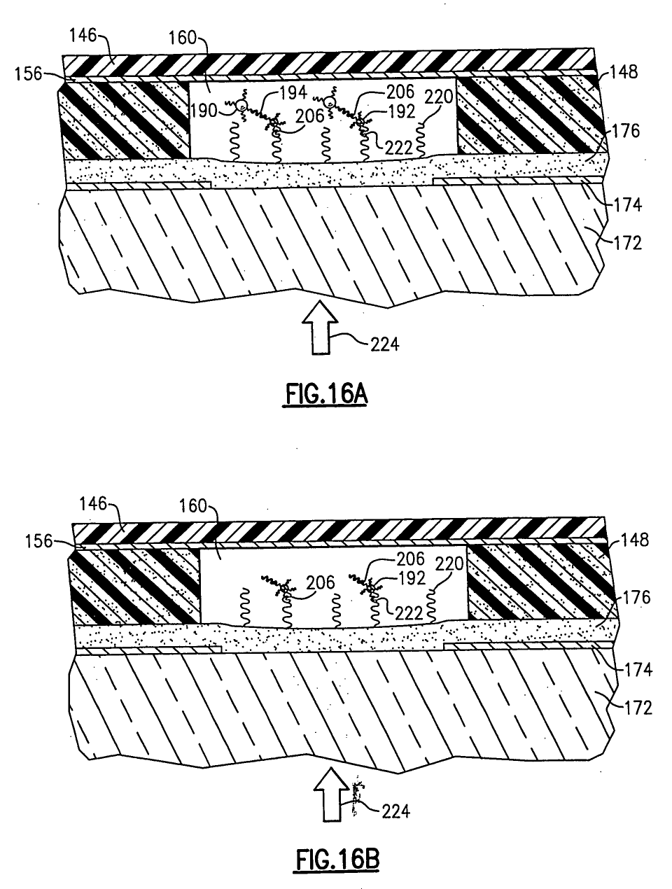

FIG. 16A is a cross sectional view, similar to FIG. 15A, that illustrates an alternative embodiment to FIG. 15A wherein the signal probes 206 or an anchor agent 222, on the reporter beads 192, directly hybridizes to the capture agent 220. FIG. 16A shows the flow channel 160 and the target or capture zone 170 after anchoring of dual bead complex 194 with the capture agent 220. The capture agent 220 in this embodiment is attached to the active layer 176 by applying a small volume of capture agent solution to the active layer 176 to form clusters of capture agents within the area of the target zone 170. Alternatively, the capture agent 220 may be attached to the active layer using an amino group that covalently binds to the active layer 176. In this embodiment, the capture agent includes DNA. FIG. 16A also shows reporters 192 and capture beads 190 as components of a dual bead complex 194 as employed in the present invention. In this embodiment, anchor probes 222 are attached to the reporter beads 192. The anchor agent 222, in this embodiment, may be a specific sequence of nucleic acids that are complimentary to the capture agent 220 or the oligonucleotide signal probe 206 itself. Thus when the reporter beads 192 come in close proximity to the capture agents 220, hybridization occurs between the anchor probe 222 and the capture agent 220 thereby retaining the dual bead complex 194 within the target zone 170. In an alternate embodiment, the signal probe 206 serves the function of anchor probe 222. At this point, an interrogation beam 224 directed to the target zone 170 may be used to detect the dual bead complex 194 within the target zone 170.

FIG. 16B illustrates the embodiment of FIG. 16A after a subsequent change in disc rotational speed. The change in rational speed removes the capture bead 190 from the dual bead complex 194, ultimately isolating the reporter bead 192 and the target DNA sequence 202 in the target zone 170 to be detected by an interrogation beam 224.

The embodiment of the present invention illustrated in FIGS. 16A and 16B, may alternatively be implemented on the transmissive disc shown in FIGS. 4A-4C, 5B, and 6B.

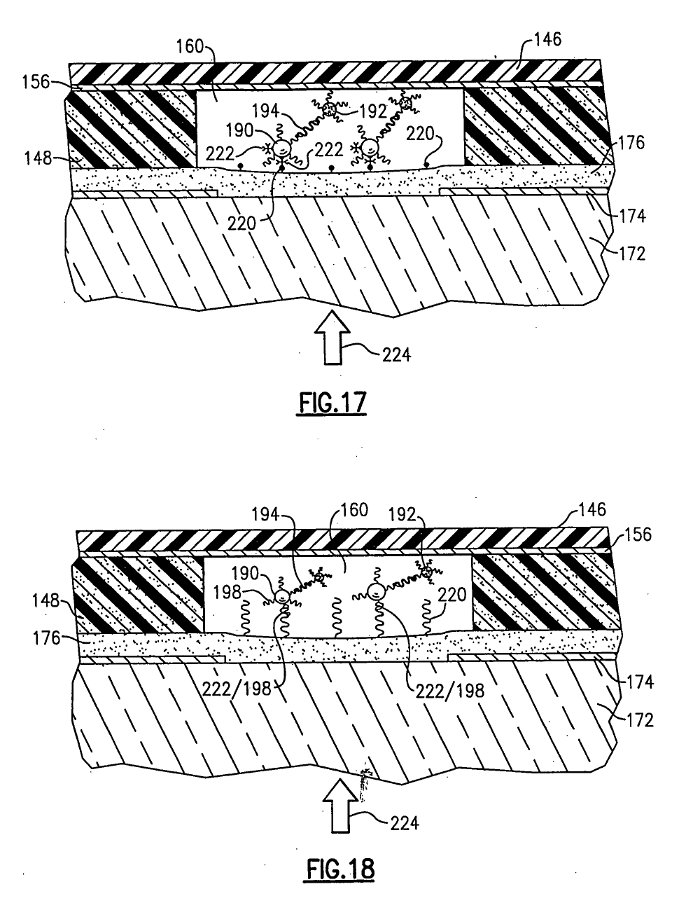

Referring now to FIG. 17, there is shown an alternative to the embodiment illustrated in FIG. 15A. In this embodiment, anchor agents 222 are attached to the capture beads 190 instead of the reporter beads. The anchor agent 222, in this embodiment, may include streptavidin or Neutravidin. As in FIG. 15A, the target zone 170 is coated with a capture agent 220. The capture agent may include biotin or BSA-biotin. FIG. 17 also shows reporters 192 and capture beads 190 as components of a dual bead complex 194 as employed in the present invention. When the capture beads 190 come in close proximity to the capture agents 220, binding occurs between the anchor probe 222 and the capture agent 220, via biotin-streptavidin interactions, thereby retaining the dual bead complex 194 within the target zone 170. At this point, an interrogation beam 224 directed to the target zone 170 can be used to detect the dual bead complex 194 within the target zone 170. The embodiment of the present invention shown in FIG. 17, may alternatively be implemented on the transmissive disc discussed in connection with in FIGS. 4A-4C, 5B, and 6B.

FIG. 18 is an alternative to the embodiment illustrated in FIG. 16A. In this embodiment, anchor agents 222 are attached to the capture beads 190 instead of the reporter beads. In this embodiment the transport probes 198, or an anchor agent 222 on the capture bead 190, directly hybridizes to the capture agent 220. In this embodiment, the capture agent 220 includes specific sequences of nucleic acid. The anchor agent 222, in this embodiment, may be a specific sequence of nucleic acids that are complimentary to the capture agent 220 or the oligonucleotide signal transport probe 198 itself, so that when the capture beads 190 come in close proximity to the capture agents 220, hybridization occurs between the anchor agent 222 and the capture agent 220. The dual bead complex 194 is thus thereby retained within the target zone 170. At this point, an interrogation beam 224 directed to the target zone 170 can be used to detect the dual bead complex 194 within the target zone 170. The embodiment of the present invention illustrated in FIG. 18, may alternatively be implemented on the transmissive disc shown in FIGS. 4A-4C, 5B, and 6B.

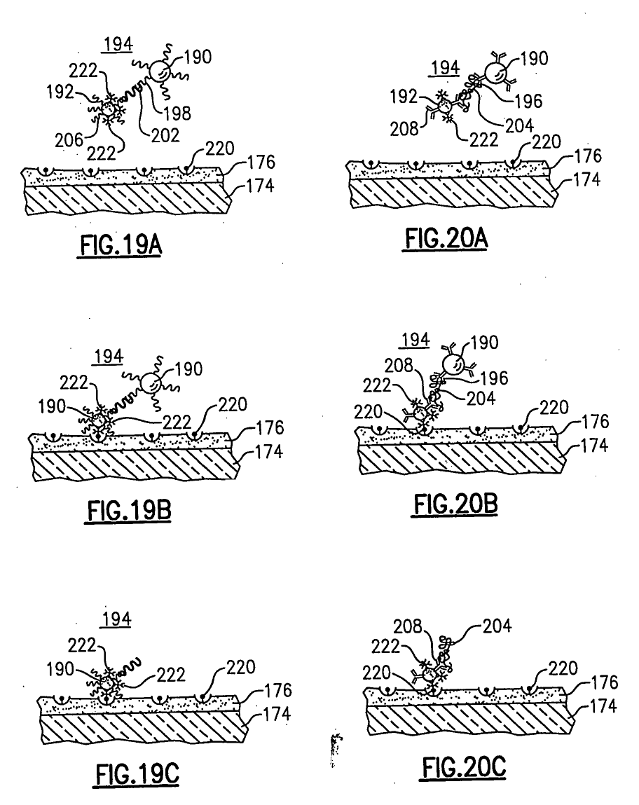

FIGS. 19A-19C are detailed partial cross sectional views showing the active layer 176 and the substrate 174 of the present bio-disc 110 as implemented in conjunction with the genetic assays discussed herein. FIGS. 19A-19C illustrate the capture agent 220 attached to the active layer 176 by applying a small volume of capture agent solution to the active layer 176 to form clusters of capture agents within the area of the target zone. The bond between capture agent 220 and the active layer 176 is sufficient so that the capture agent 220 remains attached to the active layer 176 within the target zone when the disc is rotated. FIGS. 19A and 19B also depict the capture bead 190 from the dual bead complex 194 binding to the capture agent 220 in the capture zone. These dual bead complexes are prepared according to the methods such as those discussed in FIGS. 11A-1, 11A-2, 12A-1 and 12A-2. The capture agent 220 includes biotin and BSA-biotin. In this embodiment, the reporter bead 192 anchors the dual bead complex 194 in the target zone via biotin/streptavidin interactions. Alternatively, the target zone may be coated with streptavidin and may bind biotinylated reporter beads. FIG. 19C illustrates an alternative embodiment which includes an additional step to those discussed in connection with FIGS. 19A and 19B. In this preferred embodiment, a variance in the disc rotations per minute may create a centrifugal force strong enough to break the capture beads 190 away from the dual bead complex 194 based on the differential size and/or mass of the bead. Although there is a shift in the rotation speed of the disc, the reporter bead 192 remains anchored to the target zone. Thus, the reporter beads 192 are maintained within the target zone and detected using an optical bio-disc or medical CD reader.

The embodiment of the present invention discussed in connection FIGS. 19A-19C, may be implemented on the reflective disc illustrated in FIGS. 3A-3C, 5A, and 6A or on the transmissive disc illustrated in FIGS. 4A-4C, 5B, and 6B.

FIGS. 20A, 20B, and 20C illustrate an alternative embodiment to the embodiment discussed in FIGS. 19A-19C. FIGS. 20A-20C show detailed partial cross sectional views of a target zone implemented in conjunction with immunochemical assays. FIGS. 20A and 20B also depict the capture bead 190 from the dual bead complex 194 binding to the capture agent 220 in the capture zone. The capture agent 220 includes biotin and BSA-biotin. These dual bead complexes may be prepared according to methods such as those discussed in FIGS. 11B-1, 11B-2, 12B-1 and 12B-2. In this embodiment, the reporter bead 192 anchors the dual bead complex 194 in the target zone via biotin/streptavidin interactions. The embodiment of the present invention illustrated in FIGS. 20A-20C, may be implemented on the reflective disc shown in FIGS. 3A-3C, 5A, and 6A or on the transmissive disc illustrated in FIGS. 4A-4C, 5B, and 6B.

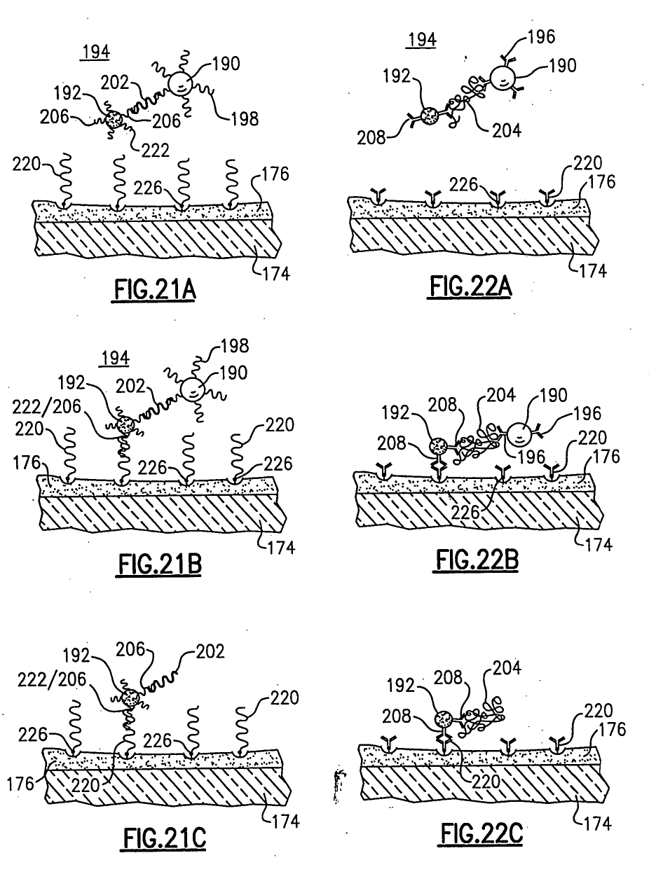

Referring now to FIGS. 21A, 21B, and 21C, there is shown detailed partial cross sectional views of a target zone including the active layer 176 and the substrate 174 of the present bio-disc 110 as implemented in conjunction with the genetic assays discussed herein. FIGS. 21A-21C illustrate the capture agent 220 attached to the active layer 176 by use of an amino group 226 which is made an integral part of the capture agent 220. As indicated, the capture agent 220 is situated within the target zone. The bond between the amino group 226 and the capture agent 220, and the amino group 226 and the active layer 176 is sufficient so that the capture agent 220 remains attached to the active layer 176 within the target zone when the disc is rotated. The preferred amino group 226 is NH₂. A thiol group may alternatively be employed in place of the amino group 226. In this embodiment of the present invention, the capture agent 220 includes the specific sequences of amino acids that are complimentary to the anchor agent 222 or oligonucleotide signal probe 206 which are attached to the reporter bead 192.

FIG. 21B depicts the reporter bead 192 of the dual bead complex 194, prepared according to methods such as those discussed in FIGS. 11A-1, 11A-2, 12A-1 and 12A-2, binding to the capture agent 220 in the target zone. As the dual bead complex 194 flows towards the capture agent 220 and is in sufficient proximity thereto, hybridization occurs between the anchor agent 222 or oligonucleotide signal probe 206 and the capture agent 220. Thus, the reporter bead 192 anchors the dual bead complex 192 within the target zone.

FIG. 21C illustrates an alternative embodiment that includes an additional step to those discussed in connection with FIGS. 21A-21B. In this preferred embodiment, a variance in the disc rotations per minute may create enough centrifugal force to break the capture beads 190 away from the dual bead complex 194 based on the differential size and/or mass of the bead. Although there is a shift in the rotation speed of the disc, the reporter bead 192 with the target DNA sequence 202 remains anchored to the target zone. In either case, the reporter beads 192 are maintained within the target zone as desired.

The embodiment of the present invention discussed with reference to FIGS. 21A-21C, may be implemented on the reflective disc shown in FIGS. 3A-3C, 5A, and 6A or on the transmissive disc illustrated in FIGS. 4A-4C, 5B, and 6B.

FIGS. 22A, 22B, and 22C illustrate an alternative embodiment to the embodiment discussed in FIGS. 21A-21C. FIGS. 22A-22C show detailed partial cross sectional views of a target zone implemented in conjunction with immunochemical assays. FIGS. 22A and 22B also depict the reporter bead 192 from the dual bead complex 194, (prepared according to methods such as those discussed in FIGS. 11B-1, 11B-2, 12B-1 and 12B-2), binding to the capture agent 220 in the capture zone. In this embodiment, the capture agent 220 includes antibodies bound to the target zone by use of an amino group 226 which is made an integral part of the capture agent 220. Alternatively, the capture agents 220 may be bound to the active layer 176 by passive absorption, and hydrophobic or ionic interactions. In this embodiment, the reporter bead 192 anchors the dual bead complex 194 in the target zone via specific antibody binding. As with the embodiment illustrated in FIG. 21C, FIG. 22C shows an alternative embodiment that includes an additional step to those discussed in connection with FIGS. 22A-22B. In this alternative embodiment, a variance in the disc's rotational speed may create enough centrifugal force to break the capture beads 190 away from the dual bead complex 194 based on the differential size and/or mass of the bead. Although there is a change in the rotational speed of the disc, the reporter bead 192 with the target antigen 204 remains anchored to the target zone. In either case, the reporter beads 192 are maintained within the target zone as desired. The embodiment of the present invention described in conjunction with FIGS. 22A-22C, may be implemented on the reflective disc illustrated in FIGS. 3A-3C, 5A, and 6A or on the transmissive disc shown in FIGS. 4A-4C, 5B, and 6B.

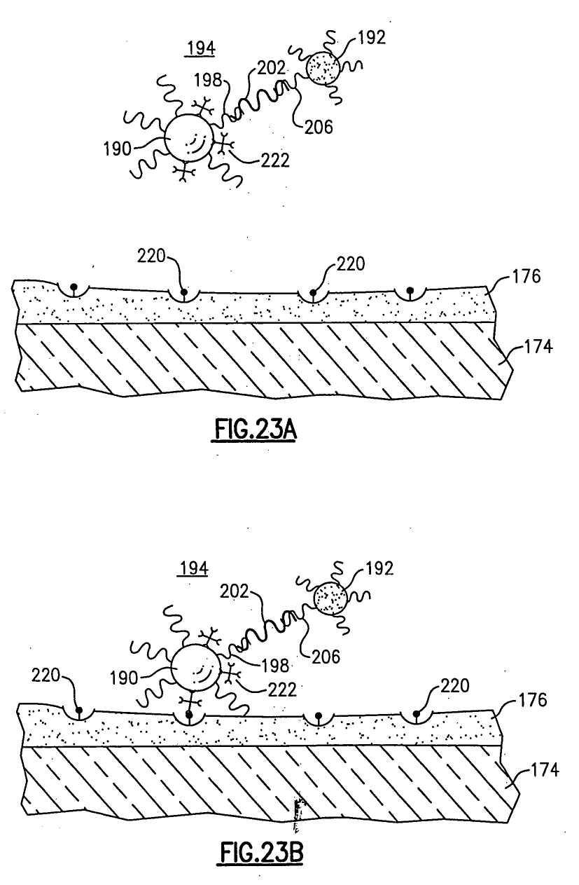

FIGS. 23A and 23B are detailed partial cross sectional views showing the active layer 176 and the substrate 174 of the present bio-disc 110 as implemented in conjunction with the genetic assays. FIGS. 23A and 23B illustrate an alternative embodiment to that discussed in FIGS. 19A and 19B above. In contrast to the embodiment in FIGS. 19A and 19B, in the present embodiment, the anchor agent 222 is attached to the capture bead 190 instead of the reporter bead 192. FIG. 23B illustrates the capture bead 190, from the dual bead complex 194, binding to the capture agent 220 in the capture zone. The capture agent 220 includes biotin and BSA-biotin. In this embodiment, the capture bead 190 anchors the dual bead complex 194 in the target zone via biotin/streptavidin interactions.

The embodiment of the present invention discussed with reference to FIGS. 23A and 23B, may be implemented on the reflective disc illustrated in FIGS. 3A-3C, 5A, and 6A or on the transmissive disc shown in FIGS. 4A-4C, 5B, and 6B.

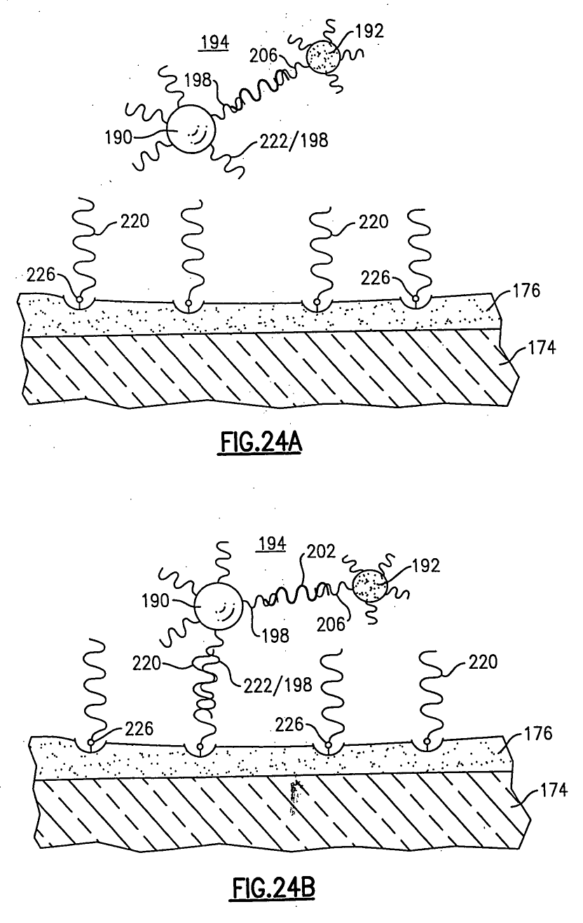

With reference now to FIGS. 24A and 24B, there is presented detailed partial cross sectional views showing the active layer 176 and the substrate 174 of the present bio-disc 110 as implemented in conjunction with the genetic assays. FIGS. 23A and 23B illustrate an alternative embodiment to that discussed in FIGS. 21A and 21B above. In contrast to the embodiment in FIGS. 21A and 21B, in the present embodiment, the anchor agent 222 is attached to the capture bead 190 instead of the reporter bead 192. FIG. 23B illustrates the capture bead 190 from the dual bead complex 194, binding to the capture agent 220 in the capture zone. The capture agent 220 is attached to the active layer 176 by use of an amino group 226 which is made an integral part of the capture agent 220. As indicated, the capture agent 220 is situated within the target zone. The bond between the amino group 226 and the capture agent 220, and the amino group 226 and the active layer 176 is sufficient so that the capture agent 220 remains attached to the active layer 176 within the target zone when the disc is rotated. In this embodiment of the present invention, the capture agent 220 includes the specific sequences of amino acids that are complimentary to the anchor agent 222 or oligonucleotide transport probe 198 which are attached to the capture bead 190. Also in this embodiment, the capture bead 190 anchors the dual bead complex 194 in the target zone via hybridization between the capture agent 220 and the anchor agent or the transport probe 198.

The embodiment of the present invention shown in FIGS. 24A and 24B, may be implemented on the reflective disc illustrated in FIGS. 3A-3C, 5A, and 6A or on the transmissive disc depicted in FIGS. 4A-4C, 5B, and 6B.

Disc Processing Methods

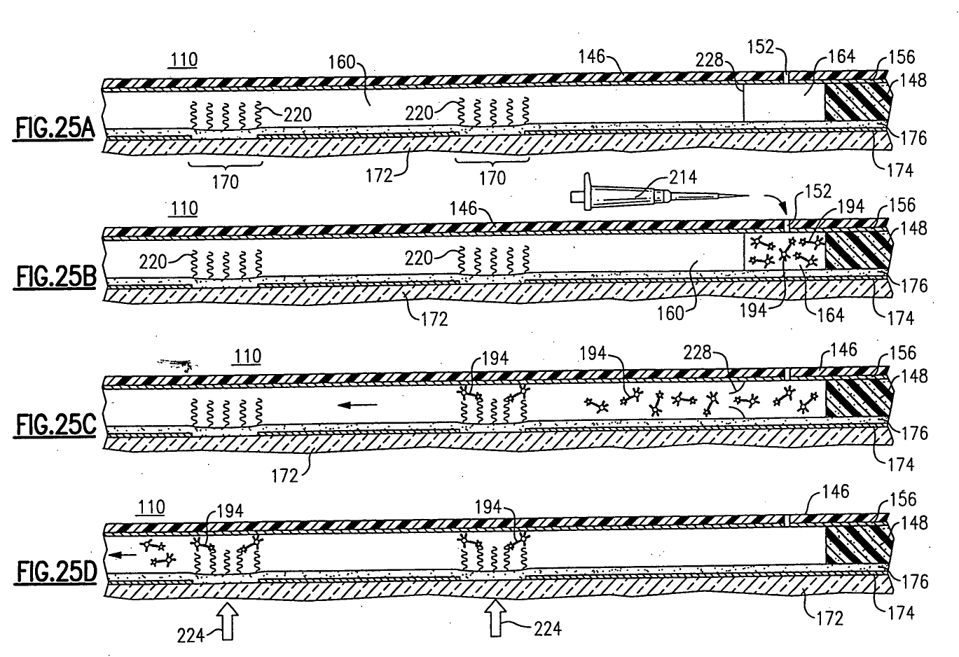

Turning now to FIGS. 25A-25D, there is shown the target zones 170 set out in FIGS. 21A-21C and FIGS. 24A-24B in the context of a disc, using as an input the solution created according to methods such as those shown in FIGS. 11A-1, 11A-2, 12A-1 and 12A-2.

FIG. 25A shows a mixing/loading chamber 164, accessible through an inlet port 152, leading to a flow channel 160. Flow channel 160 is pre-loaded with capture agents 220 situated in clusters. Each of the clusters of capture agents 220 is situated within a respective target zone 170. Each target zone 170 can have one type of capture agent or multiple types of capture agents, and separate target zones can have one and the same type of capture agent or multiple different capture agents in multiple capture fields. In the present embodiment, the capture agent can include specific sequences of nucleic acids that are complimentary to anchor agents 222 on either the reporter 192 or capture bead 190.

In FIG. 25B, a pipette 214 is loaded with a test sample of DNA or RNA that has been sequestered in the dual bead complex 194. The dual bead complex is injected into the flow channel 160 through inlet port 152. As flow channel 160 is further filled with the dual bead complex from pipette 214, the dual bead complex 194 begins to move down flow channel 160 as the disc is rotated. The loading chamber 164 can include a break-away retaining wall 228 so that complex 194 moves down the flow channel at one time.

In this embodiment, anchor agents 222, attached to reporter beads 192, bind to the capture agents 220 by hybridization, as illustrated in FIG. 25C. In this manner, reporter beads 192 are retained within target zone 170. Binding can be further facilitated by rotating the disc so that the dual bead complex 194 can slowly move or tumble down the flow channel. Slow movement allows ample time for additional hybridization. After hybridization, the disc can be rotated further at the same speed or faster to clear target zone 170 of any unattached dual bead complex 194, as illustrated in FIG. 25D.

An interrogation beam 224 can then be directed through target zones 170 to determine the presence of reporters, capture beads, and dual bead complex, as illustrated in FIG. 25D. In the event no target DNA or RNA is present in the test sample, there will be no dual bead complex structures, reporters, or capture beads bound to the target zones 170, but a small amount of background signal may be detected in the target zones from non-specific binding. In this case, when the interrogation beam 224 is directed into the target zone 170, a zero or low reading results, thereby indicating that no target DNA or RNA was present in the sample.

The speed, direction, and stages of rotation, such as one speed for one period followed by another speed for another period, can all be encoded in the operational information on the disc. The method discussed in connection with FIGS. 25A-25D may also be performed on the transmissive disc illustrated in FIGS. 4A-4C, 5B, and 6B using a system with the top detector 130.

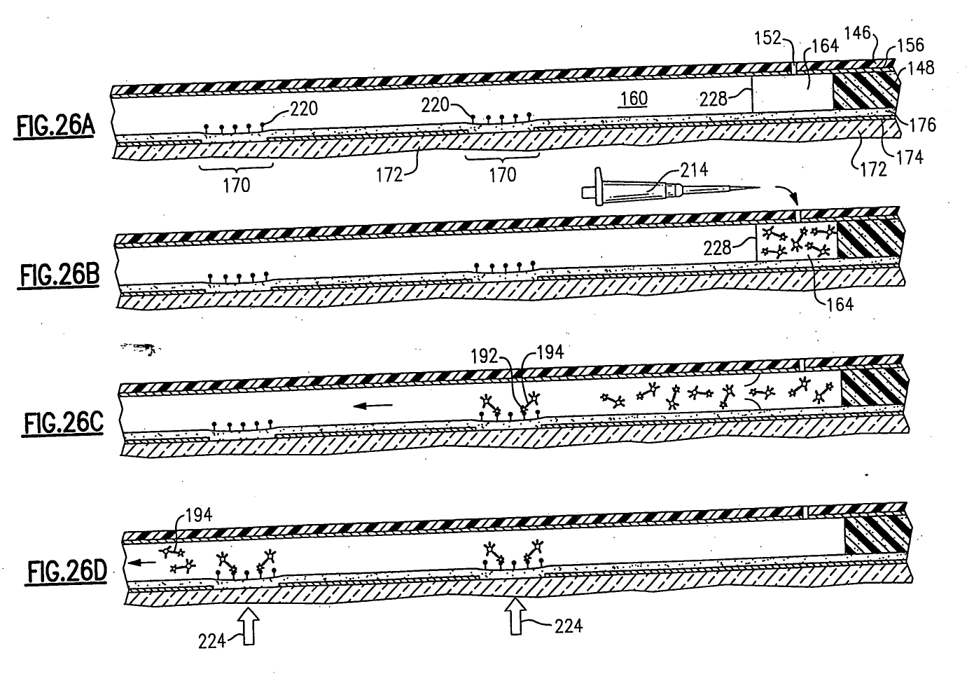

FIGS. 26A-26D show the target zones 170 including the capture chemistries discussed in FIGS. 19A-19C and FIGS. 23A-23B. This method uses as an input, the solution created according to methods shown in FIGS. 1A-1, 11A-2, 12A-1 and 12A-2. FIGS. 26A-26D illustrate an alternative embodiment to that discussed in FIGS. 25A-25D showing a different bead capture method described in further detail below.

FIG. 26A shows a mixing/loading chamber 164, accessible through an inlet port 152, leading to a flow channel 160. Flow channel 160 is pre-loaded with capture agents 220 situated in clusters. Each of the clusters of capture agents 220 is situated within a respective target zone 170. Each target zone 170 can have one type of capture agent or multiple types of capture agents, and separate target zones can have one and the same type of capture agent or multiple different capture agents in multiple capture fields. In the present embodiment, the capture agent can include specific biotin and BSA-biotin that has affinity to the anchor agents 222 on either the reporter 192 or capture bead 190. The anchor agents may include streptavidin and Neutravidin.

In FIG. 26B, a pipette 214 is loaded with a test sample of DNA or RNA that has been sequestered in the dual bead complex 194. The dual bead complex is injected into the flow channel 160 through inlet port 152. As flow channel 160 is further filled with the dual bead complex from pipette 214, the dual bead complex 194 begins to move down flow channel 160 as the disc is rotated. The loading chamber 164 can include a break-away retaining wall 228 so that complex 194 moves down the flow channel at one time.

In this embodiment, anchor agents 222, attached to reporter beads 192, bind to the capture agents 220 by biotin-streptavidin interactions, as illustrated in FIG. 26C. In this manner, reporter beads 192 are retained within target zone 170. Binding can be further facilitated by rotating the disc so that the dual bead complex 194 can slowly move or tumble down the flow channel. Slow movement allows ample time for additional binding between the capture agent 220 and the anchor agent 222. After binding, the disc can be rotated further at the same speed or faster to clear target zone 170 of any unattached dual bead complex 194, as illustrated in FIG. 26D.

An interrogation beam 224 can then be directed through target zones 170 to determine the presence of reporters, capture beads, and dual bead complex, as illustrated in FIG. 26D. In the event no target DNA is present in the test sample, there will be no dual bead complex structures beads bound to the target zones 170. A small amount of background signal may be detected in the target zones from non-specific binding. In this case, when the interrogation beam 224 is directed into the target zone 170, a zero or low reading results, thereby indicating that no target DNA or RNA was present in the sample.

The speed, direction, and stages of rotation, such as one speed for one period followed by another speed for another period, can all be encoded in the operational information on the disc.

The method discussed in conjunction with FIGS. 26A-26D was illustrated on a reflective disc such as the disc shown in FIGS. 3A-3C, 5A, and 6A. This method may also be performed on the transmissive disc shown in FIGS. 4A-4C, 5B, and 6B using a system with the top detector 130.

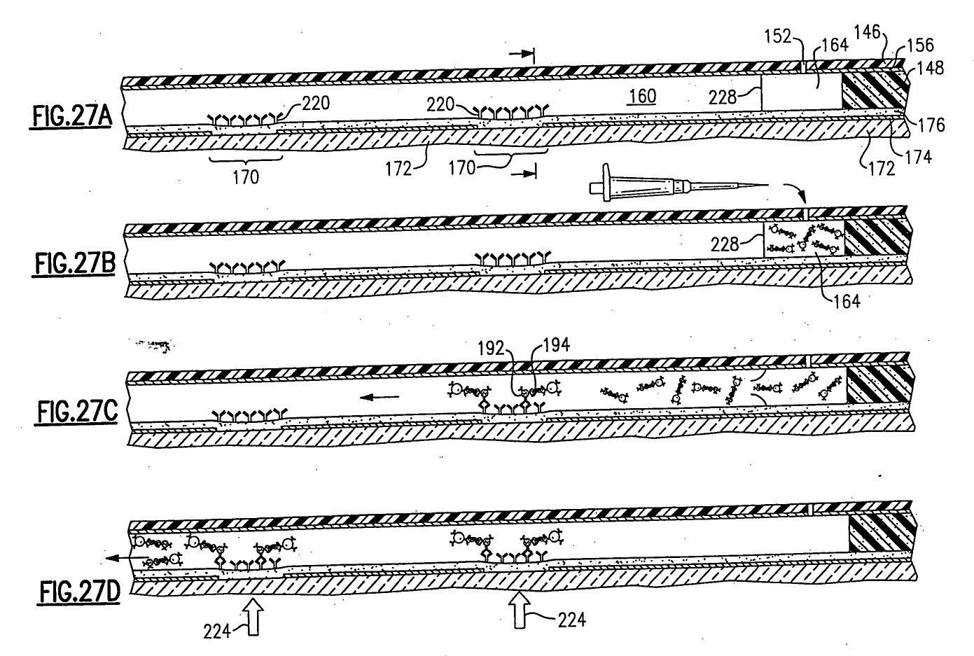

Referring next to FIGS. 27A-27D there is shown a series of cross sectional side views illustrating the steps of yet another alternative method according to the present invention. FIGS. 27A-27D show the target zones 170 including the capture mechanisms discussed in connection with FIGS. 22A-22C. This method uses an input the solution created according to the preparation methods shown in FIGS. 11B-1, 11B-2, 12B-1 and 12B-2. FIGS. 27A-27D illustrate an immunochemical assay and an alternative bead capture method.

FIG. 27A shows a mixing/loading chamber 164, accessible through an inlet port 152, leading to a flow channel 160. Flow channel 160 is pre-loaded with capture agents 220 situated in clusters. Each of the clusters of capture agents 220 is situated within a respective target zone 170. Each target zone 170 can have one type of capture agent or multiple types of capture agents, and separate target zones can have one and the same type of capture agent or multiple different capture agents in multiple capture fields. In the present embodiment, the capture agent can include antibodies that specifically bind to epitopes on the anchor agents 222 on either the reporter 192 or capture bead 190. Alternatively, the capture agent can directly bind to epitopes on the target antigen 204 within the dual bead complex 194. The anchor agents can include the target antigen, antibody transport probe 196, the antibody signal probe 208, or any antigen, bound to either the reporter bead 192 or the capture bead 190, that has epitopes than can specifically bind to the capture agent 220.

In FIG. 27B, a pipette 214 is loaded with a test sample of target antigen that has been sequestered in the dual bead complex 194. The dual bead complex is injected into the flow channel 160 through inlet port 152. As flow channel 160 is further filled with the dual bead complex from pipette 214, the dual bead complex 194 begins to move down flow channel 160 as the disc is rotated. The loading chamber 164 may include a break-away retaining wall 228 so that complex 194 moves down the flow channel at one time.

In this embodiment, anchor agents 222, attached to reporter beads 192, bind to the capture agents 220 by antibody-antigen interactions, as illustrated in FIG. 27C. In this manner, reporter beads 192 are retained within target zone 170. Binding can be further facilitated by rotating the disc so that the dual bead complex 194 can slowly move or tumble down the flow channel. Slow movement allows ample time for additional binding between the capture agents 220 and the anchor agent 222. After binding, the disc can be rotated further at the same speed or faster to clear target zone 170 of any unattached dual bead complex 194, as illustrated in FIG. 27D.

An interrogation beam 224 can then be directed through target zones 170 to determine the presence of reporters, capture beads, and dual bead complex, as illustrated in FIG. 27D. In the event no target antigen is present in the test sample, there will be no dual bead complex structures, reporters, or capture beads bound to the target zones 170, but a small amount of background signal may be detected in the target zones from non-specific binding. In this case, when the interrogation beam 224 is directed into the target zone 170, a zero or low reading results, thereby indicating that no target was present in the sample.

The speed, direction, and stages of rotation, such as one speed for one period followed by another speed for another period, can all be encoded in the operational information on the disc.

The methods described in FIGS. 25A-25D, 26A-26D, and 27A-27D are implemented using the reflective disc system 144. As indicated above, it should be understood that these methods and any other bead or sphere detection may also be carried out using the transmissive disc embodiment 180, as described in FIGS. 4A-4C, 5B, and 6B. It should also be understood that the methods described in FIGS. 11A-1, 11A-2, 11B-1, 11B-2, 12A-1, 12A-2, 12B-1, 12B-2, 25A-25D, 26A-26D, and 27A-27D are not limited to creating the dual bead complexes outside of the optical bio-discs but may include embodiments that use “in-disc” or “on-disc” formation of the dual bead complexes. In these on-disc implementations the dual bead complex is formed within the fluidic circuits of the optical bio-disc 110. For example, the dual bead formation may be carried out in the loading or mixing chamber 164. In one embodiment, the beads and sample are added to the disc at the same time, or nearly the same time. Alternatively, the beads with the probes can be pre-loaded on the disc for future use with a sample so that only a sample needs to be added.

The beads would typically have a long shelf life, with less shelf life for the probes. The probes can be dried or lyophilized (freeze dried) to extend the period during which the probes can remain in the disc. With the probes dried, the sample essentially reconstitutes the probes and then mixes with the beads to produce dual bead complex structures can be performed.

In either case, the basic process for on disc processing includes: (1) inserting the sample into a disc with beads with probes; (2) causing the sample and the beads to mix on the disc; (3) isolating, such as by applying a magnetic field, to hold the dual bead complex and move the non-held beads away, such as to a region referred to here as a waste chamber; and (4) directing the dual bead complexes (and any other material not moved to the waste chamber) to the capture fields. The detection process can be the same as one of those described above, such as by event detection or fluorimetry.

In addition to the above, it would be apparent to those of skill in the art that the disc surface capturing techniques and the linking techniques for forming the dual bead complexes illustrated in FIGS. 25A-25D, 26A-26D, and 27A-27D may be interchanged to create alternate variations thereof. For example, the inventors have contemplated that the capture agents 220 as implemented to include specific sequences of nucleic acids may be used to capture dual bead complexes formed by either DNA hybridization as illustrated in FIG. 10A or the antibody-antigen interactions shown in FIG. 10B. Similarly, capture agents 220 as implemented to include antibodies may be employed to capture dual bead complexes formed by either the DNA hybridization method shown in FIG. 10A or the antibody-antigen interactions illustrated in FIG. 10B. And also, capture agents 220 as implemented to include biotin or BSA-biotin may be similarly utilized to capture dual bead complexes formed by either the DNA hybridization techniques illustrated in FIG. 10A or the antibody-antigen interactions depicted in FIG. 10B. Other combinations including different anchor agents to perform the binding function with the capture agent, are readily apparent from the present disclosure and are thus specifically provided for herein.

Detection and Related Signal Processing Methods and Apparatus

The number of reporter beads bound in the capture field can be detected in a qualitative manner, and may also be quantified by the optical disc reader.

The test results of any of the test methods described above can be readily displayed on monitor 114 as illustrated in FIG. 1. The disc according to the present invention preferably includes encoded software that is read to control the controller, the processor, and the analyzer as shown in FIG. 2. This interactive software is implemented to facilitate the methods described herein and the display of results.

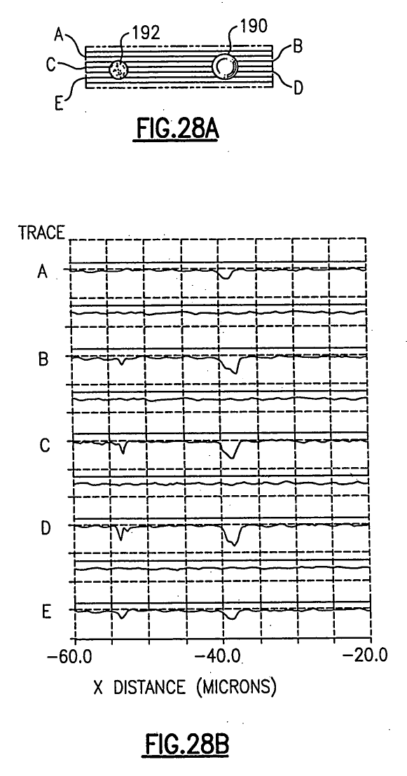

FIG. 28A is a graphical representation of an individual 2.1 micron reporter bead 192 and a 3 micron capture bead 190 positioned relative to tracks A, B, C, D, and E of an optical bio-disc or medical CD according to the present invention.

FIG. 28B is a series of signature traces, from tracks A, B, C, D, and E, derived from the beads of FIG. 28A utilizing a detected signal from the optical drive according to the present invention. These graphs represent the detected return beam 124 of the reflective disc illustrated in FIGS. 5A and 6A for example, or the transmitted beam 128 of the transmissive disc illustrated in FIGS. 5B and 6B. As shown, the signatures for a 2.1 micron reporter bead 190 are sufficiently different from those for a 3 micron capture bead 192 such that the two different types of beads can be detected and discriminated. A sufficient deflection of the trace signal from the detected return beam as it passes through a bead is referred to as an event.

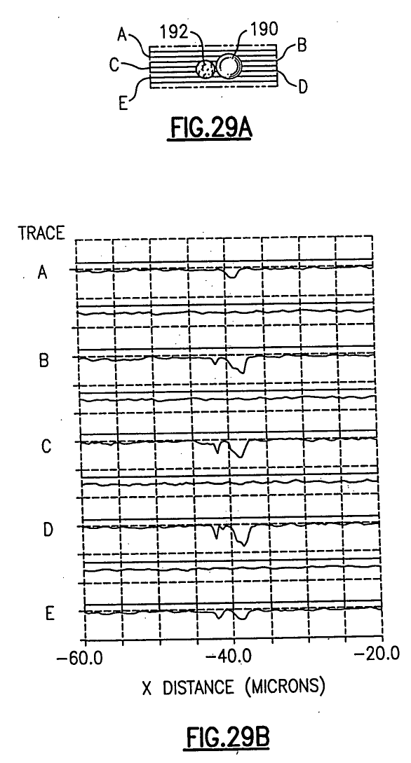

FIG. 29A is a graphical representation of a 2.1 micron reporter bead and a 3 micron capture bead linked together in a dual bead complex positioned relative to the tracks A, B, C, D, and E of an optical bio-disc or medical CD according to the present invention.

FIG. 29B is a series of signature traces, from tracks A, B, C, D, and E, derived from the beads of FIG. 29A utilizing a detected signal from the optical drive according to the present invention. These graphs represent the detected return beam 124 of a reflective disc 144 or transmitted beam 128 of a transmissive disc 180. As shown, the signatures for a 2.1 micron reporter bead 190 are sufficiently different from those for a 3 micron capture bead 192 such that the two different types of beads can be detected and discriminated. A sufficient deflection of the trace signal from the detected return or transmitted beam as it passes through a bead is referred to as an event. The relative proximity of the events from the reporter and capture bead indicates the presence or absence the dual bead complex. As shown, the traces for the reporter and the capture bead are right next to each other indicating the beads are joined in a dual bead complex.

Alternatively, other detection methods can be used. For example, reporter beads can be fluorescent or phosphorescent. Detection of these reporters can be carried out in fluorescent or phosphorescent type optical disc readers. Other signal detection methods are described, for example, in commonly assigned co-pending U.S. patent application Ser. No. 10/008,156 entitled “Disc Drive System and Methods for Use with Bio-Discs” filed Nov. 9, 2001, which is expressly incorporated by reference; U.S. Provisional Application Ser. Nos. 60/270,095 filed Feb. 20, 2001 and 60/292,108, filed May 18, 2001; and the above referenced U.S. patent application Ser. No. 10/043,688 entitled “Optical Disc Analysis System Including Related Methods For Biological and Medical Imaging” filed Jan. 10, 2002.

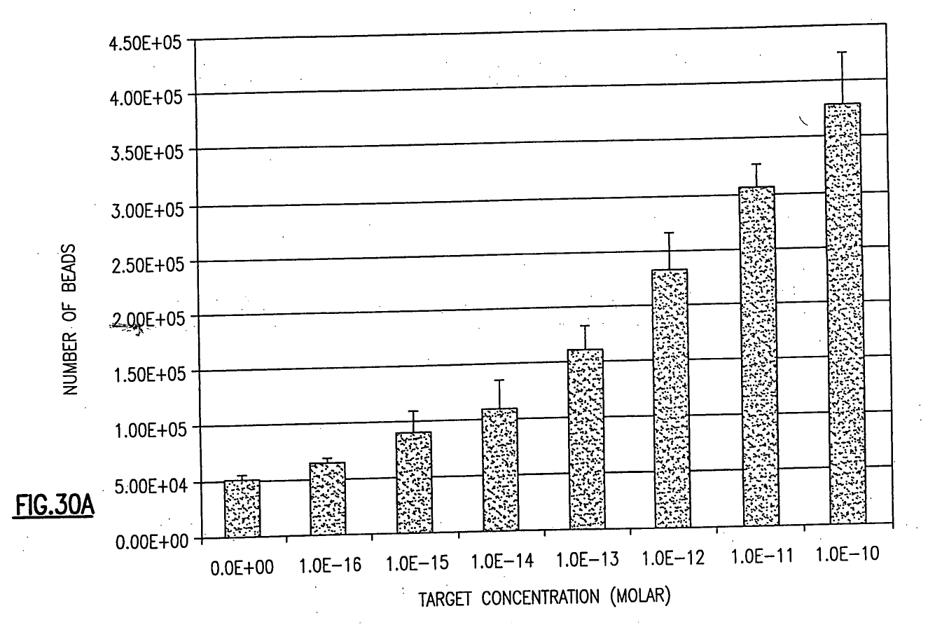

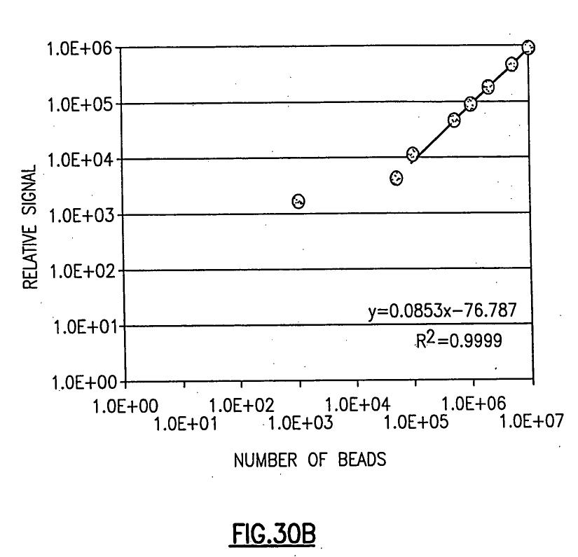

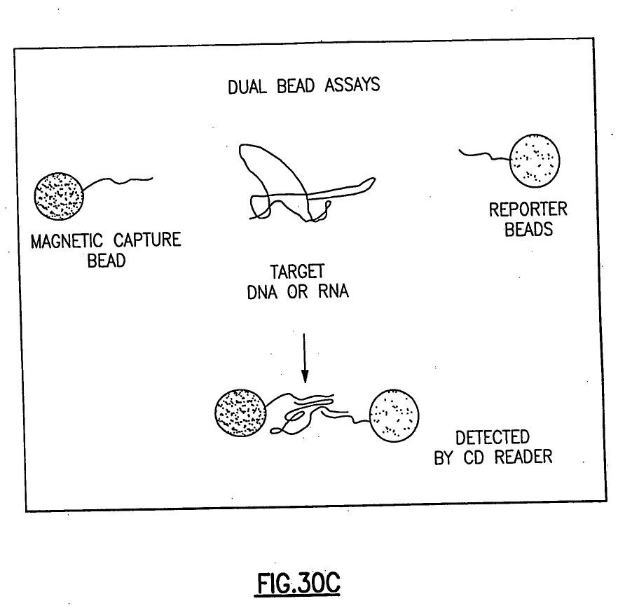

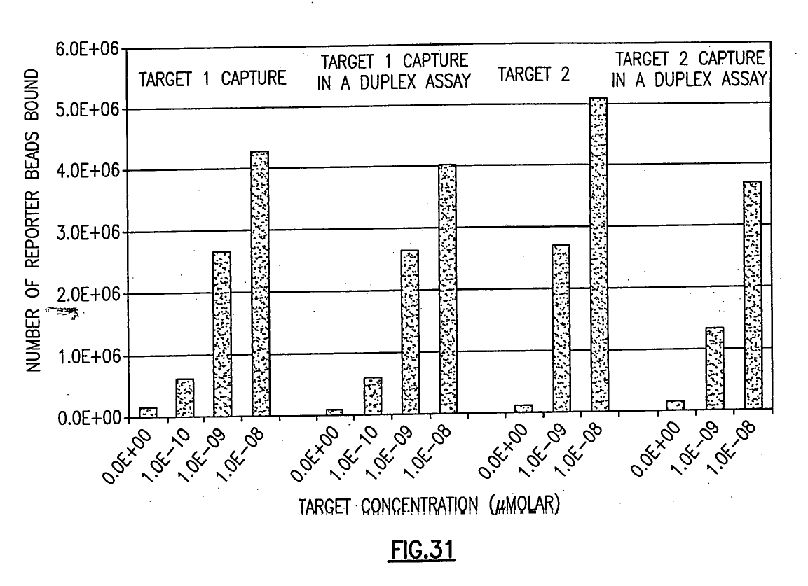

FIG. 30A is a bar graph of data generated using a fluorimeter showing a concentration dependent target detection using fluorescent reporter beads. This graph shows the molar concentration of target DNA versus number of detected beads. The dynamic range of target detection shown in the graph is 10E-16 to 10E-10 Molar (moles/liter). While the particular graph shown was generated using data from a fluorimeter, the results may also be generated using a fluorescent type optical disc drive.

FIG. 30B presents a standard curve demonstrating that the sensitivity of a fluorimeter is approximately 1000 beads in a fluorescent dual bead assay. The sensitivity of any assay depends on the sensitivity of the assay itself and on the sensitivity of the detection system. Referring to FIGS. 30A-30C, various studies were done to examine the sensitivity of the dual bead assay using different detection methods, e.g., a fluorimeter, and bio-disc or medical CD detection according to the present invention.

As shown in FIG. 30B, the sensitivity of a fluorimeter is approximately 1000 beads in a fluorescent dual bead assay. In contrast, FIG. 30A shows that even at 10E-16 Molar (moles/liter), a sufficient number of beads over zero concentration can be detected to sense the presence of the target. With a sensitivity of 10E-16 Molar, a dual bead assay represents a very sensitive detection method for DNA that does not require DNA amplification (such as through PCR) and can be used to detect even a single bead.

In contrast to conventional detection methods, the use of a medical CD or bio-disc coupled with a CD-reader or optical bio-disc drive (FIG. 1) improves the sensitivity of detection. For example, while detection with a fluorimeter is limited to approximately 1000 beads (FIG. 30B), use of a bio-disc coupled with CD-reader may enable the user to detect a single bead with the interrogation beam as illustrated in FIGS. 29A, 29B, and 30C. Thus, the bioassay system provided herein improves the sensitivity of dual bead assays significantly.

The detection of single beads using an optical bio-disc or medical CD is discussed in detail in conjunction with FIGS. 28A and 28B. FIG. 28B shows the signal traces of each bead as detected by the medical CD or bio-disc reader. Dual bead complexes may also be identified by the bio-disc reader using the unique signature trace collected from the detection of a dual bead complex as shown in FIGS. 29A and 29B. Different optical bio-disc platforms may be used in conjunction with the reader device for detection of beads including a reflective and a transmissive disc format illustrated in FIGS. 3C and 4C, respectively.

FIG. 30C is a pictorial demonstrating the formation of the dual bead complex linked together by the presence of the target in a genetic assay. Sensitivity to within one reporter molecule is possible with the present dual bead assay quantified with a bio-CD reader shown in FIGS. 1 and 2 above. Similarly, the dual bead complex formation may also be implemented in an immunochemical assay format as illustrated above in FIGS. 7B, 8B, 9B, 10B, 11B-1, 11B-2, 12B-1, and 12B-2.

FIG. 31 shows data generated using a fluorimeter illustrating the concentration dependent detection of two different targets. The target detection was carried out in two different methods, the single and the duplex assays. In the single assay, the capture bead contains a transport probe specific to a single target and a reporter probe coated with a signal probe specific to the same target is mixed in a solution together with the target. In the duplex assay, the capture bead contains two different transport probes specific to two different targets. Experimental details regarding the use of the duplex target detection method are discussed in further detail in Example 2. Mixing different reporter beads (red and green fluorescent or silica and polystyrene beads, for example) containing signal probes specific to one of the two targets, allows the detection of two different targets simultaneously.

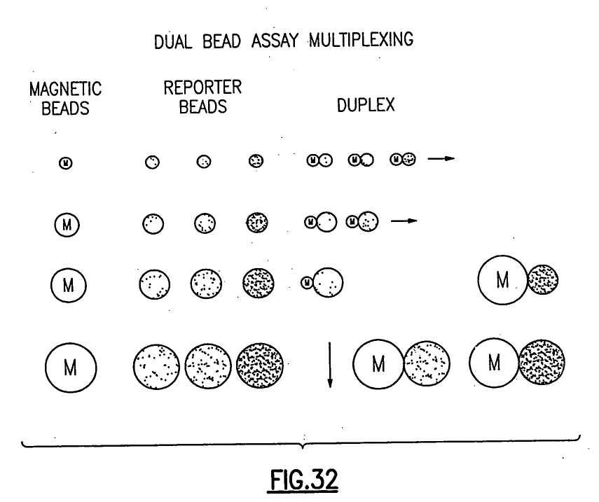

Detection of the dual bead duplex assay may be carried out using a magneto optical disc system described below. FIGS. 32 and 37 illustrate the formation and binding of various dual bead complexes onto an optical disc which may be detected by an optical bio-disc drive (FIG. 2), a magneto-optical disc system, a fluorescent disc system, or any similar device. Unique signature traces of a dual bead complex collected from an optical disc reader are shown in FIG. 29B above. The traces from FIG. 29B further illustrate that different bead types can be detected by an optical disc reader since different type beads will show a different signature profile.

Multiplexing, Magneto-Optical, and Magnetic Discs Systems

The use of a dual bead assay in the capture of targets allows for the use in multiplexing assays. This type of multiplexing is achieved by combining different sizes of magnetic beads with different types and sizes of reporter beads. Thus, different target agents can be detected simultaneously. As indicated in FIG. 32, four sizes of magnetic capture beads, and four sizes of three types of reporter beads produce up to 48 different types of dual bead complex. In a multiplexing assay, probes specific to different targets are thus conjugated to capture beads. Reporter beads having different physical and/or optical properties, such as fluorescence at different wavelengths allow for simultaneous the detection of different target agents from the same biological sample. As indicated in FIGS. 28A, 28B, 29A, and 29B, small differences in size can be detected by detecting reflected or transmitted light.

Multiple dual bead complex structures for capturing different target agents can be carried out on or off the disc. The dual bead suspension is loaded into a port on the disc. The port is sealed and the disc is rotated in the disc reader. During spinning, free (unbound) beads are spun off to a periphery of the disc. The reporter beads detecting various target agents are thus localized in capture fields. In this manner, the presence of a specific target agent can be detected, and the amount of a specific target agent can be quantified by the disc reader.

FIG. 33A is a general representation of an optical disc according to another aspect of the present invention and a method corresponding generally to the single-step method of FIGS. 11A-1, 11A-2, 11B-2 and 11B-2. The sample and beads can be added at one time or successively but closely in time. Alternatively, the beads can be pre-loaded into a portion of the disc. These materials can be provided to a mixing chamber 164 that can have a breakaway wall 228 (see FIG. 25A), which holds in the solution within the mixing chamber 164. Mixing the sample and beads on the disc would be accomplished through rotation at a rate insufficient to cause the wall to break or the capillary forces to be overcome.

The disc can be rotated in one direction, or it can be rotated alternately in opposite directions to agitate the material in a mixing chamber. The mixing chamber is preferably sufficiently large so that circulation and mixing is possible. The mixing can be continuous or intermittent.

FIG. 33B shows one embodiment of a rotationally directionally dependent valve arrangement that is directionally dependent and uses a movable component for a valve. The mixing chamber leads to an intermediate chamber 244 that has a movable component, such as a ball 246. In the non-rotated state, the ball 246 may be kept in a slight recessed portion, or chamber 244 may have a gradual V-shaped tapering in the circumferential direction to keep the ball centered when there is no rotation.

Referring to FIGS. 33C and 33D in addition to FIGS. 33A and 33B, when the disc is rotated clockwise (FIG. 33C), ball 246 moves to a first valve seat 248 to block passage to detection chamber 234 and to allow flow to waste chamber 232, shown in FIG. 33A. When the disc is rotated counter-clockwise (FIG. 33D), ball 246 moves to a second valve seat 250 to block a passage to waste chamber 232 and to allow flow to detection chamber 234.

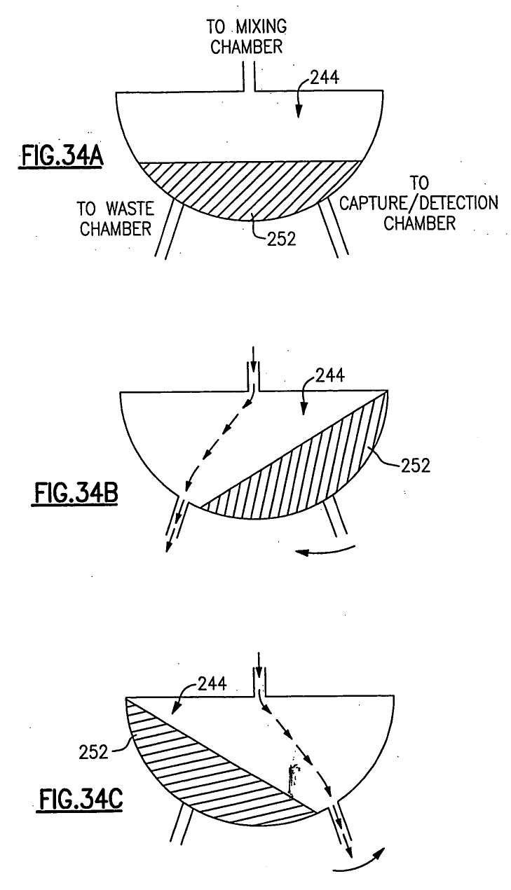

FIGS. 34A-34C show a variation of the prior embodiment in which the ball is replaced by a wedge 252 that moves one way or the other in response to acceleration of the disc. The wedge 252 can have a circular outer shape that conforms to the shape of an intermediate chamber 244. The wedge is preferably made of a heavy dense material relative to chamber 244 to avoid sticking. A coating can be used to promote sliding of the wedge relative to the chamber.

When the disc is initially rotated clockwise as shown in FIG. 34B, the angular acceleration causes wedge 252 to move to block a passage to detection chamber 234 and to allow flow to waste chamber 232. When the disc is initially rotated counter-clockwise, FIG. 34C, the angular acceleration causes wedge 252 moves to block a passage to waste chamber 232 and allow flow to detection chamber 234. During constant rotation after the acceleration, wedge 252 remains in place blocking the appropriate passage.

In another embodiment of the present invention where the capture beads are magnetic, a magnetic field from a magnetic field generator or field coil 230 can be applied over the mixing chamber 164 to hold the dual bead complexes and unbound magnetic beads in place while material without magnetic beads is allowed to flow away to a waste chamber 232. This technique may also be employed to aid in mixing of the assay solution within the fluidic circuits or channels before any unwanted material is washed away. At this stage, only magnetic capture beads, unbound or as part of a dual bead complex, remain. The magnetic field is released, and the dual bead complex with the magnetic beads is directed to a capture and detection chamber 234.

The process of directing non-magnetic beads to waste chamber 232 and then magnetic beads to capture chamber 234 can be accomplished through the microfluidic construction and/or fluidic components. A flow control valve 236 or some other directing arrangement can be used to direct the sample and non-magnetic beads to waste chamber 232 and then to capture chamber 234. A number of embodiments for rotationally dependent flow can be used. Further details relating to the use of flow control mechanisms are disclosed in commonly assigned co-pending U.S. patent application Ser. No. 09/997,741 entitled “Dual Bead Assays Including Optical Biodiscs and Methods Relating Thereto” filed Nov. 27, 2001, which is herein incorporated by reference in its entirety.

FIG. 35 is a perspective view of a disc including one embodiment of a fluidic circuit employed in conjunction with magnetic beads and the magnetic field generator 230 according to the present invention. FIG. 35 also shows the mixing chamber 164, the waste chamber 232, and the capture chamber 234. The magnetic field generator 230 is positioned over disc 110 and has a radius such that as disc 110 rotates, magnetic field generator 230 remains over mixing chamber 164, and is radially spaced from chambers 232 and 234. As with the prior embodiment discussed above, a magnetic field from the magnetic field generator 230 can be applied over the mixing chamber 164 to hold the dual bead complexes and/or unbound magnetic beads in place while additional material is allowed to enter the mixing chamber 164. The method of rotating the disc while holding magnetic beads in place with the magnetic field generator 230 may also be employed to aid in mixing of the assay solution within the mixing chamber 164 before the solution contained therein is directed elsewhere.

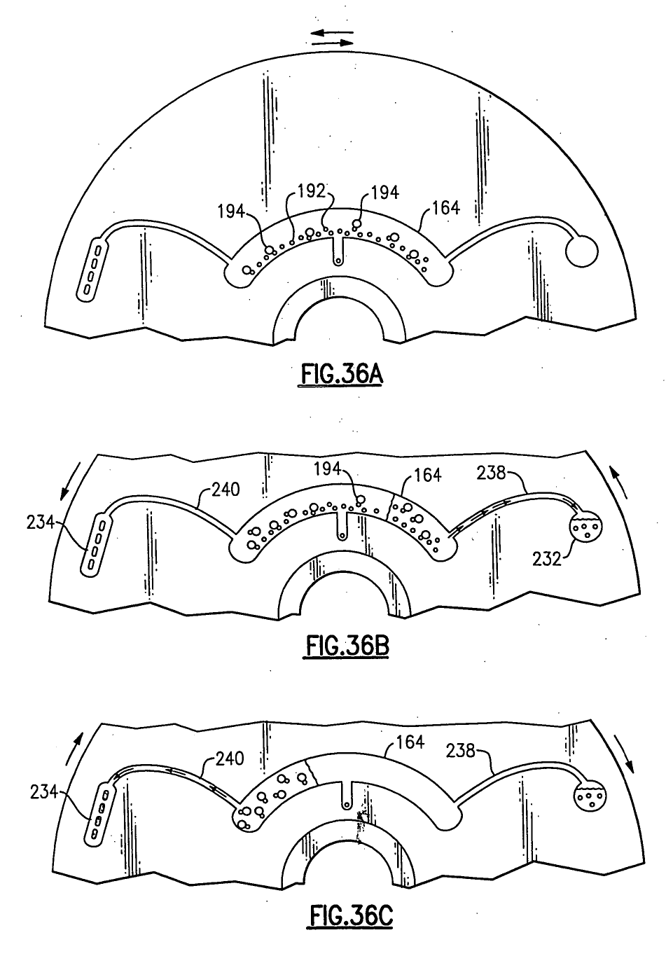

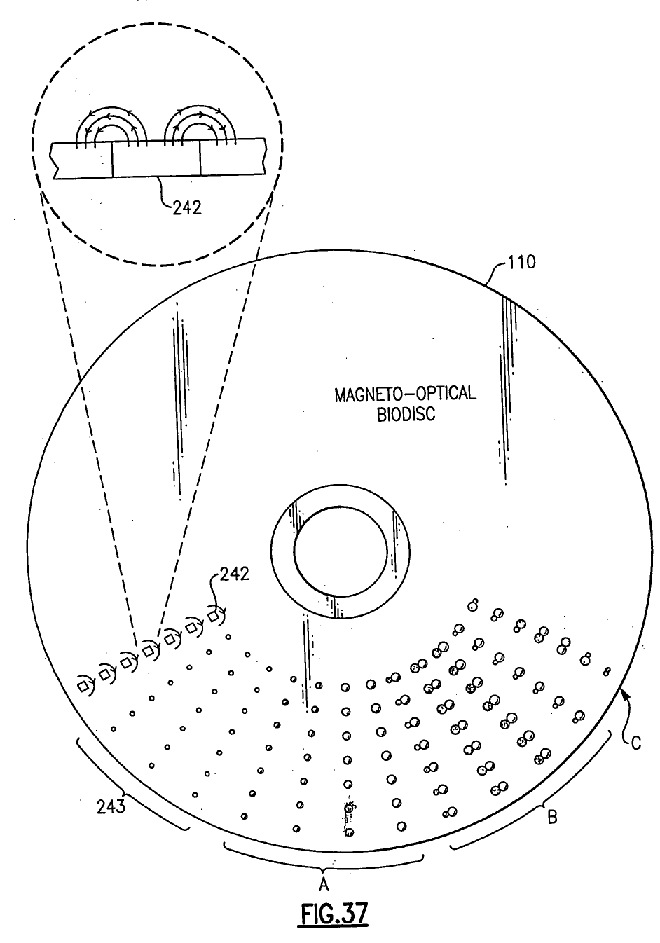

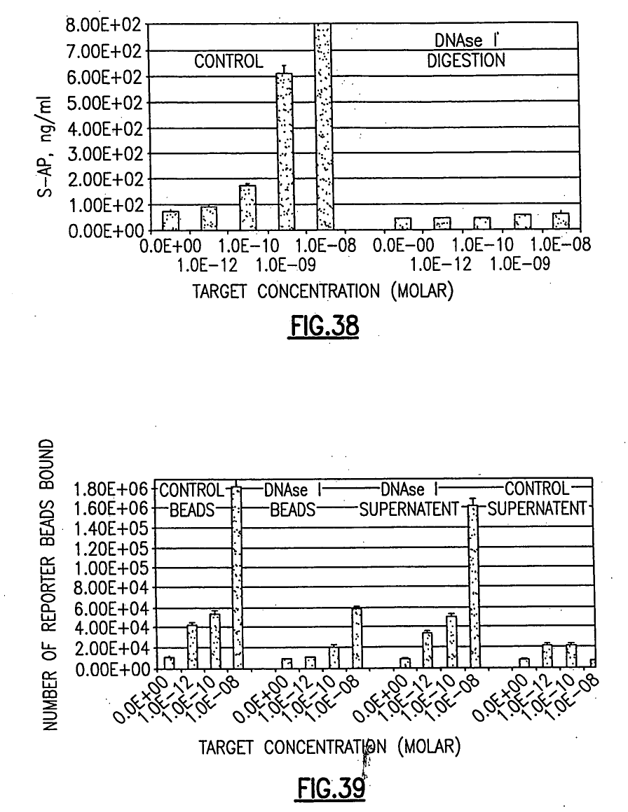

FIGS. 36A-36C are plan views illustrating a method of separation and detection for dual bead assays using the fluidic circuit shown in FIG. 35. FIG. 36A shows an unrotated optical disc with a mixing chamber 164 shaped as an annular sector that holding a sample with dual bead complexes 194 and various unbound reporter beads 192. The electromagnet is activated and the disc is rotated counter-clockwise (FIG. 36B), or it can be agitated at a lower rpm, such as 1× or 3×. Dual bead complexes 194, with magnetic capture beads, remain in mixing chamber 164 while the liquid sample and the unbound reporter beads 192 move in response to angular acceleration to a rotationally trailing end of mixing chamber 164. The disc is rotated in the counter-clockwise direction illustrated in FIG. 36B with sufficient speed to overcome capillary forces to allow the unbound reporter beads in the sample to move through a waste fluidic circuit 238 to waste chamber 232. At this stage in the process, the liquid will not move down the capture fluidic circuit 240 because of the physical configuration of the fluidic circuit as illustrated.

As illustrated next in FIG. 36C, the magnet is deactivated and the disc is rotated clockwise. Dual bead complexes 194 move to the opposite trailing end of the mixing chamber 164 in response to angular acceleration and then through a capture fluidic circuit 240 to the capture chamber 234. At this later stage in the process, the dual bead solution will not move down the waste fluidic circuit 238 due to the physical layout of the fluidic circuit, as shown. The embodiment shown in FIGS. 36A-36C thus illustrates directionally-dependent flow as well as rotational speed dependent flow.

In this embodiment and others in which a fluidic circuit is formed in a region of the disc, a plurality of regions can be formed and distributed about the disc, for example, in a regular manner to promote balance. Furthermore, as discussed above, instructions for controlling the rotation can be provided on the disc. Accordingly, by reading the disc, the disc drive can have instructions to rotate for a particular period of time at a particular speed, stop for some period of time, and rotate in the opposite direction for another period of time. In addition, the encoded information can include control instructions such as those relating to, for example, the power and wavelength of the light source. Controlling such system parameters is particularly relevant when fluorescence is used as a detection method.

In yet another embodiment, a passage can have a material or configuration that can seal or dissolve either under influence from a laser in the disc drive, or with a catalyst pre-loaded in the disc, or such a catalyst provided in the test sample. For example, a gel may solidify in the presence of a material over time, in which case the time to close can be set sufficiently long to allow the unbound capture beads to flow to a waste chamber before the passage to the waste chamber closes. Alternatively, the passage to the waste chamber can be open while the passage to the detection chamber is closed. After the unbound beads are directed to the waste chamber, the passage to the direction chamber is opened by energy introduced from the laser to allow flow to the detection chamber.

With reference now generally to FIG. 37, it is understood that magneto-optic recording is an optical storage technique in which magnetic domains are written into a thin film by heating it with a focused laser in the presence of an external magnetic field. The presence of these domains is then detected by the same laser from differences in the polarisation of the reflected light between the different magnetic domains in the layer (Kerr rotation). By switching either the magnetic field for constant high laser power, or modulating the laser power with a constant magnetic field, a data pattern can be written into the layer. Many magneto-optic storage systems have been introduced onto the market, including both computer data storage systems and audio systems (most notably MiniDisk).

Descriptions of the current status of this field can be found in “The Principles of Optical Disc Systems”, Bouwhuis et. al. 1985 (ISBN 0-85274-785-3); “Optical Recording, A Technical Overview” A. B. Marchant 1990 (ISBN 0-201-76247-1); and “The Physical Principles of Magneto-Optical Recording”, M. Mansuripur 1995 (ISBN 0521461243). All of these documents are herein incorporated by reference in their respective entireties.

Moving on now specifically to FIG. 37, there is illustrated yet another embodiment of the optical disc 110 for use with a multiplexing dual bead assay. In this case a disc, such as one used with a magneto-optical drive, has magnetic regions 242 that can be written and erased with a magnetic head. Hereafter this type of disc will generally be referred to as a “magneto-optical bio-disc” or an “MO bio-disc”. A magneto-optical disc drive, for example, can create magnetic regions 242 as small as 1 micron by 1 micron square. The close-up section of the magnetic region 242 shows the direction of the magnetic field with respect to adjacent regions.

The ability to write to small areas in a highly controllable manner to make them magnetic allows capture areas to be created in desired locations. These magnetic capture areas can be formed in any desired configuration or location in one chamber or in multiple chambers. These areas capture and hold magnetic beads when applied over the disc. The domains can be erased if desired, thereby allowing them to be made non-magnetic and allowing the beads to be released.

In one configuration of a magnetic bead array according to this aspect of the present invention, a set of three radially oriented magnetic capture regions 243 are shown, by way of example, with no beads attached to the magnetic capture regions in the columns illustrated therein. With continuing reference to FIG. 37, there is shown a set of four columns in Section A with individual magnetic beads magnetically attached to the magnetic areas in a magnetic capture region. Another set of four columns arrayed in Section B is shown after binding of reporter beads to form dual bead complexes attached to specific magnetic areas, with different columns having different types of reporter beads. As illustrated in Section B, some of the reporter beads utilized vary in size to thereby achieve the multiplexing aspects of the present invention as implemented on a magneto-optical bio-disc or MO medical disc. In Section C, a single column of various dual bead complexes is shown as another example of multiplexing assays employing various bead sizes individually attached at separate magnetic areas.

In a method of using such a magneto-optical bio-disc or MO medical disc, the write head in an MO drive is employed to create magnetic areas, and then a sample can be directed over that area to capture magnetic beads provided in the sample. After introduction of the first sample set, other magnetic areas may also be created and another sample set can be provided to the newly created magnetic capture region for detection. Thus detection of multiple sample sets may be performed on a single disc at different time periods. The magneto-optical drive also allows the demagnetization of the magnetic capture regions to thereby release and isolate the magnetic beads if desired. Thus this system provides for the controllable capture, detection, isolation, and release of one or more specific target molecules from a variety of different biochemical, chemical, or biological samples.

As described above, a sample can be provided to a chamber on a disc. Alternatively, a sample could be provided to multiple chambers that have sets of different beads. In addition, a series of chambers can be created such that a sample can be moved by rotational motion from one chamber to the next, and separate tests can then be performed in each chamber.

With such an MO bio-disc, a large number of tests can be performed at one time and can be performed interactively. In this manner, when a test is performed and a result is obtained, the system can be instructed to create a new set of magnetic regions for capturing the dual bead complex. Regions can be created one at a time or in large groups, and can be performed in successive chambers that have different pre-loaded beads. Other processing advantages can be obtained with an MO bio-disc that has writeable magnetic regions. For example, the “capture agent” is essentially the magnetic field created by the magnetic region on the disc and therefore there is no need to add an additional biological or chemical capture agent.

Instructions for controlling the locations for magnetic regions written or erased on the MO bio-disc, and other information such as rotational speeds, stages of rotation, waiting periods, wavelength of the light source, and other parameters can be encoded on and then read from the disc itself. As would be readily apparent to one of ordinary skill in the art given the disclosure provided herein, the MO bio-disc illustrated in FIG. 37 may include any of the fluidic circuits, mixing chambers, flow channels, detection chambers, inlet ports, or vent ports employed in the reflective and transmissive discs discussed above. Illustrative examples of the use of the MO bio-disc according to this aspect of the present are provided below in Examples 6 and 7.

Thus in summary, the following embodiments of the magneto-optical aspects of the present invention have been contemplated by the inventors and are herein described in detail. Firstly, there is provided a method of performing a genetic dual bead assay in association with a magneto-optical bio-disc. This method includes the steps providing a plurality of magnetic capture beads having covalently attached transport probes, providing a plurality of reporter beads having covalently attached specific sequences of DNA, preparing a sample containing target DNA molecules to be tested for DNA sequences complementary to the specific DNA sequences, and loading the capture beads into a magneto-optical bio-disc via an inlet port provided therein. The magneto-optical bio-disc has a magnetic capture layer. This method further includes loading the sample and the plurality of reporter beads into the bio-disc, rotating the bio-disc to facilitate hybridization of any target DNA present in the sample to the specific sequences of DNA on the reporter beads and to the transport probes to form dual bead complexes, interrogating a number of the magnetic capture beads with an incident beam of radiant energy to determine whether each of the number of magnetic capture beads has formed a dual bead complex, magnetizing specific regions of the magnetic capture layer to bind thereto a plurality of the dual bead complexes, and quantitating the plurality of the dual bead complexes.

The method may include the further steps of rotating the disc to direct any unbound beads into a waste chamber and then de-magnetizing the specific regions of the magnetic capture layer to thereby release a number of the plurality of the dual bead complexes. Thereafter the disc may be rotated to direct the released number of dual bead complexes to an analysis area for further processing so that the released number of dual bead complexes are sequestered in the analysis area. The analysis area may be an analysis chamber having agents that react with the sequestered dual bead complexes.

According to a second embodiment of the magneto-optical aspects of the present invention there is provided another method of performing a dual bead assay in association with a magneto-optical bio-disc. This other method includes the steps of providing a plurality of magnetic capture beads having attached transport probes, providing a plurality of reporter beads having attached signal probes, and loading the capture beads into a magneto-optical bio-disc via an inlet port provided therein. The magneto-optical bio-disc has a magnetic capture layer. This second method further includes loading a sample containing a target and the plurality of reporter beads into the bio-disc, rotating the bio-disc to facilitate binding of the target and the reporter beads to the magnetic capture beads to form dual bead complexes, interrogating a number of the magnetic capture beads with an incident beam of radiant energy to determine whether each of the number of magnetic capture beads has formed a dual bead complex, magnetizing specific regions of the magnetic capture layer to bind thereto a plurality of the dual bead complexes, and quantitating the plurality of the dual bead complexes.

This method may similarly include the further step of rotating the disc to direct any unbound beads into a waste chamber and then de-magnetizing the specific regions of the magnetic capture layer to thereby release a number of the plurality of the dual bead complexes. It is also an aspect of this method to then rotate the disc to direct the released number of dual bead complexes to an analysis area for further processing so that the released number of dual bead complexes are sequestered in the analysis area. The analysis area may include a reaction chamber having agents that react with the sequestered dual bead complexes.

In accordance with a third embodiment of the magneto-optical aspects of the present invention there is provided a method of performing a multiplexed dual bead assay in association with a magneto-optical bio-disc. This multiplexing method includes the steps of (1) providing at least two groups of differently sized magnetic capture beads, each group having magnetic capture beads of the same size and having a different specific type of transport probe associated with each group; (2) providing a plurality of reporter beads having attached at least two different types of signal probes; and (3) loading the capture beads into a magneto-optical bio-disc via an inlet port provided therein. As in the above MO bio-disc methods, this magneto-optical bio-disc has a magnetic capture layer. The method also includes (4) loading a sample containing at least one target and the plurality of reporter beads into the bio-disc; (5) rotating the bio-disc to facilitate binding of the target and the reporter beads to the magnetic capture beads to form dual bead complexes; (6) interrogating a number of the magnetic capture beads with an incident beam of radiant energy to determine whether each of the number of magnetic capture beads has formed a dual bead complex; and (7) determining the size of the magnetic bead in the dual bead complex. This particular method concludes with the steps of (8) magnetizing specific regions of the magnetic capture layer to bind thereto a plurality of the dual bead complexes; and (9) quantitating the plurality of the dual bead complexes.

According to one aspect of this specific method, the step of quantitating may advantageously include quantitating the plurality of the dual bead complexes according to the size of the magnetic capture bead. The method may include the further step of rotating the disc to direct any unbound beads into a waste chamber and then de-magnetizing the specific regions of the magnetic capture layer to thereby release a number of the plurality of the dual bead complexes containing same-sized magnetic capture beads. The method may further include rotating the disc to direct the released number of same-sized dual bead complexes to an analysis area for further processing so that the released number of same-sized dual bead complexes are sequestered in the analysis area. The analysis area may include a reaction chamber having agents that react with the sequestered same-sized dual bead complexes. In one particular embodiment hereof, the signal probe is a specific sequence of DNA.

According to yet a fourth embodiment of the magneto-optical aspects of the present invention there is provided another principal method of performing a multiplexed dual bead assay in association with a magneto-optical bio-disc. This additional dual bead multiplexing method includes the steps of (1) providing at least two groups of different types of reporter beads, each group having reporter beads of the same type and having a different specific type of signal probe associated with each group; (2) providing a plurality of magnetic capture beads having different types of transport probes attached thereto; and (3) loading the capture beads into a magneto-optical bio-disc via an inlet port provided therein. As in the above MO bio-diac methods, this particular magneto-optical bio-disc has a magnetic capture layer. The method continues with the additional steps of (4) loading a sample to be tested for at least one target and the plurality of reporter beads into the bio-disc; (5) rotating the bio-disc to facilitate binding of any target present in the sample to the reporter beads and to the magnetic capture beads to form dual bead complexes; and (6) interrogating a number of the reporter beads with an incident beam of radiant energy to determine whether each of the number of reporter beads has formed a dual bead complex. This particular embodiment of the present method then concludes with (7) determining the type of the reporter bead in the dual bead complex; (8) magnetizing specific regions of the magnetic capture layer to bind thereto a plurality of the dual bead complexes; and (9) quantitating the plurality of the dual bead complexes.

In one specific embodiment hereof, the step of quantitating includes quantitating the plurality of the dual bead complexes according to the type of reporter bead. The method may further include the further step of rotating the disc to direct any unbound beads into a waste chamber and then, if desired, de-magnetizing the specific regions of the magnetic capture layer to thereby release a number of the plurality of the dual bead complexes containing same-type reporter beads. The further step of rotating the disc to direct the released number of same-type dual bead complexes to an analysis area for further processing so that the released number of same-type dual bead complexes are sequestered in the analysis area may also be performed. As with the above methods, the analysis area may include a reaction chamber having agents that react with the sequestered same-type dual bead complexes.

The present invention further contemplates an optical bio-disc used to perform any of the above methods, and an optical bio-disc used to analyze any the dual bead complexes prepared the methods discussed in connection with FIGS. 11A-1, 11A-2, 11B-1, 11B-2 12A-1, 12A-2, 12B-1, or 12B-2.

Use of Dissociation Agents to Increase Assay

Sensitivity and Decrease Non-specific Bead Binding

Moving along to FIG. 38 now, there is shown a bar graph presentation demonstrating the DNAseI digestion efficiency in the absence of reporter beads. In this experiment, biotinylated target DNA 202 was hybridized to transport probes 198 on the magnetic capture bead 190 as illustrated above in FIG. 8A. After hybridization, streptavidinated alkaline phosphatase (S-AP) was added to the assay mix and allowed to bind with the biotin on the target DNA. Following a series of wash steps, an S-AP chromagen substrate was added to an aliquot of the assay solution and the amount of bound target was quantified calorimetrically using a spectrophotometer. At the same time, an aliquot of equal volume to that taken above was incubated in buffer containing DNAseI. After incubation, the assay mix was washed and S-AP was added to the solution and allowed to bind to residual targets that were not digested by DNAseI. The observations showed a high DNAse digestion activity as manifested in the difference in signal between the control and the DNAseI digestion treatments. Details regarding an experiment similar to the one discussed here is described in detail below in Example 3.

Referring now to FIG. 39, there is shown a bar graph illustration of data collected from an experiment similar to that discussed in FIG. 38. In this experiment, dual bead complexes were used instead of the magnetic capture bead alone as described in FIG. 38. In this case, the target 202 is situated between the capture bead 190 and reporter bead 192 as shown above in FIG. 10A. After formation of the dual bead complex, as described in FIG. 11A-1, Steps I-V, the amount of reporter beads in the supernatant collected in Step IV (FIG. 11A-1) and the reporter beads bound to the capture beads in the assay mix shown in Step V (FIG. 11A-1) were quantitated using a fluorimeter. DNAseI was then added to the assay mix and allowed to cleave the target bound to the probes in the dual bead complex, as represented in Step VI in FIG. 11A-2, thereby releasing the reporter bead from the magnetic capture beads. The next step involved the isolation of the capture beads and collection of the supernatant containing newly released fluorescent reporter beads as shown in Step VII in FIG. 11A-2. The signal from the reporter beads in the supernatant was quantified using a fluorimeter. The undissociated reporter beads were also quantified by a fluorimeter. Data collected from this experiment shows the DNAseI enzyme digestion was not as efficient in a dual bead complex relative to that in a single bead set-up as described in FIG. 38. The decrease in DNAse digestion activity may be due to steric hindrance from the beads in the dual bead complex blocking DNAse access to the target. Experimental details regarding the use of restriction enzyme digestion as implemented in a dual bead assay is discussed in further detail below in Example 3. The fluorescent reporter beads collected in this assay may also be quantified using an optical bio-disc or medical CD with a fluorescent type optical disc reader or any similar device as discussed above.

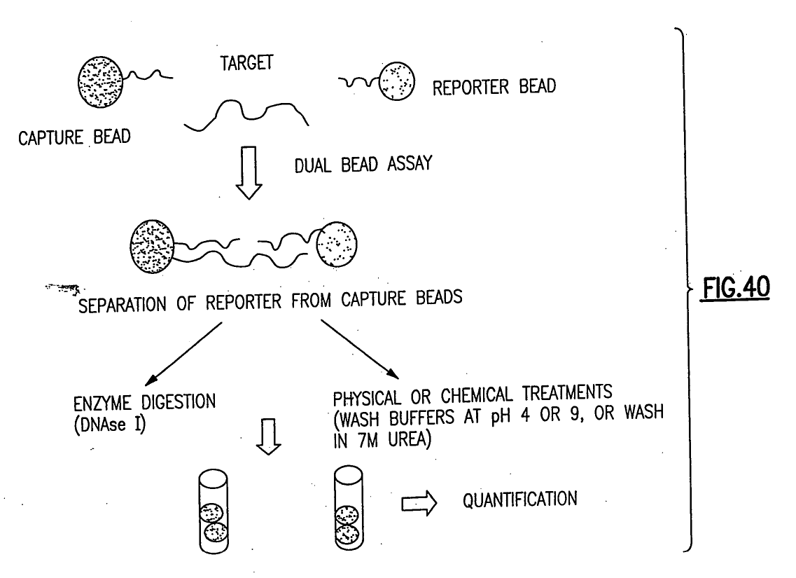

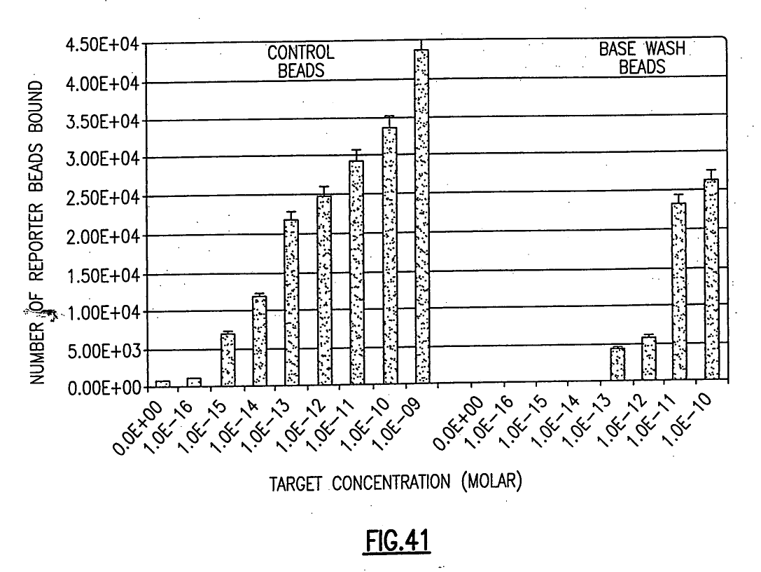

The sensitivity of any assay depends on the ratio of signal over noise. The sensitivity of the dual bead assay relies on the minimization of non-specific binding between capture beads and reporter beads. The non-specific interactions between the dual beads in the absence of targets are so stable that stringent washing cannot eliminate them. The contribution of the non-specific dual beads, however, can be negated by the exclusive detection and quantification of target-mediated dual beads. As shown in FIG. 40, the dual bead complexes can be separated by enzyme digestion (DNAse, restriction enzymes) or by chemical and physical treatments (heat, urea, base, or acid treatment). Furthermore, the quantification of reporter beads in the presence of a large excess of unbound capture beads may reduce the sensitivity of the detection assay. Therefore, the separation of reporter beads from capture beads will facilitate the quantification of reporter beads by the bio-disc reader and thus increase the sensitivity of the assay.

With more particular reference now to FIG. 40, there is a schematic representation of separation of reporter beads from capture beads in a dual bead complex by enzyme digestion and physical or chemical treatments. FIG. 40 shows a summary of the dual bead formation and dissociation using various dissociation agents as discussed in conjunction with FIGS. 11A-1, 11A-2, 12A-1, and 12A-2. After the released reporter beads are collected, the beads are quantified by any one of several methods including a fluorimeter, a fluorimager, a fluorescent type optical disc reader system, a CD-R type optical disc system or any device capable of detecting micro-spheres or fluorescence. The current preferred method of detection is the use of an optical bio-disc or medical CD system as discussed in detail in connection with FIGS. 1, 2, 3A, 3B, 3C, 4A, 4B, and 4C.

With reference next to FIG. 41, there is a bar graph presentation showing separation of reporter beads from capture beads using high pH washes at various target concentrations similar to that described in FIG. 39. The dual bead complex is formed as described in Steps I-VI in FIG. 12A-1. Once the dual beads are formed and isolated, a strong base is added to the dual bead solution as shown in Step VI (FIG. 12A-1). After a brief incubation, the dual beads are dissociated by the actions of the base that disrupts the hydrogen bonding between the target and the probes thereby releasing the fluorescent reporter beads and the magnetic capture beads from the dual bead complex as shown in Step VII (FIG. 12A-2). The next step is to isolate the capture beads as described in Step VIII of FIG. 12A-2. The isolated capture beads will also contain non-dissociated dual bead complexes. This isolate was quantified by a fluorimeter in this particular experiment. The data shown in FIG. 41 illustrate 100% dissociation of dual bead complexes at target concentrations from 1×10¹⁶M to 1×10−14M. Details of an experiment performed using base as a dissociation agent is described in detail below in Example 4. The reporter beads may also be quantified by any one of several methods including a fluorimager, a fluorescent type optical disc reader system, a CD-R type optical disc system or any device capable of detecting microspheres or fluorescence. The preferred method of detection is the use of an optical bio-disc or medical CD system as discussed in detail in conjunction with FIGS. 1, 2, 3A, 3B, 3C, 4A, 4B, and 4C.

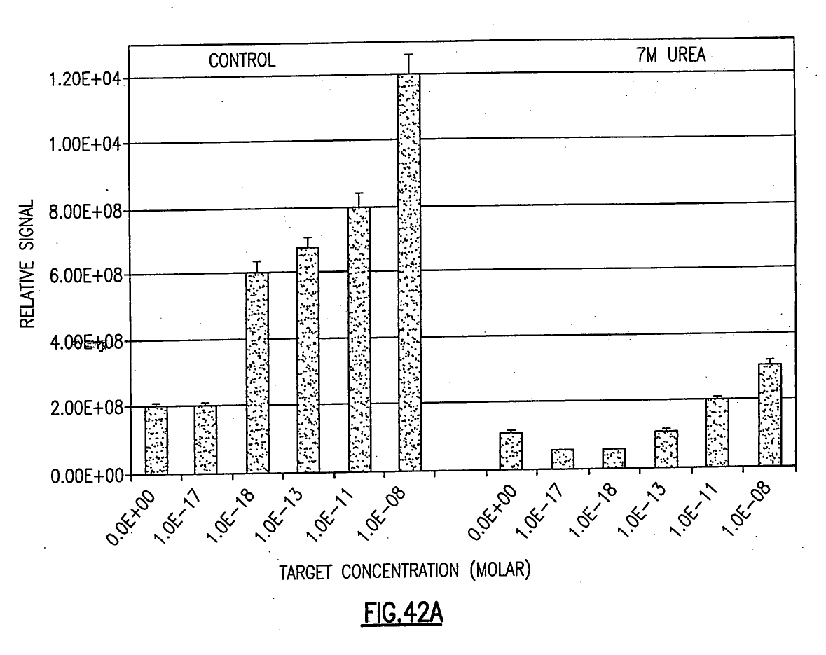

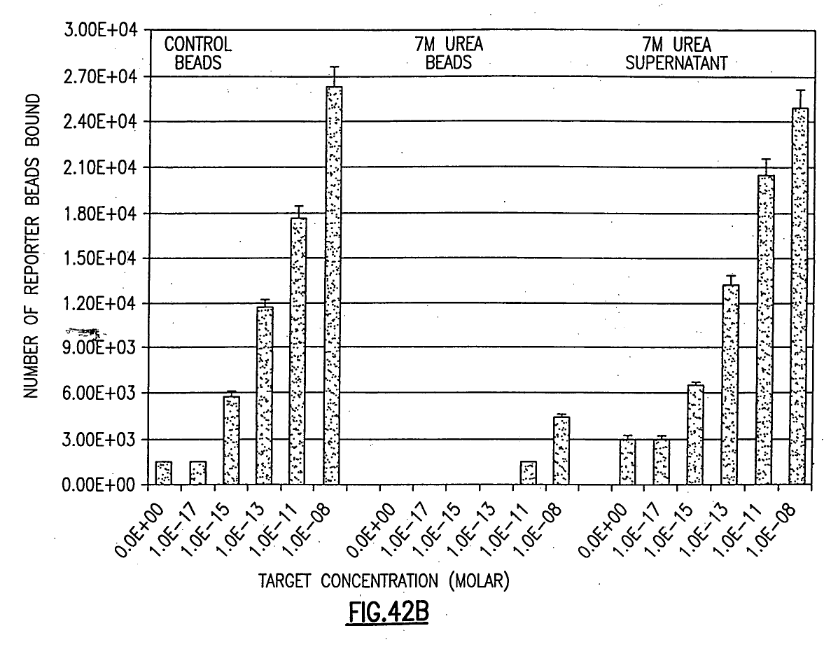

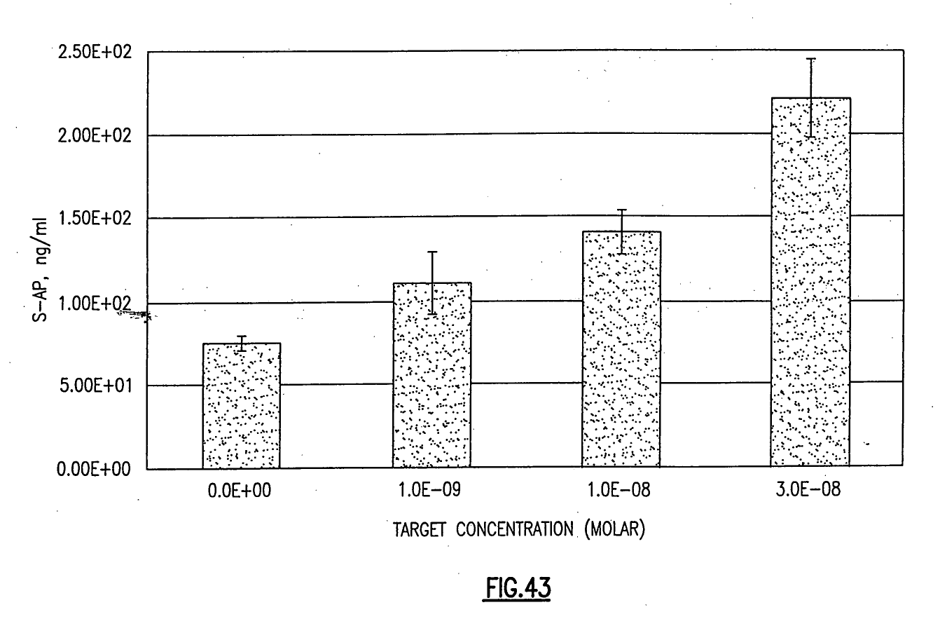

Referring now to FIG. 42A, there is a bar graph illustration of data collected from an experiment using urea as a denaturing or dissociation agent. Details of this experiment are similar to those discussed in conjunction with FIG. 38. As in FIG. 38, biotinylated target DNA 202 was hybridized to transport probes 198 on the magnetic capture bead 190 as illustrated above in FIG. 8A. After hybridization, streptavidinated alkaline phosphatase (S-AP) was added to the assay mix and allowed to bind with the biotin on the target DNA 202. Following a series of wash steps, an S-AP chromagen substrate was added to an aliquot of the assay solution and the amount of bound target was quantified calorimetrically using a spectrophotometer. At the same time, an aliquot of equal volume to that taken above was incubated in buffer containing 7M urea. After incubation, the assay mix was washed and S-AP was added to the solution and allowed to bind to residual targets, still bound to transport probes on the capture bead, that were not denatured by the 7M urea. The results thereof showed a relatively efficient denaturation activity as revealed by the difference in signal between the control and the 7M urea treatments. Details regarding an experiment similar to the one discussed here is described in detail below in Example 4.

Referring next to FIG. 42B, there is a bar graph illustrating data collected from an experiment using urea as a dissociation agent in a dual bead assay. Details of this experiment are similar to that discussed in conjunction with FIG. 39. As in FIG. 39, dual bead complexes were used instead of the magnetic capture bead alone as described in FIG. 42A. In this case, the target is situated between the capture and reporter beads as shown above in FIG. 10A. After formation of the dual bead complex, as described in FIG. 12A-1, Steps I-VI, the amount of reporter beads 192 in the supernatant collected in Step V (FIG. 12A-1) and the reporter beads 192 bound to the capture beads 190 in the assay mix shown in Step VI (FIG. 12A-1) were quantitated using a fluorimeter. A predetermined amount of urea was then added to make the final urea concentration in the assay solution 7M. This resulted in denaturation of the target bound to the probes in the dual bead complex, as represented in Step VII in FIG. 12A-2, thus releasing the reporter bead from the magnetic capture beads. The next step involved the isolation of the capture beads 190 and collection of the supernatant containing newly released fluorescent reporter beads 192 as shown in Step VIII in FIG. 12A-2. The signal from the reporter beads in the supernatant was quantified using a fluorimeter. The undissociated reporter beads were also quantified by a fluorimeter. Data collected from this experiment shows the 7M urea denaturation was more efficient in a dual bead complex assay relative to using DNAseI as a dissociation agent as described in FIG. 39. The increase in dissocaition may be due to the lack of steric hindrance from the beads in the dual bead complex since urea is a significantly smaller molecule than DNAse. Experimental details regarding the use of 7M urea denaturation as implemented in a dual bead assay is discussed in further detail in Example 4. The fluorescent reporter beads collected in this assay may also be quantified using an optical bio-disc or medical CD with a fluorescent type optical disc reader or any similar device as discussed above.

Use of DNA Denaturing Agents to Improve DNA Target Detection

It is a principal aspect of the invention to further modify the dual bead assays to detect medical targets. In real samples, the DNA targets are double-stranded and very long. The ability of the dual bead assay, as well as for any other DNA diagnostic assays, to detect sequences of clinical interest within the whole genome relies first on the specificity of the probes for the sequence of interest and second on the use of very strong detergent to keep the DNA target in the denatured, single-stranded, form for capture.

The success of the dual bead assays in detecting sequences of clinical interest relies primarily on the design of the probes. Given the complexity and degeneracy of the human genome, the probes designed to detect sequences of clinical interest have to be unique to the diagnostic sequence and yet common enough to recognize mutants of the sequences. The design of the probes using computer software allows comparison of sequences to existing sequences in the data bank such as Blast search. Once probes specific for the sequence of interest have been designed, the major modification introduced to the dual bead assays includes the use of a denaturing agent in the hybridization buffer to prevent re-annealing of complementary sequences of the target DNA. This allows hybridization between the target and probes.

The present invention is also addressed at implementing the methods recited above on to an analysis disc, modified optical disc, medical CD, or a bio-disc. A bio-disc or medical CD drive assembly, such as those discussed above with reference to FIGS. 1 and 2, may be employed to rotate the disc, read and process any encoded information stored on the disc, and analyze the DNA samples in the flow channel of the bio-disc or medical CD. The bio-disc drive is thus provided with a motor for rotating the bio-disc, a controller for controlling the rate of rotation of the disc, a processor for processing return signals form the disc, and an analyzer for analyzing the processed signals. The rotation rate of the motor is controlled to achieve the desired rotation of the disc. The bio-disc drive assembly may also be utilized to write information to the bio-disc either before, during, or after the medical test material in the flow channel and target zones is interrogated by the read beam of the drive and medically diagnosed by the analyzer. In this embodiment of the present invention, the analyzer may advantageously include specialized diagnostic software to thereby provide a medical expert system. The bio-disc may similarly include corresponding medical expert system software and encoded information for controlling the rotation rate of the disc, providing processing information specific to the type of DNA test to be conducted, and for displaying the results on a monitor associated with the bio-drive.

In a preferred embodiment of this invention, guanidine isothiocyanate is used as a typical denaturing agent. Data collected from an experiment using 1.5M guanidine isothiocyanate as denaturing agent is illustrated in FIG. 43. The experimental procedure followed for this experiment is described in detail below in Example 5. FIG. 43 further illustrates that the assay sensitivity is significantly increased if an appropriate amount of denaturing agent is used in a hybridization experiment. In this particular assay, biotinylated target DNA 202 was hybridized to transport probes 198, in the presence of 1.5M guanidine isothiocyanate, on the magnetic capture bead 190 as illustrated above in FIG. 8A. After hybridization, streptavidinated alkaline phosphatase (S-AP) was added to the assay mix and allowed to bind with the biotin on the target DNA 202. Following a series of wash steps, an S-AP chromagen substrate was added to the assay solution, ample time was allotted for color development, and the amount of bound target was quantified calorimetrically using a spectrophotometer.

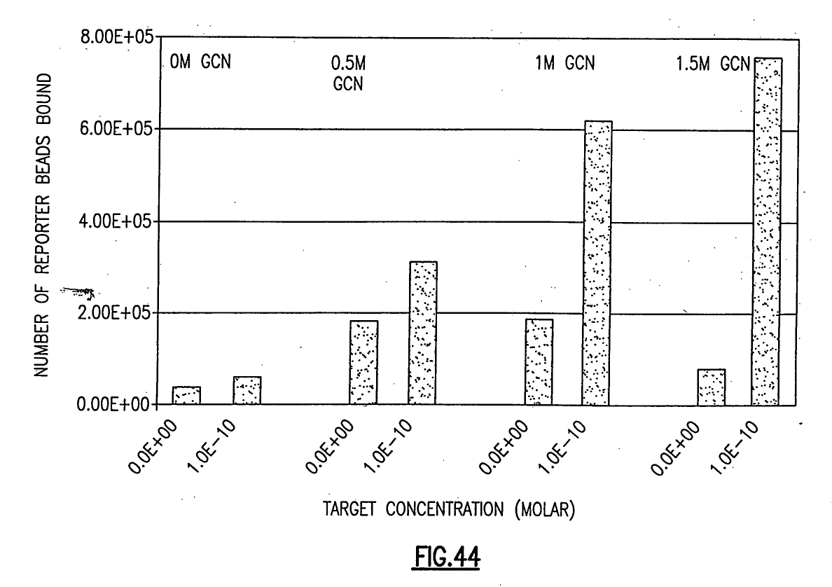

An appropriate amount of guanidine isothiocyanate is necessary to prevent re-annealing of the complementary sequences of the target DNA while allowing hybridization between the target and the probes. At high concentrations, however, guanidine isothiocyanate prevents any hybridization. To determine the appropriate buffer concentration of guanidine isothiocyanate for use in a dual bead assay, a titration of guanidine isothiocyanate was performed. The data from this titration experiment is shown in FIG. 44. As illustrated in FIG. 44, the optimal hybridization buffer concentration of guanidine isothiocyanate is 1.5M since the addition of 1.5M guanidine isothiocyanate showed the highest difference in signal between the 0.0M and 1.0×10⁻¹⁰M target concentration.

### EXPERIMENTAL DETAILS

While this invention has been described in detail with reference to the drawing figures, certain examples and further illustrations of the invention are presented below.

### Example 1

The two-step hybridization method demonstrated in FIGS. 12A-1 and 12A-2 was used in performing the dual bead assay of this example.

A. Dual Bead Assay

In this example, the dual assay in carried out to detect the gene sequence DYS that is present in male but not in female. The assay is comprised of 3μ magnetic and capture beads coated with covalently attached capture or transport probe; 2.1μ fluorescent reporter beads coated with a covalently attached sequence specific for the DYS gene, and target DNA molecule containing DYS sequences. The target DNA is a synthetic 80 oligonucleotide sequence. The transport probe and reporter probes are 40 nucleotides in length and are complementary to DYS sequence but not to each other.

The specific methodology employed to prepare the assay involved treating 1×10⁷ capture beads and 2×10⁷ reporter beads in 100 microgram per milliliter Salmon Sperm DNA for 1 hr. at room temperature. This pretreatment will reduce non-covalent binding between the capture and reporter beads in the absence of target DNA as shown in FIG. 38. The capture beads were concentrated magnetically with the supernatant being removed. A 100 microliter volume of the hybridization buffer (0.2 M NaCl, 1 mM EDTA, 10 mM MgCl₂, 50 mM Tris HCl, pH 7.5, and 5× Denhart's mixture, 10 microgram per milliliter denatured salmon sperm DNA) were added to the capture beads and the beads were re-suspended. Various concentrations of target DNA ranging from 1, 10, 100, 1000 femtomoles were added while mixing at 37° C. for 2 hours. The beads were magnetically concentrated and the supernatant containing target DNA was removed. A 100 microliter volume of wash buffer (145 mM NaCl, 50 mM Tris pH 7.5, 0.1% SDS, 0.05% Tween, 0.25% NFDM, 10 mM EDTA) was added and the beads were re-suspended. The beads were magnetically concentrated and the supernatant was again removed. The wash procedure was repeated twice.

A 2×10⁷ amount of reporter beads in 100 microliter hybridization buffer (0.2 M NaCl, 1 mM EDTA, 10 mM MgCl₂, 50 mM Tris HCl, pH 7.5, and 5× Denhart's mixture, 10 microgram per milliliter denatured salmon sperm DNA) were then added to washed capture beads. The beads were re-suspended and incubated while mixing at 37° C. for an additional 2 hours. After incubation the capture beads were concentrated magnetically, and the supernatant containing unbound reporter beads were removed. A 100 microliter volume of wash buffer (145 mM NaCl, 50 mM Tris pH 7.5, 0.1% SDS, 0.05% Tween, 0.25% NFDM, 10 mM EDTA) was added and the beads were re-suspended. The beads were magnetically concentrated and the supernatant was again removed. The wash procedure was repeated twice.

After the final wash, the beads were re-suspended in 20 microliters of binding buffer (50 mM Tris, 200 mM NaCl, 10 mM MgCl₂, 0.05% Tween 20, 1% BSA). A 10 microliter volume was loaded on to the disc that was prepared as described below in Part B of this example.

B. Preparation of the Disc

A gold disc was coated with maleic anhydride polystyrene. An amine DNA sequence complementary to the reporter probes (or capture agent) was immobilized on to the discrete reaction zones on the disc. Prior to sample injection, the channels were blocked with a blocking buffer (50 mM Tris, 200 mM NaCl, 10 mM MgCl₂, 0.05% Tween 20, 1% BSA, 1% sucrose) to prevent non-covalent binding of the dual bead complex to the disc surface. A perspective view of the disc assembly showing capture agents 220, the capture zones 170, and fluidic circuits as employed in the present invention is illustrated in detail in FIGS. 25A-25D. Alternatively, if the reporter beads are coated with Streptavidin, a capture zone could be created with the capture agent such as BSA Biotin which could be immobilized on to the disc (pretreated with Polystyrene) by passive absorption. A perspective view of the disc assembly showing the use of biotin capture agents is presented in FIGS. 26A-26D. Various methods for use in this type of anchoring of beads onto the disc are also shown in FIGS. 15A-15B, 17, 19A-19C, and 23A-23B.

C. Capture of Dual Bead Complex Structure on the Disc

A 10 microliter volume of the dual bead mixture prepared as described in Part A above was loaded in to the disc chamber and the injection ports were sealed. To facilitate hybridization between the reporter probes on the reporter beads and the capture agents, the disc was centrifuged at low speed (less than 800 rpm) upto 15 minutes. The disc was read in the CD reader at the speed 4× (approx. 1600 rpm) for 5 minutes. Under these conditions, the unbound magnetic capture beads were centrifuged away from the capture zone. The magnetic capture beads that were in the dual bead complex remained bound to the reporter beads in the capture zone. The steps involved in using the disc to capture and analyze dual bead complexes are presented in detail in FIGS. 25A-25D, 26A-26D, and 27A-27D.

D. Quantification of the Dual Bead Complex Structures

The amount of target DNA captured could be enumerated by quantifying the number of capture magnetic beads and the number of reporter beads since each type of bead has a distinct signature.

### Example 2

A. Dual Bead Assay Multiplexing

In this example, the dual bead assay is carried out to detect two DNA targets simultaneously. The assay is comprised of 3μ magnetic capture bead. One population of the magnetic capture bead is coated with capture or transport probes 1 which are complementary to the DNA target 1, another population of magnetic capture beads is coated with capture or transport probes 2 which are complementary to the DNA target 2. Alternatively two different types of magnetic capture beads may be used. There are two distinct types of reporter beads in the assay. The two types may differ by chemical composition (for example Silica and Polystyrene) and/or by size. Various combinations of beads that may be used in a multiplex dual bead assay format are depicted in FIG. 32. One type of reporter bead is coated with reporter probes 1, which are complementary to the DNA target 1. The other reporter beads are coated with reporter probes 2, which are complementary to the DNA target 2. Again the transport probes and the reporter probes are complementary to the respective targets but not to each other.

The specific methodology employed to prepare the dual bead assay multiplexing involved treating 1×10⁷ capture beads and 2×10⁷ reporter beads in 100 μg/ml salmon sperm DNA for 1 hour at room temperature. This pretreatment will reduce non-covalent binding between the capture and reporter beads in the absence of target DNA. The capture beads were concentrated magnetically with the supernatant being removed. A 100 microliter volume of the hybridization buffer (0.2 M NaCl, 1 mM EDTA, 10 mM MgCl₂, 50 mM Tris HCl, pH 7.5, and 5× Denhart's mixture, 10 microgram per milliliter denatured salmon sperm DNA) were added and the beads were re-suspended. Various concentrations of target DNA ranging from 1, 10, 100, 1000 femto moles were added to the capture beads suspension. The suspension was incubated while mixing at 37° C. for 2 hours. The beads were magnetically concentrated and the supernatant containing target DNA was removed. A 100 microliter volume of wash buffer (145 mM NaCl, 50 mM Tris pH 7.5, 0.1% SDS, 0.05% Tween, 0.25% NFDM, 10 mM EDTA) was added and the beads were re-suspended. The beads were magnetically concentrated and the supernatant was again removed. The wash procedure was repeated twice.

A 2×10⁷ amount of reporter beads in 100 microliter hybridization buffer (0.2 M NaCl, 1 mM EDTA, 10 mM MgCl₂, 50 mM Tris HCl, pH 7.5, and 5× Denhart's mixture, 10 microgram per milliliter denatured salmon sperm DNA) were then added to washed capture beads. The beads were re-suspended and incubated while mixing at 37° C. for an additional 2 hours. After incubation the capture beads were concentrated magnetically, and the supernatant containing unbound reporter beads were removed. A 100 microliter volume of wash buffer (145 mM NaCl, 50 mM Tris pH 7.5, 0.1% SDS, 0.05% Tween, 0.25% NFDM, 10 mM EDTA) was added and the beads were re-suspended. The beads were magnetically concentrated and the supernatant was again removed. The wash procedure was repeated twice.

After the final wash, the beads were re-suspended in 20 microliters of binding buffer (50 mM Tris, 200 mM NaCl, 10 mM MgCl₂, 0.05% Tween 20, 1% BSA). A 10 microliter volume of this solution was loaded on to the disc that was prepared as described in below in Part B of this example.

B. Disc Preparation

A gold disc was coated with maleic anhydride polystrene as described. Distinct reaction zones were created for two types of reporter beads. Each reaction zone consisted of amine DNA sequences complementary to the respective reporter probes (or capture agents). Prior to sample injection, the channel were blocked with a blocking buffer (50 mM Tris, 200 mM NaCl, 10 mM MgCl₂, 0.05% Tween 20, 1% BSA, 1% sucrose) to prevent non-covalent binding of the dual bead complex to the disc surface. Alternatively, magnetic beads employed in a multiplexing dual bead assay format may be detected using a magneto-optical disc and drive. The chemical reaction zones, in the magnetic disc format, are replaced by distinctly spaced magnetic capture zones as discussed in conjunction with FIG. 37.

C. Capture of Dual Bead Complex Structure on the Disc

A 10 microlitre volume of the dual bead mixture prepared as described above in Part A of this example, was loaded in to the disc chamber and the injection ports were sealed. To facilitate hybridization between the reporter probes on the reporter beads and the capture agents, the disc was centrifuged at low speed (less than 800 rpm) for up to 15 minutes. The disc was read in the CD reader at the speed 4× (approx. 1600 rpm) for 5 minutes. Under these conditions, the unbound magnetic capture beads were centrifuged to the bottom of the channels. The reporter beads bound to the capture zone via hybridization between the reporter probes and their complementary agent.

D. Quantification of the Dual Bead Complex Structures

The amount of target DNA 1 and 2 captured could be enumerated by quantifying the number of the respective reporter beads in the respective reaction zones.

### Example 3

After formation of the dual bead complexes, as discussed in connection with FIG. 12A-1 the reporter beads can be separated from the capture beads in a DNA dependent procedure. The dual bead complexes are subjected to DNAases (enzymes that specifically cut DNA). This treatment separates the reporter beads from the capture beads by cutting the DNA that holds them together. Thus, the non-target mediated dual beads will not be affected. The reporter beads that are released after the DNAse treatment are indicative of the amount of target DNA present in the sample. In this experiment, the DNAseI effect in a dual bead assay was evaluated.

A. Dual Bead Assay

The dual bead assay was carried out as described previously in Example 1, Part A. Briefly, the assay is comprised of 3 μm magnetic capture beads (Spherotech, Libertyville, Ill.) coated with covalently attached transport probes; 2.1 μm fluorescent reporter beads (Molecular Probes, Eugene, Oreg.) coated with a covalently attached reporter probes, and target DNA molecules of interest. In this example, the target DNA is a synthetic 80 oligonucleotides long. The transport probes and reporter probes are 40 nucleotides in length and are complementary to the target DNA but not to each other.

The specific methodology employed to prepare the assay involved treating 1×10⁷ capture beads and 2×10⁷ reporter beads in 100 μg/ml salmon sperm DNA for 1 hour at room temperature. This pre-treatment will reduce the non-specific binding between the capture and reporter beads in the absence of target DNA. The capture beads were concentrated magnetically with the supernatant being removed. A 100 μl volume of the hybridization buffer (0.2M NaCl, 1 mM EDTA, 1 mM MgCl₂, 50 mM Tris-HCl, pH 7.5 and 5× Denhart's mix, 10 μg/ml denatured salmon sperm DNA) were added and the beads were resuspended. Various concentration of target DNA ranging from 1, 10, 100 and 1000 femtomoles were added to the capture bead suspensions. The beads suspension was incubated while mixing at 37 degrees Centigrade for 2 hours. The beads were magnetically concentrated and the supernatant containing unbound target DNA was removed. One hundred microliters of wash buffer (145 mM NaCl, 50 mM Tris, pH 7.5, 0.1% SDS, 0.05% Tween, 0.25% NFDM (Non Fat Dried Milk), 10 mM EDTA) was added and the beads were resuspended. The beads were magnetically concentrated and the supernatant was again removed. The wash procedure was repeated twice.

A 2×10⁷ amount of reporter beads in 100 μl hybridization buffer (0.2M NaCl, 1 mM EDTA, 10 mM MgCl₂, 50 mM Tris-HCl, pH 7.5 and 5× Denhart's mix, 10 g/ml denatured salmon sperm DNA) were then added to washed capture beads. The beads were resuspended and incubated while mixing at 37 degrees Centigrade for an additional 2 hours. After incubation, the capture beads were concentrated magnetically, and the supernatant containing unbound reporter beads were removed. One hundred microliters of wash buffer (145 mM NaCl, 50 mM Tris, pH 7.5, 0.1% SDS, 0.05% Tween, 0.25% NFDM (Non Fat Dried Milk), 10 mM EDTA) was added and the beads were resuspended. The beads were magnetically concentrated and the supernatant was again removed. The wash procedure was repeated twice.

B. DNAseI Assays

DNAseI was selected for this purpose because it is not sequence specific. Following washing, the dual bead complexes were resuspended in 87.5 μL water. Ten (10) units of DNAseI (2.5 μL) and 10 μL of DNAseI reaction buffer (40 mM Tris-HCl, 10 mM MgSO₄, 1 mM CaCl₂) were added to the re-suspended beads. The digestion reaction was carried out for 1 hour at 37° C. After digestion, the capture beads were concentrated magnetically and the supernatant containing reporter beads was removed. The magnetic capture beads were washed 2 times with 100 μl water. The washed water was combined with the supernatant. The number of reporter beads was quantified by the fluorimeter Fluoromax-2 at excitation 500 nm, emission 530 nm and slit sizes 2.0. Alternatively, the number of fluorescent reporter beads can be quantified by the bio-disc reader as described previously.

### Example 4

In this example, the dual bead complexes are separated by physical or chemical treatments. The dual bead assay was carried out as described above in Example 3. Following washing, the bead products were washed 5 times with the wash buffer (145 mM NaCl, 50 mM Tris, pH 7.5, 0.1% SDS, 0.05% Tween, 0.25% NFDM (Non Fat Dried Milk), 10 mM EDTA) and divided into four sets.
1. Control: No treatment, the beads were washed twice with 200 μL wash buffer.
2. Acid Wash: The beads were washed twice with 200 μL wash buffer containing 0.1M acetic acid (pH 4).
3. Basic Wash: The beads were washed twice with 200 μL wash buffer containing 0.1 M sodium bicarbonate (pH 9).
4. Urea: The beads were washed twice with 200 μL wash buffer containing 7M urea.

After the physical or chemical treatment, the capture beads were concentrated magnetically, and the supernatant containing released reporter beads was saved. The beads were washed 3 times with wash buffer. The wash was also saved. The magnetic capture beads were re-suspended in 400 μL wash buffer. The amount of reporter beads in the supernatants and in the solution of capture beads were quantified by the fluorimeter Fluoromax-2 at Ex=500 nm, Slit=2.0; Em=530 nm, Slit=2.0. Alternatively, the number of fluorescent reporter beads can be quantified by the bio-disc reader as described above.

As evident by this example, high pH washes can dissociate the reporter beads from the capture beads at low target concentrations. As shown by the experimental results in FIG. 41, the basic wash completely dissociated the reporter beads from capture beads at low target concentrations.

The results of this experiment also established that a 7M urea treatment efficiently dissociates reporter beads from capture beads without significantly compromising the sensitivity. As illustrated by the experimental results present in the bar graphs of FIGS. 42A and 42B, urea treatment efficiently dissociates reporter beads from capture beads.

### Example 5

In the examples discussed above, the target DNA is single stranded. When clinical samples are used, the DNA is double stranded and therefore the hybridization buffer requires a denaturing reagent such as guanidine isothiocyanate. The concentration of the denaturing reagent used in the assay is a major contributor in the specificity and sensitivity of the dual bead assay. In this example, the dual bead assay to detect HSV was carried out in the presence of 1.5M guanidine isothiocyanate.

A. Preparation of Capture Beads

The dual bead assay is comprised of 3 μm magnetic capture beads (Spherotech, Libertyville, Ill.) coated with covalently attached 5′ HSV transport probes and 2.1 μm fluorescent reporter beads from Molecular Probes (Eugene, Oreg.) conjugated to the 3′ HSV reporter probes and target DNA molecules of interest. In this example, the target was a double-strand PCR product containing the HSV gene sequence, amplified for 30 cycles and Qiagen column purified. The transport probes and reporter probes are 40 nucleotides in length and are complementary to the target DNA but not to each other.

The specific methodology employed to prepare the assay involved treating 1×10⁷ capture beads and 2×10⁷ reporter beads in 100 μg/ml salmon sperm DNA for 1 hour at room temperature. This pre-treatment will reduce the non-specific binding between the capture and reporter beads in the absence of target DNA. The capture beads were concentrated magnetically with the supernatant being removed. The capture beads were resuspended in 600 μL of hybridization buffer (1.5 GuSCN, 8 mM EDTA, 40 mM Tris, pH7.5) containing 5× Denhart's mix, and 10 μg/ml salmon sperm DNA.

B. Preparation of Target DNA

The target was a double-strand PCR product, amplified for 30 cycles and Qiagen column purified. The target was diluted to appropriate concentrations, and heated at 95° C. for 5 minutes to denature the double strand then quickly chilled on ice.

C. Hybridization with the Target DNA

A total of 12.5 μL of the chilled target was added to the 100 μL of the pretreated capture beads. Various concentrations of target DNA ranging from 0, 10^{-16, 10}^{-15, 10}^{-14, 10}⁻¹³, and 10⁻¹² moles were added to the capture bead suspensions. The beads suspension was incubated while mixing at 37 degrees Centigrade for 2 hours. The beads were magnetically concentrated and the supernatant containing unbound target DNA was removed. One hundred microliters of wash buffer (145 mM NaCl, 50 mM Tris, pH 7.5, 0.1% SDS, 0.05% Tween, 0.25% NFDM (Non Fat Dried Milk), 10 mM EDTA) was added and the beads were resuspended. The beads were magnetically concentrated and the supernatant was again removed. The wash procedure was repeated twice.

D. Dual Bead Assay

A 2×10⁷ amount of reporter beads in 100 μl hybridization buffer (1.5 GuSCN, 8 mM EDTA, 40 mM Tris, pH7.4) containing 5× Denhart's mix and 10 μg/ml denatured salmon sperm DNA) were then added to washed capture beads. The beads were resuspended and incubated while mixing at 37 degrees Centigrade for an additional 3 hours. After incubation, the capture beads were concentrated magnetically, and the supernatant containing unbound reporter beads were removed. One hundred microliters of wash buffer (145 mM NaCl, 50 mM Tris, pH 7.5, 0.1% SDS, 0.05% Tween, 0.25% NFDM (Non Fat Dried Milk), 10 mM EDTA) was added and the beads were resuspended. The beads were magnetically concentrated and the supernatant was again removed. The wash procedure was repeated twice.

E. Quantification of Target DNA

The dual bead complexes were resuspended in 250 μl PBS and the amount of target was quantified by fluorescence measurement of the reporter beads using the fluorimeter Fluoromax-2 at Ex=500 nm, Slit=2.0; Em=530 nm, Slit=2.0. Alternatively, the number of fluorescent reporter beads can be quantified by use of the optical bio-disc reader as described above.

F. HSV Glycoprotein B Gene Structure

The structure of the HSV glycoprotein B gene utilized in this experiment is provided below in Table 1. As indicated, this structure is 292 base pairs long. The probes are highlighted by use of bold text.

TABLE 1

SEQ ID NO:1

5′CCAACGCCGCGACCCGCACGAGCCGGGGCTGGCACACCACCG
ACCTCAGTACAACCCCTCGCGGGTGAGGCGTTCCACCGGTACGGGA
CGACGGTAAACTGCATCGTCGAGGAGGTGGACGCGCGCTCGGTGTA
CCCGTACGACGAGTTTGTGCTGGCGACTGGCGACTTTGTGTACATGT
CCCCGTTTTACGGCTACCGGGAGGGTCGCACACCGAACACACCAGC
TACGCCGCCGACCGCTTCAAGCAGGTTGACGGCTTCTACGCGCG
CGACCTCACCACCAAGG 3′

### Example 6

The following example illustrates a dual bead assay carried out on a magnetically writable and erasable analysis disc such as the magneto-optical bio-disc 110 discussed in conjunction with FIG. 37.

In this example, the dual bead assay is carried out to detect the gene sequence DYS which is present in male but not female. The assay is comprised of 3 μm magnetic capture beads (Spherotech, Libertyville, Ill.) coated with covalently attached transport probes; 2.1 μm fluorescent reporter beads (Molecular Probes, Eugene, Oreg.) coated with a covalently attached sequence specific for the DYS gene, and target DNA molecules containing DYS sequences. The target DNA is a synthetic 80 oligonucleotides long. The transport probes and reporter probes are 40 nucleotides in length and are complementary to the DYS sequence but not to each other.

The specific methodology employed to prepare the assay involved treating 1×10⁷ capture beads and 2×10⁷ reporter beads in 100 μg/ml salmon sperm DNA for 1 hour at room temperature. This pre-treatment will reduce the non-specific binding between the capture and reporter beads in the absence of target DNA.

After pretreatment with salmon sperm DNA, the capture beads are loaded inside the MO bio-disc via the injection port. The MO bio-disc contains magnetic regions created by the magneto optical drive. The capture beads thus are held within specific magnetic regions on the MO bio-disc.

The sample containing target DNA and reporter beads in 200 μl hybridization buffer (0.2M NaCl, 1 mM EDTA, 10 mM MgCl₂, 50 mM Tris-HCl, pH 7.5 and 5× Denhart's mix, 10 μg/ml denatured salmon sperm DNA) is then added to the MO bio-disc via the injection port. The injection port is then sealed. The magnetic field is released. The disc is rotated at very low speed (less than 800 rpm) in the drive to facilitate hybridization of target DNA and reporter beads to the capture beads. The temperature of the drive is kept constant at 33 degrees Centigrade. After 2 hours of hybridization, the magnetic field is created by the magneto optical drive. At this stage, only magnetic capture beads, unbound or as part of a dual bead complex, remain on the MO bio-disc. Unbound target and reporter beads are directed to a waste chamber by any of the mechanisms described above. Two hundred microliters of wash buffer (145 mM NaCl, 50 mM Tris, pH 7.5, 0.1% SDS, 0.05% Tween, 0.25% NFDM (Non Fat Dried Milk), 10 mM EDTA) is then added. The magnetic field is released and the disc is rotated at low speed (less than 800 rpm) for 5 minutes to remove any non-specific binding between the capture beads and reporter beads. The magnetic field is then reapplied. The wash buffer is directed to the waste chamber by any of the mechanisms described above. The wash procedure is repeated twice.

At this stage, only magnetic capture beads, unbound or as part of a dual bead complex, remain. The magnetic field is released and the dual bead complexes are directed to a detection chamber. The amount of target DNA captured is then enumerated by quantifying the number of capture magnetic beads and the number of reporter beads since each type of bead has a distinct signature as illustrated above in FIGS. 28A, 28B, 29A, and 29B.

### Example 7

In this example, a dual bead assay using the multiplexing techniques described above in connection with FIGS. 32 and 37 is carried out on a magnetically writable and erasable analysis disc such as the MO bio-disc 110 discussed with reference to FIG. 37.

The dual bead assay is carried out to detect 2 or more DNA targets simultaneously. The assay is comprised of 3 μm magnetic capture beads (Spherotech, Libertyville, Ill.). One population of the magnetic capture beads is coated with transport probes 1 which are complementary to the DNA target 1. Another population of the magnetic capture beads is coated with transport probes 2 which are complementary to the DNA target 2. Alternatively, 2 or more different types of magnetic capture beads may be used. There are two or more distinct types of reporter beads in the assay. The reporter beads may differ by chemical composition (for example silica and polystyrene) and/or by size. One type of reporter beads is coated with reporter probes 1, which are complementary to the DNA target 1. The other reporter beads are coated with reporter probes 2, which are complementary to the DNA target 2. Again, the transport probes and reporter probes are complementary to the respective targets but not to each other.

The specific methodology employed to prepare the dual bead assay multiplexing involved treating 1×10⁷ capture beads and 2×10⁷ reporter beads in 100 μg/ml salmon sperm DNA for 1 hour at room temperature. This pre-treatment will reduce the non-specific binding between the capture and reporter beads in the absence of target DNA.

After pretreatment with salmon sperm DNA, the capture beads are loaded in the MO bio-disc. The magnetic field is applied to create distinct magnetic zones for specific capture beads. The capture beads can be held on the MO bio-disc at a density of 1 capture bead per 10 μm². The surface area usable for bead deposition on the MO bio-disc is approximately 3×10⁹ μm². The capacity of the MO bio-disc for 3 μm beads at the given density is about 3×10⁸ beads.

The sample containing the targets DNA of interest is mixed with different types of reporter beads in 200 μl hybridization buffer (0.2M NaCl, 1 mM EDTA, 10 mM MgCl₂, 50 mM Tris-HCl, pH 7.5 and 5× Denhart's mix, 10 μg/ml denatured salmon sperm DNA) and added to the MO bio-disc via the injection port. The injection port is then sealed. The magnetic field is released. The disc is rotated at very low speed (less than 800 rpm) in the drive to facilitate hybridization of targets DNA and reporter beads to the different types of capture beads. The temperature of the drive is kept constant at 33 degrees Centigrade. After 2 to 3 hours of hybridization, the magnetic field is regenerated by the magneto optical drive. At this stage, only magnetic capture beads, unbound or as part of dual bead complexes, remain on the MO bio-disc. Unbound targets and reporter beads are directed to a waste chamber by any of the mechanisms described above. Two hundred microliters of wash buffer (145 mM NaCl, 50 mM Tris, pH 7.5, 0.1% SDS, 0.05% Tween, 0.25% NFDM (Non Fat Dried Milk), 10 mM EDTA) is then added. The magnetic field is released and the disc is rotated at low speed (less than 800 rpm) for 5 minutes to remove any non-specific binding between the capture beads and reporter beads. The magnetic field is then reapplied. The wash buffer is directed to the waste chamber by any of the mechanisms described above. The wash procedure is repeated twice.

At this stage, the magnetic field is released and the dual bead complexes are directed to a detection chamber. The amount of different types of target DNA can be enumerated by quantifying the number of corresponding capture magnetic beads and reporter beads since each type of bead has a distinct signature as shown above in FIGS. 28A, 28B, 29A, and 29B.

### CONCLUDING SUMMARY

While this invention has been described in detail with reference to certain preferred embodiments and technical examples, it should be appreciated that the present invention is not limited to those precise embodiments or examples. Rather, in view of the present disclosure, which describes the current best mode for practicing the invention, many modifications and variations would present themselves to those of skill in the art without departing from the scope and spirit of this invention. For example, any of the off-disc preparation procedures may be readily performed on disc by use of suitable fluidic circuits employing the methods described herein. Also, any of the fluidic circuits discussed in connection with the reflective and transmissive discs may be readily adapted to the MO bio-disc. In addition, the scope of the present invention is not solely limited to the formation of only dual bead complexes. The methods and apparatus hereof may be readily applied to the creation of multi-bead assays. For example, a single capture bead may bind multiple reporter beads. Similarly, a single reporter bead may bind multiple capture beads. Furthermore, linked chains of multi-bead or dual bead complexes may be formed by target mediated binding between capture and reporter beads. The linked chains may further agglutinate to thereby increase detectability of a target agent of interest. The scope of the invention is, therefore, indicated by the following claims rather than by the foregoing description. All changes, modifications, and variations coming within the meaning and range of equivalency of the claims are to be considered within their scope.

What is claimed is:

1. An optical bio-disc, comprising:
a substrate having encoded information associated therewith, said encoded information being readable by a disc drive assembly to control rotation of the disc;
a target zone associated with said substrate, said target zone disposed at a predetermined location relative to said substrate;
an active layer associated with said target zone;
a plurality of capture agents attached to said active layer so that when said substrate is rotated, said capture agents remain attached to said active layer to thereby maintain a number of said capture agents within said target zone; and
a dual bead complex attached to said capture agents, wherein said dual bead complex has been pre-washed in a buffer containing a dissociation agent.

2. The optical bio-disc according to claim 1, wherein said capture agent is selected from the group consisting of a single stranded oligonucleotide sequence, a double stranded oligonucleotide sequence, an antibody, an antigen, biotin, and streptavidin.

3. The optical bio-disc according to claim 1, wherein said active layer is disposed between said substrate and a cap.

4. The optical bio-disc according to claim 1, wherein said active layer is selected from the group consisting of nitrocellulose, polystyrene, polycarbonate, gold, activated glass, modified glass, and modified polystyrene.

5. The optical bio-disc according to claim 1, wherein said dissociation agent is an enzyme.

6. The optical bio-disc according to claim 5, wherein said enzyme is a DNase.

7. The optical bio-disc according to claim 1, wherein said dissociation is a chaotropic agent.

8. The optical bio-disc according to claim 7, wherein said chaotropic agent is urea.

9. The optical bio-disc according to claim 7, wherein said chaotropic agent is guanidine isothiocyanate.

10. An optical bio-disc assay system, comprising:
a substrate having encoded information associated therewith, said encoded information being readable by a disc drive assembly to control rotation of the disc;
a target zone associated with said substrate, said target zone disposed at a predetermined location relative to said substrate;
an active layer associated with said target zone;
a plurality of capture agents attached to said active layer so that when said substrate is rotated, said capture agents remain attached to said active layer to thereby maintain a number of said capture agents within said target zone; and
a dual bead complex which interacts with said capture agents, wherein said dual bead complex has been pre-washed in a buffer containing a dissociation agent.

11. The optical bio-disc bio-disc according to claim 10, wherein said capture agent is selected from the group consisting of a single stranded oligonucleotide sequence, a double stranded oligonucleotide sequence, an antibody, an antigen, biotin, or and streptavidin.

12. The optical bio-disc according to claim 10, wherein said active layer is disposed between said substrate and a cap.

13. The optical bio-disc according to claim 10, wherein said active layer is selected from the group consisting of nitrocellulose, polystyrene, polycarbonate, gold, activated glass, modified glass, and modified polystyrene.

14. The optical bio-disc according to claim 10, wherein said dissociation agent is an enzyme.

15. The optical bio-disc according to claim 14, wherein said enzyme is a DNase.

16. The optical bio-disc according to claim 10, wherein said dissociation is a chaotropic agent.

17. The optical bio-disc according to claim 16, wherein said chaotropic agent is urea.

18. The optical bio-disc according to claim 16, wherein said chaotropic agent is guanidine isothiocyanate.


SEQUENCE LISTING (1 sequence)

SEQ ID NO: 1 - DNA, 288 bp, HSV
ccaacgccgcgacccgcacgagccggggctggcacaccaccgacctcagtacaacccctcgcgggtgaggcgttccaccggtacgggacgacggtaaactgcatcgtcgaggaggtggacgcgcgctcggtgtacccgtacgacgagtttgtgctggcgactggcgactttgtgtacatgtccccgttttacggctaccgggagggtcgcacaccgaacacaccagctacgccgccgaccgcttcaagcaggttgacggcttctacgcgcgcgacctcaccaccaagg